SEQUENCE LISTING (328 sequences)

SEQ ID NO: 1 - RNA, 22 nt, Rattus sp.
  misc_feature (1)..(22): Rat siRNA
aacgauuuccuagccugggcau

SEQ ID NO: 2 - RNA, 22 nt, Rattus sp.
  misc_feature (1)..(22): Rat Passenger Strand Sequence
  misc_feature (21)..(22): n is a, c, g, or u
gcccaggcuaggaaaucguunn

SEQ ID NO: 3 - RNA, 22 nt, Rattus sp.
  misc_feature (1)..(22): Rat siRNA
uuaaugggacuuucugaaccac

SEQ ID NO: 4 - RNA, 22 nt, Rattus sp.
  misc_feature (1)..(22): Rat Passenger Strand Sequence
  misc_feature (21)..(22): n is a, c, g, or u
gguucagaaagucccauuagnn

SEQ ID NO: 5 - RNA, 22 nt, Rattus sp.
  misc_feature (1)..(22): Rat siRNA
ugcuuauguuuggccagaccag

SEQ ID NO: 6 - RNA, 22 nt, Rattus sp.
  misc_feature (1)..(22): Rat Passenger Strand Sequence
  misc_feature (21)..(22): n is a, c, g, or u
ggucuggccaaacauaaguann

SEQ ID NO: 7 - RNA, 22 nt, Rattus sp.
  misc_feature (1)..(22): Rat siRNA
agaucuugcagacacaaggcaa

SEQ ID NO: 8 - RNA, 22 nt, Rattus sp.
  misc_feature (1)..(22): Rat Passenger Strand Sequence
  misc_feature (21)..(22): n is a, c, g, or u
gccuugugucugcaagauuunn

SEQ ID NO: 9 - RNA, 22 nt, Rattus sp.
  misc_feature (1)..(22): Rat siRNA
agcucuauugcagugagggcaa

SEQ ID NO: 10 - RNA, 22 nt, Rattus sp.
  misc_feature (1)..(22): Rat Passenger Strand Sequence
  misc_feature (21)..(22): n is a, c, g, or u
gcccucacugcaauagaguunn

SEQ ID NO: 11 - RNA, 22 nt, Rattus sp.
  misc_feature (1)..(22): Rat siRNA
gagugggucugcagaugugccc

SEQ ID NO: 12 - RNA, 22 nt, Rattus sp.
  misc_feature (1)..(22): Rat Passenger Strand Sequence
  misc_feature (21)..(22): n is a, c, g, or u
gcacaucugcagacccauuunn

SEQ ID NO: 13 - RNA, 22 nt, Rattus sp.
  misc_feature (1)..(22): Rat siRNA
ggucuuauugcauaaauugcac

SEQ ID NO: 14 - RNA, 22 nt, Rattus sp.
  misc_feature (1)..(22): Rat Passenger Strand Sequence
  misc_feature (21)..(22): n is a, c, g, or u
gcaauuuaugcaauaagauunn

SEQ ID NO: 15 - RNA, 22 nt, Rattus sp.
  misc_feature (1)..(22): Rat siRNA
uguccauacaguuauagggcug

SEQ ID NO: 16 - RNA, 22 nt, Rattus sp.
  misc_feature (1)..(22): Rat Passenger Strand Sequence
  misc_feature (21)..(22): n is a, c, g, or u
gcccuauaacuguauggauann

SEQ ID NO: 17 - RNA, 22 nt, Rattus sp.
  misc_feature (1)..(22): Rat siRNA
uagacuccucauguuuaugcag

SEQ ID NO: 18 - RNA, 22 nt, Rattus sp.
  misc_feature (1)..(22): Rat Passenger Strand Sequence
  misc_feature (21)..(22): n is a, c, g, or u
gcauaaacaugaggaguuugnn

SEQ ID NO: 19 - RNA, 22 nt, Rattus sp.
  misc_feature (1)..(22): Rat siRNA
uagugugccacacagcagccag

SEQ ID NO: 20 - RNA, 22 nt, Rattus sp.
  misc_feature (1)..(22): Rat Passenger Strand Sequence
  misc_feature (21)..(22): n is a, c, g, or u
ggcugcuguguggcacauugnn

SEQ ID NO: 21 - RNA, 22 nt, Rattus sp.
  misc_feature (1)..(22): Rat siRNA
guuauagggcuguacgcuccug

SEQ ID NO: 22 - RNA, 22 nt, Rattus sp.
  misc_feature (1)..(22): Rat Passenger Strand Sequence
  misc_feature (21)..(22): n is a, c, g, or u
ggagcguacagcccuauaaunn

SEQ ID NO: 23 - RNA, 22 nt, Rattus sp.
  misc_feature (1)..(22): Rat siRNA
gugggucugcagaugugcccuc

SEQ ID NO: 24 - RNA, 22 nt, Rattus sp.
  misc_feature (1)..(22): Rat Passenger Strand Sequence
  misc_feature (21)..(22): n is a, c, g, or u
gggcacaucugcagaccuaunn

SEQ ID NO: 25 - RNA, 22 nt, Rattus sp.
  misc_feature (1)..(22): Rat siRNA
aguagaauaggucuuauugcau

SEQ ID NO: 26 - RNA, 22 nt, Rattus sp.
  misc_feature (1)..(22): Rat Passenger Strand Sequence
  misc_feature (21)..(22): n is a, c, g, or u
gcaauaagaccuauucuauunn

SEQ ID NO: 27 - RNA, 22 nt, Rattus sp.
  misc_feature (1)..(22): Rat siRNA
uucuaauguguccuugaagcag

SEQ ID NO: 28 - RNA, 22 nt, Rattus sp.
  misc_feature (1)..(22): Rat Passenger Strand Sequence
  misc_feature (21)..(22): n is a, c, g, or u
gcuucaaggacacauuagaann

SEQ ID NO: 29 - RNA, 22 nt, Rattus sp.
  misc_feature (1)..(22): Rat siRNA
agccuugccacagaucuugcag

SEQ ID NO: 30 - RNA, 22 nt, Rattus sp.
  misc_feature (1)..(22): Rat Passenger Strand Sequence
  misc_feature (21)..(22): n is a, c, g, or u
gcaagaucuguggcaagguunn

SEQ ID NO: 31 - RNA, 22 nt, Rattus sp.
  misc_feature (1)..(22): Rat siRNA
gggggucugaaagcuugggcug

SEQ ID NO: 32 - RNA, 22 nt, Rattus sp.
  misc_feature (1)..(22): Rat Passenger Strand Sequence
  misc_feature (21)..(22): n is a, c, g, or u
gcccaagcuuucagaccuuunn

SEQ ID NO: 33 - RNA, 22 nt, Rattus sp.
  misc_feature (1)..(22): Rat siRNA
uuacaucagagugggucugcag

SEQ ID NO: 34 - RNA, 22 nt, Rattus sp.
  misc_feature (1)..(22): Rat Passenger Strand Sequence
  misc_feature (21)..(22): n is a, c, g, or u
gcagacccacucugauguaann

SEQ ID NO: 35 - RNA, 22 nt, Rattus sp.
  misc_feature (1)..(22): Rat siRNA
aguaagaggagaaaggccgcug

SEQ ID NO: 36 - RNA, 22 nt, Rattus sp.
  misc_feature (1)..(22): Rat Passenger Strand Sequence
  misc_feature (21)..(22): n is a, c, g, or u
gcggccuuucuccucuuauunn

SEQ ID NO: 37 - RNA, 22 nt, Rattus sp.
  misc_feature (1)..(22): Rat siRNA
gggcaucacagugcagcugcuu

SEQ ID NO: 38 - RNA, 22 nt, Rattus sp.
  misc_feature (1)..(22): Rat Passenger Strand Sequence
  misc_feature (21)..(22): n is a, c, g, or u
gcagcugcacugugauguuunn

SEQ ID NO: 39 - RNA, 22 nt, Rattus sp.
  misc_feature (1)..(22): Rat siRNA
agcugaacgauuuccuagccug

SEQ ID NO: 40 - RNA, 22 nt, Rattus sp.
  misc_feature (1)..(22): Rat Passenger Strand Sequence
  misc_feature (21)..(22): n is a, c, g, or u
ggcuaggaaaucguucaguunn

SEQ ID NO: 41 - RNA, 22 nt, Rattus sp.
  misc_feature (1)..(22): Rat siRNA
ugugcaucuucagggcacccag

SEQ ID NO: 42 - RNA, 22 nt, Rattus sp.
  misc_feature (1)..(22): Rat Passenger Strand Sequence
  misc_feature (21)..(22): n is a, c, g, or u
gggugcccugaagaugcauann

SEQ ID NO: 43 - RNA, 22 nt, Rattus sp.
  misc_feature (1)..(22): Rat siRNA
agugggucugcagaugugcccu

SEQ ID NO: 44 - RNA, 22 nt, Rattus sp.
  misc_feature (1)..(22): Rat Passenger Strand Sequence
  misc_feature (21)..(22): n is a, c, g, or u
ggcacaucugcagacccauunn

SEQ ID NO: 45 - RNA, 22 nt, Rattus sp.
  misc_feature (1)..(22): Rat siRNA
uuugcagcugaacgauuuccua

SEQ ID NO: 46 - RNA, 22 nt, Rattus sp.
  misc_feature (1)..(22): Rat Passenger Strand Sequence
  misc_feature (21)..(22): n is a, c, g, or u
ggaaaucguucagcugcaaann

SEQ ID NO: 47 - RNA, 22 nt, Rattus sp.
  misc_feature (1)..(22): Rat siRNA
uugcacugaaacuucucagcuu

SEQ ID NO: 48 - RNA, 22 nt, Rattus sp.
  misc_feature (1)..(22): Rat Passenger Strand Sequence
  misc_feature (21)..(22): n is a, c, g, or u
gcugagaaguuucaguguaann

SEQ ID NO: 49 - RNA, 22 nt, Rattus sp.
  misc_feature (1)..(22): Rat siRNA
gggucuggagaaagccuugcca

SEQ ID NO: 50 - RNA, 22 nt, Rattus sp.
  misc_feature (1)..(22): Rat Passenger Strand Sequence
  misc_feature (21)..(22): n is a, c, g, or u
gcaaggcuuucuccagauuunn

SEQ ID NO: 51 - RNA, 22 nt, Homo sapiens
  misc_feature (1)..(22): Human siRNA
gauugcgucacucagugugcua

SEQ ID NO: 52 - RNA, 22 nt, Homo sapiens
  misc_feature (1)..(22): Human Passenger Strand Sequence
  misc_feature (21)..(22): n is a, c, g, or u
gcacacugagugacgcaauunn

SEQ ID NO: 53 - RNA, 22 nt, Homo sapiens
  misc_feature (1)..(22): Human siRNA
uucgaguaaacauugauugcgu

SEQ ID NO: 54 - RNA, 22 nt, Homo sapiens
  misc_feature (1)..(22): Human Passenger Strand Sequence
  misc_feature (21)..(22): n is a, c, g, or u
gcaaucaauguuuacucgaann

SEQ ID NO: 55 - RNA, 22 nt, Homo sapiens
  misc_feature (1)..(22): Human siRNA
gacacggcggucccuacagcau

SEQ ID NO: 56 - RNA, 22 nt, Homo sapiens
  misc_feature (1)..(22): Human Passenger Strand Sequence
  misc_feature (21)..(22): n is a, c, g, or u
gcuguagggaccgccguguunn

SEQ ID NO: 57 - RNA, 22 nt, Homo sapiens
  misc_feature (1)..(22): Human siRNA
ugggcguggaauggagcagcgg

SEQ ID NO: 58 - RNA, 22 nt, Homo sapiens
  misc_feature (1)..(22): Human Passenger Strand Sequence
  misc_feature (21)..(22): n is a, c, g, or u
gcugcuccauuccacgcuuann

SEQ ID NO: 59 - RNA, 22 nt, Homo sapiens
  misc_feature (1)..(22): Human siRNA
uuugaccugucugcaaaugcuc

SEQ ID NO: 60 - RNA, 22 nt, Homo sapiens
  misc_feature (1)..(22): Human Passenger Strand Sequence
  misc_feature (21)..(22): n is a, c, g, or u
gcauuugcagacaggucaaann

SEQ ID NO: 61 - RNA, 22 nt, Homo sapiens
  misc_feature (1)..(22): Human siRNA
gggcaucgcagugcagcugcuu

SEQ ID NO: 62 - RNA, 22 nt, Homo sapiens
  misc_feature (1)..(22): Human Passenger Strand Sequence
  misc_feature (21)..(22): n is a, c, g, or u
gcagcugcacugcgauguuunn

SEQ ID NO: 63 - RNA, 22 nt, Homo sapiens
  misc_feature (1)..(22): Human siRNA
agguuguuuauagacauagcua

SEQ ID NO: 64 - RNA, 22 nt, Homo sapiens
  misc_feature (1)..(22): Human Passenger Strand Sequence
  misc_feature (21)..(22): n is a, c, g, or u
gcuaugucuauaaacaauuunn

SEQ ID NO: 65 - RNA, 22 nt, Homo sapiens
  misc_feature (1)..(22): Human siRNA
aaugggucugcagaugagcccu

SEQ ID NO: 66 - RNA, 22 nt, Homo sapiens
  misc_feature (1)..(22): Human Passenger Strand Sequence
  misc_feature (21)..(22): n is a, c, g, or u
ggcucaucugcagacccauunn

SEQ ID NO: 67 - RNA, 22 nt, Homo sapiens
  misc_feature (1)..(22): Human siRNA
agcuaaucuuucccuccucccc

SEQ ID NO: 68 - RNA, 22 nt, Homo sapiens
  misc_feature (1)..(22): Human Passenger Strand Sequence
  misc_feature (21)..(22): n is a, c, g, or u
ggaggagggaaagauuaguunn

SEQ ID NO: 69 - RNA, 22 nt, Homo sapiens
  misc_feature (1)..(22): Human siRNA
ggucuuauugcauaaauugcac

SEQ ID NO: 70 - RNA, 22 nt, Homo sapiens
  misc_feature (1)..(22): Human Passenger Strand Sequence
  misc_feature (21)..(22): n is a, c, g, or u
gcaauuuaugcaauaagauunn

SEQ ID NO: 71 - RNA, 22 nt, Homo sapiens
  misc_feature (1)..(22): Human siRNA
ugcuuauguuuggccagcccag

SEQ ID NO: 72 - RNA, 22 nt, Homo sapiens
  misc_feature (1)..(22): Human Passenger Strand Sequence
  misc_feature (21)..(22): n is a, c, g, or u
gggcuggccaaacauaaguann

SEQ ID NO: 73 - RNA, 22 nt, Homo sapiens
  misc_feature (1)..(22): Human siRNA
uuaguucaacaauggcaaccag

SEQ ID NO: 74 - RNA, 22 nt, Homo sapiens
  misc_feature (1)..(22): Human Passenger Strand Sequence
  misc_feature (21)..(22): n is a, c, g, or u
gguugccauuguugaacuaann

SEQ ID NO: 75 - RNA, 22 nt, Homo sapiens
  misc_feature (1)..(22): Human siRNA
uugguugugguaugacaggcau

SEQ ID NO: 76 - RNA, 22 nt, Homo sapiens
  misc_feature (1)..(22): Human Passenger Strand Sequence
  misc_feature (21)..(22): n is a, c, g, or u
gccugucauaccacaacuagnn

SEQ ID NO: 77 - RNA, 22 nt, Homo sapiens
  misc_feature (1)..(22): Human siRNA
uuaauggggcuuucugagccac

SEQ ID NO: 78 - RNA, 22 nt, Homo sapiens
  misc_feature (1)..(22): Human Passenger Strand Sequence
  misc_feature (21)..(22): n is a, c, g, or u
ggcucagaaagccccauuagnn

SEQ ID NO: 79 - RNA, 22 nt, Homo sapiens
  misc_feature (1)..(22): Human siRNA
aguaagacaauggagcaugcgc

SEQ ID NO: 80 - RNA, 22 nt, Homo sapiens
  misc_feature (1)..(22): Human Passenger Strand Sequence
  misc_feature (21)..(22): n is a, c, g, or u
gcaugcuccauugucuuauunn

SEQ ID NO: 81 - RNA, 22 nt, Homo sapiens
  misc_feature (1)..(22): Human siRNA
aagggguauuguauuauugcau

SEQ ID NO: 82 - RNA, 22 nt, Homo sapiens
  misc_feature (1)..(22): Human Passenger Strand Sequence
  misc_feature (21)..(22): n is a, c, g, or u
gcaauaauacaauacccuuunn

SEQ ID NO: 83 - RNA, 22 nt, Homo sapiens
  misc_feature (1)..(22): Human siRNA
aaggagcgcggcaucuugccag

SEQ ID NO: 84 - RNA, 22 nt, Homo sapiens
  misc_feature (1)..(22): Human Passenger Strand Sequence
  misc_feature (21)..(22): n is a, c, g, or u
ggcaagaugccgcgcucuuunn

SEQ ID NO: 85 - RNA, 22 nt, Homo sapiens
  misc_feature (1)..(22): Human siRNA
uuugguacuaaucaugaagcaa

SEQ ID NO: 86 - RNA, 22 nt, Homo sapiens
  misc_feature (1)..(22): Human Passenger Strand Sequence
  misc_feature (21)..(22): n is a, c, g, or u
gcuucaugauuaguaccaaann

SEQ ID NO: 87 - RNA, 22 nt, Homo sapiens
  misc_feature (1)..(22): Human siRNA
aggccauuggguagcugggcgu

SEQ ID NO: 88 - RNA, 22 nt, Homo sapiens
  misc_feature (1)..(22): Human Passenger Strand Sequence
  misc_feature (21)..(22): n is a, c, g, or u
gcccagcuacccaaugguuunn

SEQ ID NO: 89 - RNA, 22 nt, Homo sapiens
  misc_feature (1)..(22): Human siRNA
augggucugcagaugagcccuc

SEQ ID NO: 90 - RNA, 22 nt, Homo sapiens
  misc_feature (1)..(22): Human Passenger Strand Sequence
  misc_feature (21)..(22): n is a, c, g, or u
gggcucaucugcagaccuaunn

SEQ ID NO: 91 - RNA, 22 nt, Homo sapiens
  misc_feature (1)..(22): Human siRNA
aguugaauaggucuuauugcau

SEQ ID NO: 92 - RNA, 22 nt, Homo sapiens
  misc_feature (1)..(22): Human Passenger Strand Sequence
  misc_feature (21)..(22): n is a, c, g, or u
gcaauaagaccuauucaauunn

SEQ ID NO: 93 - RNA, 22 nt, Homo sapiens
  misc_feature (1)..(22): Human siRNA
aguccacacagugauggggcug

SEQ ID NO: 94 - RNA, 22 nt, Homo sapiens
  misc_feature (1)..(22): Human Passenger Strand Sequence
  misc_feature (21)..(22): n is a, c, g, or u
gccccaucacuguguggauunn

SEQ ID NO: 95 - RNA, 22 nt, Homo sapiens
  misc_feature (1)..(22): Human siRNA
uagauuccucauguuugugcag

SEQ ID NO: 96 - RNA, 22 nt, Homo sapiens
  misc_feature (1)..(22): Human Passenger Strand Sequence
  misc_feature (21)..(22): n is a, c, g, or u
gcacaaacaugaggaauuugnn

SEQ ID NO: 97 - RNA, 22 nt, Homo sapiens
  misc_feature (1)..(22): Human siRNA
uagugugcuacacagcagccag

SEQ ID NO: 98 - RNA, 22 nt, Homo sapiens
  misc_feature (1)..(22): Human Passenger Strand Sequence
  misc_feature (21)..(22): n is a, c, g, or u
ggcugcuguguagcacauugnn

SEQ ID NO: 99 - RNA, 22 nt, Homo sapiens
  misc_feature (1)..(22): Human siRNA
gugauggggcuguaugcuccug

SEQ ID NO: 100 - RNA, 22 nt, Homo sapiens
  misc_feature (1)..(22): Human Passenger Strand Sequence
  misc_feature (21)..(22): n is a, c, g, or u
ggagcauacagccccauuaunn

SEQ ID NO: 101 - RNA, 22 nt, Mus musculus
  misc_feature (1)..(22): Mouse siRNA
gguugcgccacucagugugcca

SEQ ID NO: 102 - RNA, 22 nt, Mus musculus
  misc_feature (1)..(22): Mouse Passenger Strand Sequence
  misc_feature (21)..(22): n is a, c, g, or u
gcacacugaguggcgcaauunn

SEQ ID NO: 103 - RNA, 22 nt, Mus musculus
  misc_feature (1)..(22): Mouse siRNA
guugcgccacucagugugccac

SEQ ID NO: 104 - RNA, 22 nt, Mus musculus
  misc_feature (1)..(22): Mouse Passenger Strand Sequence
  misc_feature (21)..(22): n is a, c, g, or u
ggcacacugaguggcgcaaunn

SEQ ID NO: 105 - RNA, 22 nt, Mus musculus
  misc_feature (1)..(22): Mouse siRNA
aacgguccaggcggcgguccua

SEQ ID NO: 106 - RNA, 22 nt, Mus musculus
  misc_feature (1)..(22): Mouse Passenger Strand Sequence
  misc_feature (21)..(22): n is a, c, g, or u
ggaccgccgccuggaccguunn

SEQ ID NO: 107 - RNA, 22 nt, Mus musculus
  misc_feature (1)..(22): Mouse siRNA
aacgauuuccuagacugggcau

SEQ ID NO: 108 - RNA, 22 nt, Mus musculus
  misc_feature (1)..(22): Mouse Passenger Strand Sequence
  misc_feature (21)..(22): n is a, c, g, or u
gcccagucuaggaaaucguunn

SEQ ID NO: 109 - RNA, 22 nt, Mus musculus
  misc_feature (1)..(22): Mouse siRNA
uauaggcguggcuauuaaccgu

SEQ ID NO: 110 - RNA, 22 nt, Mus musculus
  misc_feature (1)..(22): Mouse Passenger Strand Sequence
  misc_feature (21)..(22): n is a, c, g, or u
gguuaauagccacgccuauann

SEQ ID NO: 111 - RNA, 22 nt, Mus musculus
  misc_feature (1)..(22): Mouse siRNA
ggaguacuuuauaaggcaccug

SEQ ID NO: 112 - RNA, 22 nt, Mus musculus
  misc_feature (1)..(22): Mouse Passenger Strand Sequence
  misc_feature (21)..(22): n is a, c, g, or u
ggugccuuauaaaguacuuunn

SEQ ID NO: 113 - RNA, 22 nt, Mus musculus
  misc_feature (1)..(22): Mouse siRNA
agcccuauugcagugagggcaa

SEQ ID NO: 114 - RNA, 22 nt, Mus musculus
  misc_feature (1)..(22): Mouse Passenger Strand Sequence
  misc_feature (21)..(22): n is a, c, g, or u
gcccucacugcaauaggguunn

SEQ ID NO: 115 - RNA, 22 nt, Mus musculus
  misc_feature (1)..(22): Mouse siRNA
aaggcgggggacuuacacgccc

SEQ ID NO: 116 - RNA, 22 nt, Mus musculus
  misc_feature (1)..(22): Mouse Passenger Strand Sequence
  misc_feature (21)..(22): n is a, c, g, or u
gcguguaagucccccgcuuunn

SEQ ID NO: 117 - RNA, 22 nt, Mus musculus
  misc_feature (1)..(22): Mouse siRNA
ugguugaauauaggcguggcua

SEQ ID NO: 118 - RNA, 22 nt, Mus musculus
  misc_feature (1)..(22): Mouse Passenger Strand Sequence
  misc_feature (21)..(22): n is a, c, g, or u
gccacgccuauauucaauuann

SEQ ID NO: 119 - RNA, 22 nt, Mus musculus
  misc_feature (1)..(22): Mouse siRNA
ggcauuguauuacugcauccau

SEQ ID NO: 120 - RNA, 22 nt, Mus musculus
  misc_feature (1)..(22): Mouse Passenger Strand Sequence
  misc_feature (21)..(22): n is a, c, g, or u
ggaugcaguaauacaauguunn

SEQ ID NO: 121 - RNA, 22 nt, Mus musculus
  misc_feature (1)..(22): Mouse siRNA
ggucuuauugcauaaauugcac

SEQ ID NO: 122 - RNA, 22 nt, Mus musculus
  misc_feature (1)..(22): Mouse Passenger Strand Sequence
  misc_feature (21)..(22): n is a, c, g, or u
gcaauuuaugcaauaagauunn

SEQ ID NO: 123 - RNA, 22 nt, Mus musculus
  misc_feature (1)..(22): Mouse siRNA
uaauagggcuguaugcucccga

SEQ ID NO: 124 - RNA, 22 nt, Mus musculus
  misc_feature (1)..(22): Mouse Passenger Strand Sequence
  misc_feature (21)..(22): n is a, c, g, or u
gggagcauacagcccuauuann

SEQ ID NO: 125 - RNA, 22 nt, Mus musculus
  misc_feature (1)..(22): Mouse siRNA
uguccauacaguaauagggcug

SEQ ID NO: 126 - RNA, 22 nt, Mus musculus
  misc_feature (1)..(22): Mouse Passenger Strand Sequence
  misc_feature (21)..(22): n is a, c, g, or u
gcccuauuacuguauggauann

SEQ ID NO: 127 - RNA, 22 nt, Mus musculus
  misc_feature (1)..(22): Mouse siRNA
ugcuuguguuuggccagcccag

SEQ ID NO: 128 - RNA, 22 nt, Mus musculus
  misc_feature (1)..(22): Mouse Passenger Strand Sequence
  misc_feature (21)..(22): n is a, c, g, or u
gggcuggccaaacacaaguann

SEQ ID NO: 129 - RNA, 22 nt, Mus musculus
  misc_feature (1)..(22): Mouse siRNA
uuaaugggacuuucugaaccac

SEQ ID NO: 130 - RNA, 22 nt, Mus musculus
  misc_feature (1)..(22): Mouse Passenger Strand Sequence
  misc_feature (21)..(22): n is a, c, g, or u
gguucagaaagucccauuagnn

SEQ ID NO: 131 - RNA, 22 nt, Mus musculus
  misc_feature (1)..(22): Mouse siRNA
ugugcaucuucagggcacccag

SEQ ID NO: 132 - RNA, 22 nt, Mus musculus
  misc_feature (1)..(22): Mouse Passenger Strand Sequence
  misc_feature (21)..(22): n is a, c, g, or u
gggugcccugaagaugcauann

SEQ ID NO: 133 - RNA, 22 nt, Mus musculus
  misc_feature (1)..(22): Mouse siRNA
uaguugggcuucuuggaggcgu

SEQ ID NO: 134 - RNA, 22 nt, Mus musculus
  misc_feature (1)..(22): Mouse Passenger Strand Sequence
  misc_feature (21)..(22): n is a, c, g, or u
gccuccaagaagcccaauuann

SEQ ID NO: 135 - RNA, 22 nt, Mus musculus
  misc_feature (1)..(22): Mouse siRNA
aaggagcgcggcaugguggcug

SEQ ID NO: 136 - RNA, 22 nt, Mus musculus
  misc_feature (1)..(22): Mouse Passenger Strand Sequence
  misc_feature (21)..(22): n is a, c, g, or u
gccaccaugccgcgcucuuunn

SEQ ID NO: 137 - RNA, 22 nt, Mus musculus
  misc_feature (1)..(22): Mouse siRNA
ggagaguggaguggagcugccg

SEQ ID NO: 138 - RNA, 22 nt, Mus musculus
  misc_feature (1)..(22): Mouse Passenger Strand Sequence
  misc_feature (21)..(22): n is a, c, g, or u
gcagcuccacuccacucuuunn

SEQ ID NO: 139 - RNA, 22 nt, Mus musculus
  misc_feature (1)..(22): Mouse siRNA
guaauagggcuguaugcucccg

SEQ ID NO: 140 - RNA, 22 nt, Mus musculus
  misc_feature (1)..(22): Mouse Passenger Strand Sequence
  misc_feature (21)..(22): n is a, c, g, or u
ggagcauacagcccuauuaunn

SEQ ID NO: 141 - RNA, 22 nt, Mus musculus
  misc_feature (1)..(22): Mouse siRNA
agaucuugcagacacaaggcaa

SEQ ID NO: 142 - RNA, 22 nt, Mus musculus
  misc_feature (1)..(22): Mouse Passenger Strand Sequence
  misc_feature (21)..(22): n is a, c, g, or u
gccuugugucugcaagauuunn

SEQ ID NO: 143 - RNA, 22 nt, Mus musculus
  misc_feature (1)..(22): Mouse siRNA
uagacuccucauguuuaugcag

SEQ ID NO: 144 - RNA, 22 nt, Mus musculus
  misc_feature (1)..(22): Mouse Passenger Strand Sequence
  misc_feature (21)..(22): n is a, c, g, or u
gcauaaacaugaggaguuugnn

SEQ ID NO: 145 - RNA, 22 nt, Mus musculus
  misc_feature (1)..(22): Mouse siRNA
gggcaucacagugcagcugcuu

SEQ ID NO: 146 - RNA, 22 nt, Mus musculus
  misc_feature (1)..(22): Mouse Passenger Strand Sequence
  misc_feature (21)..(22): n is a, c, g, or u
gcagcugcacugugauguuunn

SEQ ID NO: 147 - RNA, 22 nt, Mus musculus
  misc_feature (1)..(22): Mouse siRNA
gugggucugcagaugugcccuc

SEQ ID NO: 148 - RNA, 22 nt, Mus musculus
  misc_feature (1)..(22): Mouse Passenger Strand Sequence
  misc_feature (21)..(22): n is a, c, g, or u
gggcacaucugcagaccuaunn

SEQ ID NO: 149 - RNA, 22 nt, Mus musculus
  misc_feature (1)..(22): Mouse siRNA
uuugaguaaacacugguugcgc

SEQ ID NO: 150 - RNA, 22 nt, Mus musculus
  misc_feature (1)..(22): Mouse Passenger Strand Sequence
  misc_feature (21)..(22): n is a, c, g, or u
gcaaccaguguuuacucaaann

SEQ ID NO: 151 - RNA, 22 nt, Rattus sp.
  misc_feature (1)..(22): Rat siRNA
uaaucggugggucugggagcag

SEQ ID NO: 152 - RNA, 22 nt, Rattus sp.
  misc_feature (1)..(22): Rat Passenger Strand Sequence
  misc_feature (21)..(22): n is a, c, g, or u
gcucccagacccaccgauuann

SEQ ID NO: 153 - RNA, 22 nt, Rattus sp.
  misc_feature (1)..(22): Rat siRNA
uguucgcugaagccaaguccgg

SEQ ID NO: 154 - RNA, 22 nt, Rattus sp.
  misc_feature (1)..(22): Rat Passenger Strand Sequence
  misc_feature (21)..(22): n is a, c, g, or u
ggacuuggcuucagcgaaugnn

SEQ ID NO: 155 - RNA, 22 nt, Rattus sp.
  misc_feature (1)..(22): Rat siRNA
uuguacggagaggcuguccccc

SEQ ID NO: 156 - RNA, 22 nt, Rattus sp.
  misc_feature (1)..(22): Rat Passenger Strand Sequence
  misc_feature (21)..(22): n is a, c, g, or u
gggacagccucuccguauagnn

SEQ ID NO: 157 - RNA, 22 nt, Rattus sp.
  misc_feature (1)..(22): Rat siRNA
guugccgcuccagaacgugccc

SEQ ID NO: 158 - RNA, 22 nt, Rattus sp.
  misc_feature (1)..(22): Rat Passenger Strand Sequence
  misc_feature (21)..(22): n is a, c, g, or u
gcacguucuggagcggcaaunn

SEQ ID NO: 159 - RNA, 22 nt, Rattus sp.
  misc_feature (1)..(22): Rat siRNA
uguacggagaggcuguccccca

SEQ ID NO: 160 - RNA, 22 nt, Rattus sp.
  misc_feature (1)..(22): Rat Passenger Strand Sequence
  misc_feature (21)..(22): n is a, c, g, or u
ggggacagccucuccguauann

SEQ ID NO: 161 - RNA, 22 nt, Rattus sp.
  misc_feature (1)..(22): Rat siRNA
uucgaaucaacaccaaacgcug

SEQ ID NO: 162 - RNA, 22 nt, Rattus sp.
  misc_feature (1)..(22): Rat Passenger Strand Sequence
  misc_feature (21)..(22): n is a, c, g, or u
gcguuugguguugauucgagnn

SEQ ID NO: 163 - RNA, 22 nt, Rattus sp.
  misc_feature (1)..(22): Rat siRNA
uuucguccgccugugccgccac

SEQ ID NO: 164 - RNA, 22 nt, Rattus sp.
  misc_feature (1)..(22): Rat Passenger Strand Sequence
  misc_feature (21)..(22): n is a, c, g, or u
ggcggcacaggcggacgaaann

SEQ ID NO: 165 - RNA, 22 nt, Rattus sp.
  misc_feature (1)..(22): Rat siRNA
uuaacgcucgaaucaacaccaa

SEQ ID NO: 166 - RNA, 22 nt, Rattus sp.
  misc_feature (1)..(22): Rat Passenger Strand Sequence
  misc_feature (21)..(22): n is a, c, g, or u
gguguugauucgagcguuaann

SEQ ID NO: 167 - RNA, 22 nt, Rattus sp.
  misc_feature (1)..(22): Rat siRNA
ggugucgagguugccagggcua

SEQ ID NO: 168 - RNA, 22 nt, Rattus sp.
  misc_feature (1)..(22): Rat Passenger Strand Sequence
  misc_feature (21)..(22): n is a, c, g, or u
gcccuggcaaccucgacauunn

SEQ ID NO: 169 - RNA, 22 nt, Rattus sp.
  misc_feature (1)..(22): Rat siRNA
uugccgcuccagaacgugcccg

SEQ ID NO: 170 - RNA, 22 nt, Rattus sp.
  misc_feature (1)..(22): Rat Passenger Strand Sequence
  misc_feature (21)..(22): n is a, c, g, or u
ggcacguucuggagcgguaann

SEQ ID NO: 171 - RNA, 22 nt, Rattus sp.
  misc_feature (1)..(22): Rat siRNA
uacgcuccaaggccugcagcug

SEQ ID NO: 172 - RNA, 22 nt, Rattus sp.
  misc_feature (1)..(22): Rat Passenger Strand Sequence
  misc_feature (21)..(22): n is a, c, g, or u
gcugcaggccuuggagcgugnn

SEQ ID NO: 173 - RNA, 22 nt, Rattus sp.
  misc_feature (1)..(22): Rat siRNA
guacggagaggcugucccccag

SEQ ID NO: 174 - RNA, 22 nt, Rattus sp.
  misc_feature (1)..(22): Rat Passenger Strand Sequence
  misc_feature (21)..(22): n is a, c, g, or u
gggggacagccucuccguaunn

SEQ ID NO: 175 - RNA, 22 nt, Rattus sp.
  misc_feature (1)..(22): Rat siRNA
uuuucguccgccugugccgcca

SEQ ID NO: 176 - RNA, 22 nt, Rattus sp.
  misc_feature (1)..(22): Rat Passenger Strand Sequence
  misc_feature (21)..(22): n is a, c, g, or u
gcggcacaggcggacgaaagnn

SEQ ID NO: 177 - RNA, 22 nt, Rattus sp.
  misc_feature (1)..(22): Rat siRNA
uucgguucguucgcugaagcca

SEQ ID NO: 178 - RNA, 22 nt, Rattus sp.
  misc_feature (1)..(22): Rat Passenger Strand Sequence
  misc_feature (21)..(22): n is a, c, g, or u
gcuucagcgaacgaaccgaann

SEQ ID NO: 179 - RNA, 22 nt, Rattus sp.
  misc_feature (1)..(22): Rat siRNA
uucgguucugaaaccacaccug

SEQ ID NO: 180 - RNA, 22 nt, Rattus sp.
  misc_feature (1)..(22): Rat Passenger Strand Sequence
  misc_feature (21)..(22): n is a, c, g, or u
ggugugguuucagaaccgaann

SEQ ID NO: 181 - RNA, 22 nt, Rattus sp.
  misc_feature (1)..(22): Rat siRNA
auggcgcuguacggagaggcug

SEQ ID NO: 182 - RNA, 22 nt, Rattus sp.
  misc_feature (1)..(22): Rat Passenger Strand Sequence
  misc_feature (21)..(22): n is a, c, g, or u
gccucuccguacagcgcuaunn

SEQ ID NO: 183 - RNA, 22 nt, Rattus sp.
  misc_feature (1)..(22): Rat siRNA
ugccgcuccagaacgugcccgu

SEQ ID NO: 184 - RNA, 22 nt, Rattus sp.
  misc_feature (1)..(22): Rat Passenger Strand Sequence
  misc_feature (21)..(22): n is a, c, g, or u
gggcacguucuggagcgguann

SEQ ID NO: 185 - RNA, 22 nt, Rattus sp.
  misc_feature (1)..(22): Rat siRNA
agacacgcuccaaggccugcag

SEQ ID NO: 186 - RNA, 22 nt, Rattus sp.
  misc_feature (1)..(22): Rat Passenger Strand Sequence
  misc_feature (21)..(22): n is a, c, g, or u
gcaggccuuggagcguguuunn

SEQ ID NO: 187 - RNA, 22 nt, Rattus sp.
  misc_feature (1)..(22): Rat siRNA
ugcgcagggcuauuguuggcac

SEQ ID NO: 188 - RNA, 22 nt, Rattus sp.
  misc_feature (1)..(22): Rat Passenger Strand Sequence
  misc_feature (21)..(22): n is a, c, g, or u
gccaacaauagcccugcguann

SEQ ID NO: 189 - RNA, 22 nt, Rattus sp.
  misc_feature (1)..(22): Rat siRNA
uauuggccagcauggcucgcuc

SEQ ID NO: 190 - RNA, 22 nt, Rattus sp.
  misc_feature (1)..(22): Rat Passenger Strand Sequence
  misc_feature (21)..(22): n is a, c, g, or u
gcgagccaugcuggccaauann

SEQ ID NO: 191 - RNA, 22 nt, Rattus sp.
  misc_feature (1)..(22): Rat siRNA
agcauccggguaauggguccuc

SEQ ID NO: 192 - RNA, 22 nt, Rattus sp.
  misc_feature (1)..(22): Rat Passenger Strand Sequence
  misc_feature (21)..(22): n is a, c, g, or u
ggacccauuacccggauguunn

SEQ ID NO: 193 - RNA, 22 nt, Rattus sp.
  misc_feature (1)..(22): Rat siRNA
uguuggccauguugauacccug

SEQ ID NO: 194 - RNA, 22 nt, Rattus sp.
  misc_feature (1)..(22): Rat Passenger Strand Sequence
  misc_feature (21)..(22): n is a, c, g, or u
ggguaucaacauggccaauann

SEQ ID NO: 195 - RNA, 22 nt, Rattus sp.
  misc_feature (1)..(22): Rat siRNA
uauggucuucccuccaauccca

SEQ ID NO: 196 - RNA, 22 nt, Rattus sp.
  misc_feature (1)..(22): Rat Passenger Strand Sequence
  misc_feature (21)..(22): n is a, c, g, or u
ggauuggagggaagaccaugnn

SEQ ID NO: 197 - RNA, 22 nt, Rattus sp.
  misc_feature (1)..(22): Rat siRNA
agguuggcaaugcuguuggcca

SEQ ID NO: 198 - RNA, 22 nt, Rattus sp.
  misc_feature (1)..(22): Rat Passenger Strand Sequence
  misc_feature (21)..(22): n is a, c, g, or u
gccaacagcauugccaauuunn

SEQ ID NO: 199 - RNA, 22 nt, Rattus sp.
  misc_feature (1)..(22): Rat siRNA
uuguuggccauguugauacccu

SEQ ID NO: 200 - RNA, 22 nt, Rattus sp.
  misc_feature (1)..(22): Rat Passenger Strand Sequence
  misc_feature (21)..(22): n is a, c, g, or u
gguaucaacauggccaauagnn

SEQ ID NO: 201 - RNA, 22 nt, Homo sapiens
  misc_feature (1)..(22): Human siRNA
uguacggagacgcuguccccca

SEQ ID NO: 202 - RNA, 22 nt, Homo sapiens
  misc_feature (1)..(22): Human Passenger Strand Sequence
  misc_feature (21)..(22): n is a, c, g, or u
ggggacagcgucuccguauann

SEQ ID NO: 203 - RNA, 22 nt, Homo sapiens
  misc_feature (1)..(22): Human siRNA
uuguacggagacgcuguccccc

SEQ ID NO: 204 - RNA, 22 nt, Homo sapiens
  misc_feature (1)..(22): Human Passenger Strand Sequence
  misc_feature (21)..(22): n is a, c, g, or u
gggacagcgucuccguauagnn

SEQ ID NO: 205 - RNA, 22 nt, Homo sapiens
  misc_feature (1)..(22): Human siRNA
uaaucggugggucucggagcag

SEQ ID NO: 206 - RNA, 22 nt, Homo sapiens
  misc_feature (1)..(22): Human Passenger Strand Sequence
  misc_feature (21)..(22): n is a, c, g, or u
gcuccgagacccaccgauuann

SEQ ID NO: 207 - RNA, 22 nt, Homo sapiens
  misc_feature (1)..(22): Human siRNA
guacggagacgcugucccccag

SEQ ID NO: 208 - RNA, 22 nt, Homo sapiens
  misc_feature (1)..(22): Human Passenger Strand Sequence
  misc_feature (21)..(22): n is a, c, g, or u
gggggacagcgucuccguaunn

SEQ ID NO: 209 - RNA, 22 nt, Homo sapiens
  misc_feature (1)..(22): Human siRNA
uucgguucugaaaccacaccug

SEQ ID NO: 210 - RNA, 22 nt, Homo sapiens
  misc_feature (1)..(22): Human Passenger Strand Sequence
  misc_feature (21)..(22): n is a, c, g, or u
ggugugguuucagaaccgaann

SEQ ID NO: 211 - RNA, 22 nt, Homo sapiens
  misc_feature (1)..(22): Human siRNA
guguccgcucaaagacacgcuc

SEQ ID NO: 212 - RNA, 22 nt, Homo sapiens
  misc_feature (1)..(22): Human Passenger Strand Sequence
  misc_feature (21)..(22): n is a, c, g, or u
gcgugucuuugagcggauaunn

SEQ ID NO: 213 - RNA, 22 nt, Homo sapiens
  misc_feature (1)..(22): Human siRNA
uacgcuccaaagccugcagcug

SEQ ID NO: 214 - RNA, 22 nt, Homo sapiens
  misc_feature (1)..(22): Human Passenger Strand Sequence
  misc_feature (21)..(22): n is a, c, g, or u
gcugcaggcuuuggagcgugnn

SEQ ID NO: 215 - RNA, 22 nt, Homo sapiens
  misc_feature (1)..(22): Human siRNA
uauccgccugugccgccaccau

SEQ ID NO: 216 - RNA, 22 nt, Homo sapiens
  misc_feature (1)..(22): Human Passenger Strand Sequence
  misc_feature (21)..(22): n is a, c, g, or u
gguggcggcacaggcggaugnn

SEQ ID NO: 217 - RNA, 22 nt, Homo sapiens
  misc_feature (1)..(22): Human siRNA
ggcacgaaagagaugugggcau

SEQ ID NO: 218 - RNA, 22 nt, Homo sapiens
  misc_feature (1)..(22): Human Passenger Strand Sequence
  misc_feature (21)..(22): n is a, c, g, or u
gcccacaucucuuucguguunn

SEQ ID NO: 219 - RNA, 22 nt, Homo sapiens
  misc_feature (1)..(22): Human siRNA
ugcgugcuguucagcaagccua

SEQ ID NO: 220 - RNA, 22 nt, Homo sapiens
  misc_feature (1)..(22): Human Passenger Strand Sequence
  misc_feature (21)..(22): n is a, c, g, or u
ggcuugcugaacagcacguann

SEQ ID NO: 221 - RNA, 22 nt, Homo sapiens
  misc_feature (1)..(22): Human siRNA
uaggacgagguacgaugggcug

SEQ ID NO: 222 - RNA, 22 nt, Homo sapiens
  misc_feature (1)..(22): Human Passenger Strand Sequence
  misc_feature (21)..(22): n is a, c, g, or u
gcccaucguaccucgucuugnn

SEQ ID NO: 223 - RNA, 22 nt, Homo sapiens
  misc_feature (1)..(22): Human siRNA
augcgugcuguucagcaagccu

SEQ ID NO: 224 - RNA, 22 nt, Homo sapiens
  misc_feature (1)..(22): Human Passenger Strand Sequence
  misc_feature (21)..(22): n is a, c, g, or u
gcuugcugaacagcacguaunn

SEQ ID NO: 225 - RNA, 22 nt, Homo sapiens
  misc_feature (1)..(22): Human siRNA
agacacgcuccaaagccugcag

SEQ ID NO: 226 - RNA, 22 nt, Homo sapiens
  misc_feature (1)..(22): Human Passenger Strand Sequence
  misc_feature (21)..(22): n is a, c, g, or u
gcaggcuuuggagcguguuunn

SEQ ID NO: 227 - RNA, 22 nt, Homo sapiens
  misc_feature (1)..(22): Human siRNA
gacgagguacgaugggcugcuc

SEQ ID NO: 228 - RNA, 22 nt, Homo sapiens
  misc_feature (1)..(22): Human Passenger Strand Sequence
  misc_feature (21)..(22): n is a, c, g, or u
gcagcccaucguaccucguunn

SEQ ID NO: 229 - RNA, 22 nt, Homo sapiens
  misc_feature (1)..(22): Human siRNA
ggcgggcaaggucuucucgcac

SEQ ID NO: 230 - RNA, 22 nt, Homo sapiens
  misc_feature (1)..(22): Human Passenger Strand Sequence
  misc_feature (21)..(22): n is a, c, g, or u
gcgagaagaccuugcccguunn

SEQ ID NO: 231 - RNA, 22 nt, Homo sapiens
  misc_feature (1)..(22): Human siRNA
gagguacgaugggcugcuccac

SEQ ID NO: 232 - RNA, 22 nt, Homo sapiens
  misc_feature (1)..(22): Human Passenger Strand Sequence
  misc_feature (21)..(22): n is a, c, g, or u
ggagcagcccaucguacuuunn

SEQ ID NO: 233 - RNA, 22 nt, Homo sapiens
  misc_feature (1)..(22): Human siRNA
uauacccuaaaugucauucccu

SEQ ID NO: 234 - RNA, 22 nt, Homo sapiens
  misc_feature (1)..(22): Human Passenger Strand Sequence
  misc_feature (21)..(22): n is a, c, g, or u
ggaaugacauuuaggguauann

SEQ ID NO: 235 - RNA, 22 nt, Homo sapiens
  misc_feature (1)..(22): Human siRNA
agaauccguuaugaagccccuc

SEQ ID NO: 236 - RNA, 22 nt, Homo sapiens
  misc_feature (1)..(22): Human Passenger Strand Sequence
  misc_feature (21)..(22): n is a, c, g, or u
ggggcuucauaacggauuuunn

SEQ ID NO: 237 - RNA, 22 nt, Homo sapiens
  misc_feature (1)..(22): Human siRNA
uggaguacauuaagcaaagcau

SEQ ID NO: 238 - RNA, 22 nt, Homo sapiens
  misc_feature (1)..(22): Human Passenger Strand Sequence
  misc_feature (21)..(22): n is a, c, g, or u
gcuuugcuuaauguacuuuann

SEQ ID NO: 239 - RNA, 22 nt, Homo sapiens
  misc_feature (1)..(22): Human siRNA
uguauuguuugggaaauugcaa

SEQ ID NO: 240 - RNA, 22 nt, Homo sapiens
  misc_feature (1)..(22): Human Passenger Strand Sequence
  misc_feature (21)..(22): n is a, c, g, or u
gcaauuucccaaacaauauann

SEQ ID NO: 241 - RNA, 22 nt, Homo sapiens
  misc_feature (1)..(22): Human siRNA
agguuguccuauuccuucgcug

SEQ ID NO: 242 - RNA, 22 nt, Homo sapiens
  misc_feature (1)..(22): Human Passenger Strand Sequence
  misc_feature (21)..(22): n is a, c, g, or u
gcgaaggaauaggacaauuunn

SEQ ID NO: 243 - RNA, 22 nt, Homo sapiens
  misc_feature (1)..(22): Human siRNA
auagagccucacaacaccccug

SEQ ID NO: 244 - RNA, 22 nt, Homo sapiens
  misc_feature (1)..(22): Human Passenger Strand Sequence
  misc_feature (21)..(22): n is a, c, g, or u
gggguguugugaggcucuaunn

SEQ ID NO: 245 - RNA, 22 nt, Homo sapiens
  misc_feature (1)..(22): Human siRNA
auacccuaaaugucauucccuu

SEQ ID NO: 246 - RNA, 22 nt, Homo sapiens
  misc_feature (1)..(22): Human Passenger Strand Sequence
  misc_feature (21)..(22): n is a, c, g, or u
gggaaugacauuuaggguaunn

SEQ ID NO: 247 - RNA, 22 nt, Homo sapiens
  misc_feature (1)..(22): Human siRNA
uguaguuggucaaguccaccug

SEQ ID NO: 248 - RNA, 22 nt, Homo sapiens
  misc_feature (1)..(22): Human Passenger Strand Sequence
  misc_feature (21)..(22): n is a, c, g, or u
gguggacuugaccaacuauann

SEQ ID NO: 249 - RNA, 22 nt, Homo sapiens
  misc_feature (1)..(22): Human siRNA
gguuaugucuugacauuugcag

SEQ ID NO: 250 - RNA, 22 nt, Homo sapiens
  misc_feature (1)..(22): Human Passenger Strand Sequence
  misc_feature (21)..(22): n is a, c, g, or u
gcaaaugucaagacauaauunn

SEQ ID NO: 251 - RNA, 22 nt, Mus musculus
  misc_feature (1)..(22): Mouse siRNA
agcgcguucccccucucugcac

SEQ ID NO: 252 - RNA, 22 nt, Mus musculus
  misc_feature (1)..(22): Mouse Passenger Strand Sequence
  misc_feature (21)..(22): n is a, c, g, or u
gcagagagggggaacgcguunn

SEQ ID NO: 253 - RNA, 22 nt, Mus musculus
  misc_feature (1)..(22): Mouse siRNA
uaaucgguuggucugggagcag

SEQ ID NO: 254 - RNA, 22 nt, Mus musculus
  misc_feature (1)..(22): Mouse Passenger Strand Sequence
  misc_feature (21)..(22): n is a, c, g, or u
gcucccagaccaaccgauuann

SEQ ID NO: 255 - RNA, 22 nt, Mus musculus
  misc_feature (1)..(22): Mouse siRNA
uuguacggagaggcuguccccc

SEQ ID NO: 256 - RNA, 22 nt, Mus musculus
  misc_feature (1)..(22): Mouse Passenger Strand Sequence
  misc_feature (21)..(22): n is a, c, g, or u
gggacagccucuccguauagnn

SEQ ID NO: 257 - RNA, 22 nt, Mus musculus
  misc_feature (1)..(22): Mouse siRNA
guugccgcuccagaacgugccc

SEQ ID NO: 258 - RNA, 22 nt, Mus musculus
  misc_feature (1)..(22): Mouse Passenger Strand Sequence
  misc_feature (21)..(22): n is a, c, g, or u
gcacguucuggagcggcaaunn

SEQ ID NO: 259 - RNA, 22 nt, Mus musculus
  misc_feature (1)..(22): Mouse siRNA
uguacggagaggcuguccccca

SEQ ID NO: 260 - RNA, 22 nt, Mus musculus
  misc_feature (1)..(22): Mouse Passenger Strand Sequence
  misc_feature (21)..(22): n is a, c, g, or u
ggggacagccucuccguauann

SEQ ID NO: 261 - RNA, 22 nt, Mus musculus
  misc_feature (1)..(22): Mouse siRNA
uauccggguaauguguccgcuc

SEQ ID NO: 262 - RNA, 22 nt, Mus musculus
  misc_feature (1)..(22): Mouse Passenger Strand Sequence
  misc_feature (21)..(22): n is a, c, g, or u
gcggacacauuacccggaugnn

SEQ ID NO: 263 - RNA, 22 nt, Mus musculus
  misc_feature (1)..(22): Mouse siRNA
guguccgcucaaagacacgcuc

SEQ ID NO: 264 - RNA, 22 nt, Mus musculus
  misc_feature (1)..(22): Mouse Passenger Strand Sequence
  misc_feature (21)..(22): n is a, c, g, or u
gcgugucuuugagcggauaunn

SEQ ID NO: 265 - RNA, 22 nt, Mus musculus
  misc_feature (1)..(22): Mouse siRNA
uucgaaucaacaccaaacgcug

SEQ ID NO: 266 - RNA, 22 nt, Mus musculus
  misc_feature (1)..(22): Mouse Passenger Strand Sequence
  misc_feature (21)..(22): n is a, c, g, or u
gcguuugguguugauucgagnn

SEQ ID NO: 267 - RNA, 22 nt, Mus musculus
  misc_feature (1)..(22): Mouse siRNA
uuaacggugacaucauaaccua

SEQ ID NO: 268 - RNA, 22 nt, Mus musculus
  misc_feature (1)..(22): Mouse Passenger Strand Sequence
  misc_feature (21)..(22): n is a, c, g, or u
gguuaugaugucaccguuaann

SEQ ID NO: 269 - RNA, 22 nt, Mus musculus
  misc_feature (1)..(22): Mouse siRNA
uugccgcuccagaacgugcccg

SEQ ID NO: 270 - RNA, 22 nt, Mus musculus
  misc_feature (1)..(22): Mouse Passenger Strand Sequence
  misc_feature (21)..(22): n is a, c, g, or u
ggcacguucuggagcgguaann

SEQ ID NO: 271 - RNA, 22 nt, Mus musculus
  misc_feature (1)..(22): Mouse siRNA
uucgguuacuucgcuaaagcca

SEQ ID NO: 272 - RNA, 22 nt, Mus musculus
  misc_feature (1)..(22): Mouse Passenger Strand Sequence
  misc_feature (21)..(22): n is a, c, g, or u
gcuuuagcgaaguaaccgagnn

SEQ ID NO: 273 - RNA, 22 nt, Mus musculus
  misc_feature (1)..(22): Mouse siRNA
guacggagaggcugucccccag

SEQ ID NO: 274 - RNA, 22 nt, Mus musculus
  misc_feature (1)..(22): Mouse Passenger Strand Sequence
  misc_feature (21)..(22): n is a, c, g, or u
gggggacagccucuccguaunn

SEQ ID NO: 275 - RNA, 22 nt, Mus musculus
  misc_feature (1)..(22): Mouse siRNA
uuuucgucugcuugugccgcca

SEQ ID NO: 276 - RNA, 22 nt, Mus musculus
  misc_feature (1)..(22): Mouse Passenger Strand Sequence
  misc_feature (21)..(22): n is a, c, g, or u
gcggcacaagcagacgaaagnn

SEQ ID NO: 277 - RNA, 22 nt, Mus musculus
  misc_feature (1)..(22): Mouse siRNA
uuucgucugcuugugccgccac

SEQ ID NO: 278 - RNA, 22 nt, Mus musculus
  misc_feature (1)..(22): Mouse Passenger Strand Sequence
  misc_feature (21)..(22): n is a, c, g, or u
ggcggcacaagcagacgaaann

SEQ ID NO: 279 - RNA, 22 nt, Mus musculus
  misc_feature (1)..(22): Mouse siRNA
gacgagguacgauggguugcuc

SEQ ID NO: 280 - RNA, 22 nt, Mus musculus
  misc_feature (1)..(22): Mouse Passenger Strand Sequence
  misc_feature (21)..(22): n is a, c, g, or u
gcaacccaucguaccucguunn

SEQ ID NO: 281 - RNA, 22 nt, Mus musculus
  misc_feature (1)..(22): Mouse siRNA
uucgguucugaaaccacaccug

SEQ ID NO: 282 - RNA, 22 nt, Mus musculus
  misc_feature (1)..(22): Mouse Passenger Strand Sequence
  misc_feature (21)..(22): n is a, c, g, or u
ggugugguuucagaaccgaann

SEQ ID NO: 283 - RNA, 22 nt, Mus musculus
  misc_feature (1)..(22): Mouse siRNA
auggcgcuguacggagaggcug

SEQ ID NO: 284 - RNA, 22 nt, Mus musculus
  misc_feature (1)..(22): Mouse Passenger Strand Sequence
  misc_feature (21)..(22): n is a, c, g, or u
gccucuccguacagcgcuaunn

SEQ ID NO: 285 - RNA, 22 nt, Mus musculus
  misc_feature (1)..(22): Mouse siRNA
uucgggacaaguaacuuucccu

SEQ ID NO: 286 - RNA, 22 nt, Mus musculus
  misc_feature (1)..(22): Mouse Passenger Strand Sequence
  misc_feature (21)..(22): n is a, c, g, or u
ggaaaguuacuugucccgaann

SEQ ID NO: 287 - RNA, 22 nt, Mus musculus
  misc_feature (1)..(22): Mouse siRNA
aagcgccaacagaagaaggcuu

SEQ ID NO: 288 - RNA, 22 nt, Mus musculus
  misc_feature (1)..(22): Mouse Passenger Strand Sequence
  misc_feature (21)..(22): n is a, c, g, or u
gccuucuucuguuggcguuunn

SEQ ID NO: 289 - RNA, 22 nt, Mus musculus
  misc_feature (1)..(22): Mouse siRNA
ugccgcuccagaacgugcccgu

SEQ ID NO: 290 - RNA, 22 nt, Mus musculus
  misc_feature (1)..(22): Mouse Passenger Strand Sequence
  misc_feature (21)..(22): n is a, c, g, or u
gggcacguucuggagcgguann

SEQ ID NO: 291 - RNA, 22 nt, Mus musculus
  misc_feature (1)..(22): Mouse siRNA
gguacgugcucagauaaagcug

SEQ ID NO: 292 - RNA, 22 nt, Mus musculus
  misc_feature (1)..(22): Mouse Passenger Strand Sequence
  misc_feature (21)..(22): n is a, c, g, or u
gcuuuaucugagcacguauunn

SEQ ID NO: 293 - RNA, 22 nt, Mus musculus
  misc_feature (1)..(22): Mouse siRNA
ggagcgcaguagcuauaugccu

SEQ ID NO: 294 - RNA, 22 nt, Mus musculus
  misc_feature (1)..(22): Mouse Passenger Strand Sequence
  misc_feature (21)..(22): n is a, c, g, or u
gcauauagcuacugcgcuuunn

SEQ ID NO: 295 - RNA, 22 nt, Mus musculus
  misc_feature (1)..(22): Mouse siRNA
gagcgcaguagcuauaugccuu

SEQ ID NO: 296 - RNA, 22 nt, Mus musculus
  misc_feature (1)..(22): Mouse Passenger Strand Sequence
  misc_feature (21)..(22): n is a, c, g, or u
ggcauauagcuacugcguuunn

SEQ ID NO: 297 - RNA, 22 nt, Mus musculus
  misc_feature (1)..(22): Mouse siRNA
uucccgcucgaaucaacaccaa

SEQ ID NO: 298 - RNA, 22 nt, Mus musculus
  misc_feature (1)..(22): Mouse Passenger Strand Sequence
  misc_feature (21)..(22): n is a, c, g, or u
gguguugauucgagcgggaann

SEQ ID NO: 299 - RNA, 22 nt, Mus musculus
  misc_feature (1)..(22): Mouse siRNA
gagcgaggaaaggggaggccag

SEQ ID NO: 300 - RNA, 22 nt, Mus musculus
  misc_feature (1)..(22): Mouse Passenger Strand Sequence
  misc_feature (21)..(22): n is a, c, g, or u
ggccuccccuuuccucguuunn

SEQ ID NO: 301 - DNA, 19 bp, Artificial Sequence
  Synthetic Primer -  PRRX1
gaaccgaagctgggagaaa

SEQ ID NO: 302 - DNA, 20 bp, Artificial Sequence
  Synthetic Primer -  PRRX1
aggagagagagagagagaga

SEQ ID NO: 303 - DNA, 22 bp, Artificial Sequence
  Synthetic Primer -  SNAI2
gcagacccactctgatgtaaag

SEQ ID NO: 304 - DNA, 22 bp, Artificial Sequence
  Synthetic Primer -  SNAI2
cagccagactcctcatctttat

SEQ ID NO: 305 - DNA, 23 bp, Artificial Sequence
  Synthetic Primer -  Col1a1
aggtgctatttaacaagggagaa

SEQ ID NO: 306 - DNA, 21 bp, Artificial Sequence
  Synthetic Primer -  Col1a1
agataggcccaggaactagaa

SEQ ID NO: 307 - DNA, 22 bp, Artificial Sequence
  Synthetic Primer -  Cepba
gagggactggagttatgacaag

SEQ ID NO: 308 - DNA, 20 bp, Artificial Sequence
  Synthetic Primer -  Cepba
tgggacacagagaccagata

SEQ ID NO: 309 - DNA, 20 bp, Artificial Sequence
  Synthetic Primer -  Ppia
gtggtctttgggaaggtgaa

SEQ ID NO: 310 - DNA, 22 bp, Artificial Sequence
  Synthetic Primer -  Ppia
gtcggagatggtgatcttcttg

SEQ ID NO: 311 - DNA, 20 bp, Artificial Sequence
  Synthetic Primer -  Runx2
gaagaaccaagtggcgagat

SEQ ID NO: 312 - DNA, 22 bp, Artificial Sequence
  Synthetic Primer -  Runx2
gtaaagacggtgatggtcagag

SEQ ID NO: 313 - DNA, 20 bp, Artificial Sequence
  Synthetic Primer -  Spp1
cagccaaggaccaactacaa

SEQ ID NO: 314 - DNA, 18 bp, Artificial Sequence
  Synthetic Primer -  Spp1
tgccaaactcagccactt

SEQ ID NO: 315 - DNA, 20 bp, Artificial Sequence
  Synthetic Primer -  Sox9
catcaagacggagcaactga

SEQ ID NO: 316 - DNA, 20 bp, Artificial Sequence
  Synthetic Primer -  Sox9
tgtagtgcggaaggttgaag

SEQ ID NO: 317 - DNA, 22 bp, Artificial Sequence
  Synthetic Primer -  Acan
ctggaagagaggacacagaaac

SEQ ID NO: 318 - DNA, 21 bp, Artificial Sequence
  Synthetic Primer -  Acan
ctcctgaagctgaggtctcta

SEQ ID NO: 319 - DNA, 22 bp, Artificial Sequence
  Synthetic Primer -  Myc
gagctggaatctcgagtgtaag

SEQ ID NO: 320 - DNA, 20 bp, Artificial Sequence
  Synthetic Primer -  Myc
agccaaggttgtgaggttag

SEQ ID NO: 321 - DNA, 22 bp, Artificial Sequence
  Synthetic Primer -  B2m
cagttccacccacctcaaatag

SEQ ID NO: 322 - DNA, 22 bp, Artificial Sequence
  Synthetic Primer -  B2m
gtgtgagccaggatgtagaaag

SEQ ID NO: 323 - DNA, 20 bp, Artificial Sequence
  Synthetic Primer -  Sox2
taagactagggctgggagaa

SEQ ID NO: 324 - DNA, 20 bp, Artificial Sequence
  Synthetic Primer -  Sox2
gccgcgattgttgtgattag

SEQ ID NO: 325 - DNA, 20 bp, Artificial Sequence
  Synthetic Primer -  Nanog
gaagatgcggactgtgttct

SEQ ID NO: 326 - DNA, 22 bp, Artificial Sequence
  Synthetic Primer -  Nanog
gctcaggttcagaatggtagag

SEQ ID NO: 327 - DNA, 22 bp, Artificial Sequence
  Synthetic Primer -  Klf4
gccaggagagagttcagtattt

SEQ ID NO: 328 - DNA, 20 bp, Artificial Sequence
  Synthetic Primer -  Klf4
ctgacttgctgggaacttga

(19) United States
(12) Patent Application Publication
(10) Pub. No.: US 2022/0356451 A1
(43) Pub. Date: Nov. 10, 2022

(54) METHODS TO REPROGRAM SOMATIC CELLS TO ALTERNATIVE CELL FATES OR PRIMITIVE CELL STATES

(71) Applicants: Tania Ray, Lincolnwood, IL (US); Partha S. Ray, Lincolnwood, IL (US)

(72) Inventors: Tania Ray, Lincolnwood, IL (US); Partha S. Ray, Lincolnwood, IL (US)

(21) Appl. No.: 17/597,776

(22) PCT Filed: Jul. 23, 2020
(86) PCT No.: PCT/US2020/043349

§ 371 Date: Jan. 21, 2022

Related U.S. Application Data
(60) Provisional application No. 62/877,798, filed on Jul. 23, 2019.

(51) Int. Cl. C12N 5/077; C12N 15/113

(52) U.S. Cl. CPC C12N 5/0656; C12N 15/113; C12N 2510/00

(57) ABSTRACT

Induced overexpression of defined exogenous transcription factors (TFs), or alternatively treatment with specific pathway modulatory cocktails, can reprogram somatic cells to pluripotency or alternate cell states. A barrier to initiating reprogramming lies in the starting cell's molecular identity, enforced by lineage-instructive TFs. However, it remained unclear whether repression of such somatic lineage-defining TFs of the starting cell in the absence of exogenous TFs is sufficient to induce cell reprogramming Using an intra-species somatic cell hybrid model, SNAI2 and PRRX1 were identified as the most critical determinants of mesenchymal commitment in rat embryonic fibroblasts (REFs) and demonstrate that siRNA-mediated transient knockdown of these individual factors is adequate to convert REFs into functional adipocytes, chondrocytes or osteocytes without requiring the provision of exogenous TFs. Additionally, it was shown that siRNA-mediated transient knockdown of SNAI2 alone, in the absence of exogenous TFs, is sufficient to transform REFs to a dedifferentiated pluripotent stem-like cell (dPSC) state that forms embryoid bodies and is capable of triple germ layer differentiation. These results establish for the first time that transient repression of a single somatic lineage-defining TF can effectively induce transdifferentiation to alternative somatic cell states or dedifferentiation to dPSCs in the absence of exogenous TFs or small molecule cocktails.

### CROSS-REFERENCE TO RELATED APPLICATIONS

This patent document claims priority to and benefits of U.S. Provisional Patent Application No. 62/877,798 entitled “METHODS TO REPROGRAM SOMATIC CELLS TO ALTERNATIVE CELL FATES OR PRIMITIVE CELL STATES” filed on Jul. 23, 2019. The entire content of the aforementioned patent application is incorporated by reference as part of the disclosure of this patent document.

### BACKGROUND

In 2006, researchers Takahashi and Yamanaka revolutionized stem cell research by showing that forced expression of only four transcription factors (Oct4, Sox2, Klf4, and c-Myc (OSKM) was sufficient to convert fibroblast cells into embryonic stem cell (ESC)-like cells, which they named induced pluripotent stem cells (iPSC). Induced pluripotent stem cells (iPSCs) hold tremendous promise for drug development, disease modelling and regenerative medicine since they can be generated in vitro, are able to differentiate into any cell types and are patient-specific and hence can eliminate tissue matching and rejection problems that currently hinder successful cell and tissue transplantation. Furthermore, iPSCs have no ethical concerns unlike ESCs because they are derived from somatic cells and not from developing embryos.

However, for these applications, the generated iPSCs should be pure, homogeneous, chromosomally stable and generated in appreciable yield and purity. The use of viral vectors and oncogenes like Myc as factors for OSKM mediated cell reprogramming has generated valid concerns over the safe use of the generated iPSC cells in clinics. Presently most iPSCs are generated either by ectopic expression of reprogramming factors delivered via viral vectors or by non-viral ways via transfection with messenger RNA (mRNA) instead of DNA (Warren et al. 2010) or treatment with small molecular cocktails/chemical reprogramming (Huangfu et al. 2008, Xu et al. 2008, Ichida et al. 2009).

A major drawback of both TF mediated reprogramming and chemical reprogramming happens to be the extremely low reprogramming efficiency. Previous studies have demonstrated that using the OSKM factors, an average somatic reprogramming efficiency of colony-forming pluripotent stem cells is around 0.2%-0.8%. (Anokye-Danso et al., 2011) Recently, the field of cell reprogramming has shifted towards the non-viral chemical/small molecule-based reprogramming strategy whereby, somatic cells are reprogrammed to pluripotency by treatment with pathway modulating small molecule cocktails. In 2013, Hou et al. demonstrated successful reprogramming of mouse cells into iPS cells for the first time using a combination of seven small molecules namely VPA (HDAC inhibitor), CHIR99021 (GSK3 inhibitor), RepSox E616452 (ALK5 inhibitor of TGF-Beta pathway), Tranylcypromine (Lysine specific demethylase inhibitor), Forskolin (cAMP signaling activator), 3-deazaneplanocin A DZNep (global histone methylation inhibitor) and TTNPB (Activates Retinoic Acid receptor RAR). Though path breaking, the yield of iPSCs generated using small molecules is still very low (efficiency around 0.2%).

Due to current limitations in the field, it would be desirable to produce methods for generating reprogrammable somatic cells at a high efficiency, without the need for ectopic expression of reprogramming factors or other cell reprogramming pathway modulators.

### SUMMARY

In some embodiments, a somatic fibroblast cell capable of transdifferentiating to a somatic cell of alternate lineage in the absence of viral or non-viral delivery of exogenous transcription factors and/or small molecule modulators is provided, the somatic fibroblast cell comprising an siRNA or shRNA molecule that inhibits expression of a single lineage defining transcription factor. In some aspects, the siRNA or shRNA molecule inhibits SNAI2, PRRX1, NAND1, CDX2, or a combination thereof. In some aspects, the somatic cell of alternate lineage is a somatic cell of adipocytic lineage, a somatic cell of osteogenic lineage, or a somatic cell of chondrogenic lineage.

In other embodiments, a somatic fibroblast cell capable of dedifferentiating to a multipotent or pluripotent stem cell in the absence of viral or non-viral delivery of exogenous transcription factors and/or small molecule modulators is provided; the somatic fibroblast cell is treated with siRNA or shRNA molecule that inhibits expression of a single lineage defining transcription factor. In some aspects, the siRNA or shRNA molecule inhibits SNAI2, PRRX1, NAND1, CDX2, or a combination thereof. In some aspects, the multipotent or pluripotent stem cell is a dedifferentiated multipotent mesenchymal stem cell or a dedifferentiated pluripotent stem cell.

In some embodiments, a population of transdifferentiated somatic cells is provided. The population of transdifferentiated somatic cells are generated from, a population of lineage committed fibroblast cells at an efficiency of more than 1%. In some aspects, the population of transdifferentiated somatic cells is a population of somatic cells of adipocytic lineage, a population of somatic cells of osteogenic lineage, or a population of somatic cells of chondrogenic lineage. In other aspects, the population of transdifferentiated somatic cells may be used in a tissue reconstruction procedure. For example, the population of transdifferentiated somatic cells may be used in a reconstructive plastic surgery procedure or a reconstructive orthopedic surgery procedure.

In other embodiments, a population of dedifferentiated stem cells is provided. The population of dedifferentiated stem cells includes a population of reprogrammed fibroblast cells lacking permanent genetic modification, wherein the population of reprogrammed fibroblast cells were induced to become the population of dedifferentiated stem cells from a population of adult fibroblast cells at an efficiency of more than 1%. In some aspects, the population of dedifferentiated stem cells is a population of dedifferentiated multipotent mesenchymal stem cells or a population of dedifferentiated pluripotent stem cells. In other aspects, the population of dedifferentiated stem cells may be used in a tissue reconstruction procedure, a wound healing application, or a transplantation procedure.

In some embodiments, a cell culture system for producing a population of transdifferentiated somatic cells is provided. The cell culture system may include a population of lineage committed somatic cells, an siRNA or shRNA molecule that inhibits expression of a single lineage defining transcription factor; and a culture medium comprising a cell culture media specific to the population of transdifferentiated somatic cells, wherein the cell culture system does not include an exogenous transcription factor and/or small molecule modulator (chemical reprogramming). In certain aspects, the population of lineage committed somatic cells of the cell culture system are fibroblast cells. In certain aspects, the single lineage defining transcription factor is SNAI2, PRRX1, HAND1, CDX2, or a combination thereof. In certain aspects, the population of transdifferentiated somatic cells are of adipocytic lineage, osteogenic lineage, or chondrogenic lineage.

In other embodiments, a cell culture system for producing a population of dedifferentiated stem cells is provided. The cell culture system may include a population of lineage committed somatic cells, an siRNA or shRNA molecule that inhibits expression of a single lineage defining transcription factor, and a culture medium comprising a stem cell media specific to the population of dedifferentiated stem cells, wherein the cell culture system does not include an exogenous transcription factor and/or small molecule modulator. In certain aspects, the population of somatic cells of the cell culture system are fibroblast cells. In certain aspects, the single lineage defining transcription factor is SNAI2, PRRX1, HAND1, CDX2, or a combination thereof. In certain aspects, the population of dedifferentiated stem cells is a population of dedifferentiated multipotent mesenchymal stem cells or a population of dedifferentiated pluripotent stem cells.

In other embodiments, a method for producing a transdifferentiated somatic cell is provided. The method may include the following steps: introducing an siRNA or shRNA molecule that transiently inhibits expression of a single lineage defining transcription factor of a lineage committed somatic cell; and incubating the somatic cell in a culture media specific to the transdifferentiated somatic cell; wherein the method for producing the transdifferentiated somatic cell is performed in the absence of viral or non-viral delivery of exogenous transcription factors and/or small molecule modulators (chemical reprogramming). In certain aspects, the population of somatic cells of the cell culture system are lineage committed fibroblast cells. In certain aspects, the single lineage defining transcription factor is SNAI2, PRRX1, HAND1, CDX2, or a combination thereof. In certain aspects, the transdifferentiated somatic cell is a cell of adipocytic lineage, osteogenic lineage, or chondrogenic lineage.

In other embodiments, a method for producing a dedifferentiated stem cell is provided. The method may include the following steps: introducing an siRNA or shRNA molecule that transiently inhibits expression of a single lineage defining transcription factor of a lineage committed somatic cell; and incubating the siRNA/shRNA treated cell in a culture media specific to the dedifferentiated stem cells (Mesenchymal stem cells or Embryonic stem cells); wherein the method for producing the dedifferentiated stem cell is performed in the absence of viral or non-viral delivery of exogenous transcription factors and/or small molecule modulators (chemical reprogramming). In certain aspects, the population of lineage committed somatic cells of the cell culture system are fibroblast cells. In certain aspects, the single lineage defining transcription factor is SNAI2, PRRX1, NAND1, CDX2, or a combination thereof. In certain aspects, the dedifferentiated stem cell is a dedifferentiated multipotent mesenchymal stem cell or a dedifferentiated pluripotent stem cell.

In some embodiments, an autologous tissue graft is provided. The autologous tissue graft includes a population of transdifferentiated somatic cells, wherein (i) the population of transdifferentiated somatic cells is derived from a population of somatic cells obtained from a subject for use in a tissue reconstruction procedure, and (ii) the population of transdifferentiated somatic cells is generated from a population of siRNA treated fibroblast cells lacking permanent genetic modification (as described in 0011 above), wherein the population of reprogrammed fibroblast cells were induced to become the population of transdifferentiated somatic cells at an efficiency of more than 1%. In some aspects, the population of transdifferentiated somatic cells is produced using the methods described in the embodiments above.

In other embodiments, an autologous tissue graft is provided. The autologous tissue graft includes a population of dedifferentiated stem cells, wherein (i) the population of dedifferentiated stem cells is derived from a population of somatic cells obtained from a subject for use in a tissue reconstruction procedure, a wound healing application, or a transplantation procedure, and (ii) the population of dedifferentiated stem cells is generated from a population of reprogrammed fibroblast cells lacking permanent genetic modification (as described in 0012 above), wherein the population of reprogrammed fibroblast cells were induced to become the population of dedifferentiated stem cells at an efficiency of more than 1%. In some aspects, the population of dedifferentiated stem cells is produced using the methods described in the embodiments above.

In some embodiments, a method for treating a condition in a subject is provided. The method may include a step of grafting or transplanting a population of reprogrammed somatic cells, dedifferentiated stem cells, or transdifferentiated somatic cells into or onto a tissue or organ of the subject. Examples of cells and conditions that may be treated include, but are not limited to those described throughout the disclosure. In some embodiments, the population of reprogrammed somatic cells, dedifferentiated stem cells, or transdifferentiated somatic cells used in the method for treating a condition are part of an autologous tissue graft.

### BRIEF DESCRIPTION OF THE DRAWINGS

This application contains at least one drawing executed in color. Copies of this application with color drawing(s) will be provided by the Office upon request and payment of the necessary fees.

FIGS. 1A-1D show the identification of core transcriptional regulators that define mesenchymal fibroblast identity.

FIG. 1A shows a Venn diagram representation of the two-step bioinformatic strategy to identify core transcriptional regulators of mesenchymal fibroblast cell identity. By selecting transcription factors (TFs) that displayed a >5-fold expression in rat embryonic fibroblast (REF) cells of mesenchymal origin when compared to rat hepatoma cell (RH) of endodermal origin, 149 TFs were grouped as mesenchymal fibroblast-enriched TFs (MFEFs). Out of these 149 MFEFs, 86 TFs that displayed >2.5-fold repression in the intra-species hepatoma-fibroblast synkaryon cell hybrid (HF) when compared to REFs, were identified as mesenchymal fibroblast-specific TFs (MFSFs).

FIG. 1B shows a gene expression microarray heatmap of REF, RH, and HF cell lines showing all genes that displayed >5 fold overexpression in REF compared to RH cells. TF gene-members were shortlisted as MFEFs.

FIG. 1C shows a gene expression microarray heat map of HF and REF cell lines showing all genes that displayed >2.5-fold repression in HF when compared to REFs. TF gene-members were identified as MFEFs.

FIG. 1D is a list of top ten MFEFs and MFEFs are tabulated. It is noted that the rank order of the top MFEFs changed in the core MFEFs list, after applying the two-step selection strategy.

FIGS. 2A-2E show the effect of engineered re-expression of repressed fibroblast-specific transcription factors (TFs) in somatic hybrids.

FIG. 2A is a schematic diagram depicting the morphologic transformation by cell fusion of rat hepatoma (RH) cells of endodermal origin and rat embryonic fibroblasts (REFs) of mesenchymal origin to generate stable intra-species hepatoma fibroblast (HF) synkaryon hybrid cells, and the subsequent transformation of HF cells by engineered overexpression of PRRX1 and SNAI2, respectively.

FIG. 2B shows a series of photos as indicated. Starting from the top row, Row 1 of FIG. 2B is a series of photomicrographs (brightfield) that show round morphology of RH clusters, spindle shaped morphology of REF, and hybrid morphology of HF. Re-acquisition of spindle-shaped elongated morphology in G418-resistant PRRX1 overexpressed HF clone (HF-PRRX1) and G418-resistant SNAI2 overexpressed HF clone (HF-SNAI2). (Scale bar, 10 μm). Row 2 of FIG. 2B is a series of immunofluorescent staining pictures of prototypical fibroblast-specific marker Collagen1a1 (Col1a1) in rat hepatoma cells (RH), rat embryonic fibroblast (REF) cells, rat hepatoma-fibroblast hybrid (HF) cells, G418-resistant PRRX1 overexpressed HF clone (HF-PRRX1) and G418-resistant SNAI2 overexpressed HF clone (HF-SNAI2). Col1a1 expression is significantly higher in (REF) cells, in comparison to RH and HF cells. Upon engineered re-expression of SNAI2 and PRRX1 in HF, Col1a1 expression significantly increases (Col1a1 stained in red and DAPI in blue) (Scale bar, 10 μm). Row 3 of FIG. 2B is a series of immunofluorescent staining pictures showing PRRX1 expression in RH cells, REF cells, HF cells, HF-PRRX1 cells and HF-SNAI2 cells. (Scale bar, 10 μm). Row 4 of FIG. 2B is a series of immunofluorescent staining pictures of SNAI2 expression in RH cells, REF cells, HF cells, HF-PRRX1 cells and HF-SNAI2 cells (SNAI2 is stained in green and DAPI in blue). (Scale bar, 10 μm).

FIG. 2C is a series of bar graphs showing fold repression of PRRX1, SNAI2 and Col1a1 in HF Hybrids compared to REFs as validated by qRT-PCR (Top), re-establishment of prototypical mesenchymal fibroblast TF and gene expression upon engineered re-expression of repressed fibroblast-specific TFs PRRX1 and SNAI2 (Middle and Bottom, respectively).

FIG. 2D is a bar graph that depicts the TGF Beta-responsive cell migration ability of different cell lines. (REF) cells show the highest migration ability in response to TGF Beta stimulation when compared to (RH) hepatoma or (HF) hybrids cells, the G418-resistant PRRX1 overexpressed HF clone (HF-PRRX1) and G418-resistant SNAI2 overexpressed HF clone (HF-SNAI2) show significant re-acquisition of TGF Beta-responsive cell migration ability, a prototypical fibroblast-specific functional trait.

FIG. 2E shows a schematic diagram depicting transdifferentiation of REFs into adipocytes, osteocytes, chondrocytes and dedifferentiation into mesenchymal stem cells on individual treatment with siSnai2 or siPRRX1. dPSCs were generated only in the siSnai2 group.

FIGS. 3A-3J show that siRNA mediated transient repression of SNAI2 or PRRX1 in Rat Embryonic Fibroblasts (REFs) induces ex vivo adipocytic, osteocytic or chondrocytic transdifferentiation.

FIG. 3A shows a series of photos as indicated. Starting from the top row, the left column of Row 1 of FIG. 3A shows brightfield photomicrographs of REFs transfected with siCntrl that were then incubated in in adipogenic media for 14 days. The left column of Row 2 of FIG. 3A shows brightfield photomicrographs of REFs transfected with siPRRX1 that were then incubated in adipogenic media for 14 days. The left column of Row 3 of FIG. 3A shows brightfield photomicrographs of REFs transfected with siSnai2 that were then incubated in adipogenic media for 14 days. Adjacent IF photomicrographs in each Row assess expression of adipogenic TF Cepba in Red and DAPI nuclear counterstain in Blue (Scale bar, 10 μm) in all three groups (siCntrIREF, siPRRX1REF and siSnai2REF). siSnai2REF group displayed highest adipogenesis and accumulation of fat droplets filled vacuoles as ascertained by Oil Red 0 staining.

FIG. 3B shows a series of photos as indicated. Starting from the top row, the left column of Row 1 of FIG. 3A shows brightfield photomicrographs of REFs transfected with siCntrl that were then incubated in osteogenic media for 14 days. The left column of Row 2 of FIG. 3B shows brightfield photomicrographs of REFs transfected with siPRRX1 that were then incubated in osteogenic induction media for 14 days. The left column of Row 3 of FIG. 3B shows brightfield photomicrographs of REFs transfected with siSnai2 that were then incubated in osteogenic media for 14 days. Adjacent IF photomicrographs in each Row assess expression of osteogenic TF Runx2 in (Red and DAPI nuclear counterstain in Blue) in all three groups (siCntrIREF, siPRRX1REF and siSnai2REF). (Scale bar, 10 μm). siPRRX1REF group displayed highest osteogenesis and mineralized bone deposition as ascertained by Alizarin red staining.

FIG. 3C shows a series of photos as indicated. Starting from the top row, the left column of Row 1 of FIG. 3C shows brightfield photomicrographs of REFs transfected with siCntrl in chondrogenic media for 14 days. The left column of Row 2 of FIG. 3C shows brightfield photomicrographs of REFs transfected with siPRRX1 in chondrogenic media for 14 days. The left column of Row 3 of FIG. 3C shows brightfield photomicrographs of REFs transfected with siSnai2 that were then incubated in chondrogenic media for 14 days. Adjacent IF photomicrographs in each row assess expression of chondrogenic TF Sox9 in Red and DAPI nuclear counterstain in Blue in all three groups (siCntrIREF, siPRRX1REF and siSnai2REF). (Scale bar, 10 μm). siSnai2REF cells displayed highest chondrogenesis and aggrecan formation as ascertained by Alcian blue staining.

FIG. 3D is a bar graph showing the % intensity of Oil Red O staining to ascertain adipogenesis as quantitated by Image J software analysis.

FIG. 3E is a bar graph showing the % intensity of Alizarin Red staining to ascertain osteogenesis as quantitated by Image J software analysis.

FIG. 3F is a bar graph showing the % intensity of Alcian Blue staining to ascertain chondrogenesis as quantitated by Image J software analysis.

FIG. 3G is a series of bar graphs showing the results of a qRT-PCR analysis, which shows increase in fold expression of mRNA of adipocyte specific TF Cebpa in siPRRX1 and siSnai2REFs incubated in adipogenic media for 14 days. Expression of each target gene was calculated as a relative expression to house-keeping gene peptidyl prolylisomerase (Ppia) and represented as fold induction over control REF cells treated with siCntrl. Data are represented as mean±Standard deviation (SD) of 3 independent experiments.

FIG. 3H is a series of bar graphs showing the results of a qRT-PCR analysis, which shows an increase in fold expression of mRNA of osteocyte specific TF Runx2 in siPRRX1 and siSnai2 treated REFs that were then incubated in osteogenic media for 14 days (Left), Expression of each target gene was calculated as a relative expression to housekeeping gene secreted phosphor protein1 (Spp1) and represented as fold induction over control cells treated with siCntrl. Data are represented as mean±SD of 3 independent experiments.

FIG. 3I is a series of bar graphs showing the results of a qRT-PCR analysis, which shows an increase in fold expression of mRNA of chondrocyte specific TF Sox9 in siPRRX1 and siSnai2 treated REFs incubated in chondrogenic media for 14 days (Left), Expression of each target gene was calculated as a relative expression to housekeeping gene peptidylprolyl isomerase (Ppia) and represented as fold induction over control cells treated with siCntrl. Data are represented as mean±SD of 3 independent experiments.

FIG. 3J is a schematic diagram illustrating the process of siRNA-mediated cellular reprogramming in the absence of exogenous factors.

FIG. 4 Dedifferentiation of Rat Embryonic Fibroblasts (REFs) to dMSCs and dPSCs by transient repression of SNAI2 and PRRX1.

FIG. 4A is a series of brightfield photomicrographs of REFs transfected with siCntrl (Row1) siPrrxi (Row2) and siSnai2 (Row3). siPRRX1REFs and siSnai2REFs on incubation in Mesenchymal stem cell media for 14 days resulted in generation of dedifferentiated Mesenchymal Stem-like cells (dMSCs). Adjacent IF photomicrographs assess expression of mesenchymal stem cell specific TF Myc in Red and DAPI nuclear counterstain in Blue (Scale bar, 10 μm) in the three groups.

FIG. 4B is a bar graph showing MSC generation, which was ascertained using Alkaline Phosphatase (ALP) Assay. siSnai2REF cells showed the highest mesenchymal stem cell activity.

FIG. 4C is a series of bar graphs showing the results of a real-time qPCR analysis of steady state gene expression of MSC/dMSC genes. Expression of characteristic MSC TF Myc was calculated as a relative expression to beta-2-microglobulin (B2M) and represented as fold induction over siCntrl transfected cells. Data are represented as mean±SD of 3 independent experiments.

FIG. 4D is a series of brightfield photomicrographs of REFs transfected with siCntrl (Row1), siPrrxi (Row2), and siSnai2 (Row3). REFs on incubation in Rat ESC media for 14 days resulted in generation of dedifferentiated Pluripotent Stem-like cells (dPSCs). Brightfield photomicrographs of free floating embryoid body formation when dPSCs were suspended in differentiation media in low attachment plates for 8 days. Only the siSnai2 transfected REFs underwent effective nuclear reprogramming to dPSC cells as evidenced by their ability to form embryoid bodies in suspension culture (Row 3). Adjacent IF photomicrographs assess expression of ESC TFs Sox2, and Nanog in Red and DAPI nuclear counterstain in Blue for all three groups. (Scale bar, 10 μm).

FIG. 4E is a series of bar graphs showing the results of a qRTPCR analysis of steady state gene expression of characteristic ESC TFs Sox2 and Nanog and Klf4 were calculated as a relative expression to beta-2-microglobulin (B2M) and represented as fold induction over siCntrl transfected cells. Only the siSnai2 transfected REFs showed expression of pluripotency TFs Sox2 and Nanog. Data are represented as mean±SD of 3 independent experiments.

FIG. 4F is a series of photomicrographs of the dPSC-derived Day 8 embryoid bodies immunostained for Ectodermal TFs. Ectoderm differentiated cells were simultaneously stained with Northern Lights™ (NL) 557-conjugated OTX-2 (red) and NL493-conjugated SOX2 (green) with DAPI (blue) nuclear counterstain.

FIG. 4G is a series of photomicrographs of the dPSC-derived Day 8 embryoid bodies immunostained for Mesodermal TFs. Mesoderm differentiated cells were simultaneously stained with NL557-conjugated Brachyury (green) and NL637-conjugated Hand1 (red) with DAPI (blue) nuclear counterstain.

FIG. 4H is a series of photomicrographs of the dPSC-derived Day 8 embryoid bodies immunostained for Endodermal TFs. Endoderm differentiated cells were simultaneously stained with NL637-conjugated Sox1 7 (red) and NL493-conjugated Gata-4 (green). All nuclei were stained with DAPI (blue).

FIG. 5 is a flow chart showing generation of somatic cell hybrids.

FIG. 6 is a model for generating somatic cell hybrids. Indicated cell types were fused using polyethylene glycol and hybrids (FR) selected using medium that allows only hybrid cells to survive.

FIG. 7 is a series of data showing a comparative analysis of the degree of colocalization of SNAI2, HAND1 and CDX2 binding to genome-wide MFSG active enhancers and ESCG repressors in human mesoderm.

FIG. 7A is a Venn Diagram representation of overlap of active enhancers (as defined by colocalization of H3K4Me1 and H3K27Ac histone marks), SNAI2, HAND1 and CDX2 genomic targets that colocalize with defined active enhancers, and MFSG active enhancer targets in human mesoderm.

FIG. 7B is a bar graph showing the combined and individual percent colocalization of SNAI2, HAND1 and CDX2 genomic targets with MFSG active enhancer targets in human mesoderm.

FIG. 7C is a Venn Diagram representation of overlap of active enhancers (as defined by colocalization of H3K4Me1 and H3K27Ac histone marks), SNAI2, HAND1 and CDX2 genomic targets that colocalize with defined active enhancers, and MFSF active enhancer targets in human mesoderm.

FIG. 7D is a bar graph showing the combined and individual percent colocalization of SNAI2, HAND1 and CDX2 genomic targets with MFSF active enhancer targets in human mesoderm.

FIG. 7E is a Venn Diagram representation of overlap of repressed enhancers (as defined by colocalization of H3K4Me1 and H3K27me3 histone marks), SNAI2, HAND1 and CDX2 genomic targets that colocalize with defined repressed enhancers, and ESCG active repressed enhancer targets in human mesoderm.

FIG. 7F is a bar graph showing the combined and individual percent colocalization of SNAI2, HAND1 and CDX2 genomic targets with ESCG repressed enhancer targets in human mesoderm.

FIG. 7G is a Venn Diagram representation of overlap of repressed enhancers (as defined by colocalization of H3K4Me1 and H3K27me3 histone marks), SNAI2, HAND1 and CDX2 genomic targets that colocalize with defined repressed enhancers, and ESCF active repressed enhancer targets in human mesoderm.

FIG. 7H is a bar graph showing the combined and individual percent colocalization of SNAI2, HAND1 and CDX2 genomic targets with ESCF repressed enhancer targets in human mesoderm.

FIG. 8 shows the coincident shift in enhancer chromatin modifications that occur at SNAI2 active enhancer targets during mesoderm lineage progression.

FIG. 8A shows H3K4me1, H3K27ac and H3K27me3 ChIP-Seq tracks and matched RNA-Seq track at prototypical SNAI2-bound active enhancer MFSG target PRRX1 across the spectrum of mesodermal lineage progression acrossin 21 different human cells and tissues, from ESC to adult differentiated fibroblast, osteoblast, chondrocyte and adipocyte.

FIG. 8B shows H3K4me1, H3K27ac and H3K27me3 ChIP-Seq tracks and matched RNA-Seq track at prototypical SNAI2-bound active enhancer MFSG target FOXF1 across the spectrum of mesoderm lineage progression acrossin 21 different human cells and tissues, from ESC to adult differentiated fibroblast, osteoblast, chondrocyte and adipocyte.

FIG. 9 shows the coincident shift in enhancer chromatin modifications that occur at SNAI2 repressed enhancer targets during mesoderm lineage progression.

FIG. 9A shows H3K4me1, H3K27ac and H3K27me3 ChIP-Seq tracks and matched RNA-Seq track at prototypical SNAI2-bound repressed enhancer ESCG target SOX2 across the spectrum of mesodermal lineage progression acrossin 21 different human cells and tissues, from ESC to adult differentiated fibroblast, osteoblast, chondrocyte and adipocyte.

FIG. 9B shows H3K4me1, H3K27ac and H3K27me3 ChIP-Seq tracks and matched RNA-Seq track at prototypical SNAI2-bound repressed enhancer ESCG target ZIC2 across the spectrum of mesoderm lineage progression acrossin 21 different human cells and tissues, from ESC to adult differentiated fibroblast, osteoblast, chondrocyte and adipocyte.

FIG. 10 is a series of data showing SNAI2 enhancer binding in human mesoderm regulates transcriptional expression of MFSG active enhancer targets and repression of ESCG repressed enhancer targets. Data shown in FIG. 10 were visualized using the Washington University Genome Browser.

FIG. 10A is a Venn Diagram representation of overlap of active enhancers (as defined by colocalization of H3K4Me1 and H3K27Ac histone marks), SNAI2-bound active enhancers and MFSG targets.

FIG. 10B is a Venn Diagram representation of overlap of repressed enhancers (as defined by colocalization of H3K4me1 and H3K27me3 histone marks), SNAI2-bound repressed enhancers and ESCG targets.

FIG. 10C is a heat map showing hierarchical clustering of the RNA-Seq expression profile of the SNAI2-bound MFSG active enhancer targets across the spectrum of mesodermal lineage progression in 21 different human cells and tissues, from ESCs to adult differentiated fibroblasts.

FIG. 10D is a heat map showing hierarchical clustering of the RNA-Seq expression profile of the SNAI2-bound ESCG repressed enhancer targets across the spectrum of mesodermal lineage progression in 21 different human cells and tissues, from ESCs to adult differentiated fibroblasts.

FIG. 10E shows aggregate plots of H3K4me1, H3K27ac, H3K27me3 and H3K9me3 ChIP-Seq signals centered on the SNAI2 peak midpoint at identified active enhancer MFSG targets.

FIG. 10F shows example ChIP-Seq profiles of a prototypical SNAI2-bound active MFSG enhancer target gene, PRRX1 in human mesoderm. Data were visualized using the Washington University Genome Browser.

FIG. 10G shows aggregate plots of H3K4me1, H3K27ac, H3K27me3 and H3K9me3 ChIP-Seq signals centered on the SNAI2 peak midpoint at identified repressed enhancer ESCG targets. and

FIG. 10H shows example ChIP-Seq profiles of a prototypical SNAI2-bound repressed ESCG enhancer target gene, SOX2 in human mesoderm.

### DETAILED DESCRIPTION

Transdifferentiated and dedifferentiated cells and populations, reprogrammed cells, and methods for producing and using said cells are provided herein. Direct reprogramming of cells to desired lineages from easily available somatic cells has enormous potential in cell based regenerative therapies. Since the discovery of transcription factor (TF)-mediated reprogramming of differentiated somatic cells into induced pluripotent stem cells (iPSCs) by forced overexpression of Oct4-Sox2-Klf4-Myc (OSKM) (Takahashi & Yamanaka 2006), the field of somatic cell reprogramming has burgeoned into a hotbed of research, with several studies further expanding this work (Graf 2011; Srivastava & DeWitt 2016). Many groups have also reported direct programming of somatic cells into alternate differentiated cell types (i.e., transdifferentiation) by forced expression of lineage-instructive TFs (Davis et al. 1987; Vierbuchen et al. 2010; Xu eta al 2015). More recently, small molecule driven chemical reprogramming of somatic cells has been demonstrated to be an effective non-viral reprogramming strategy (Hou et al. 2013, Liu et al. 2016; Xie et al. 2017). However, despite significant advances, current methods for reprogramming lineage committed cells into iPSCs or other cell types often generate incompletely transformed cells due to retention of the starting cell's transcriptomic and epigenetic state.

Some studies have enhanced efficiency of exogenous TF-induced reprogramming using alternate methodologies (Weltner et al. 2018; Kogut et al. 2018),

But the difference between current approaches and the methods described herein lies in the fundamental strategy behind cell reprogramming. Current approaches focus on the characteristics of the target cell in an attempt to induce or “force” a lineage committed somatic cell to become another cell type, whereas the methods described herein focus on the starting cell to passively induce reprogramming. To accomplish this passive reprogramming, the methods described herein employ steps for repressing lineage instructive TFs specific to the starting or parental cell (also referred to herein as “parental TFs” or “lineage defining transcription factors”) alone to increase cellular plasticity and enable transdifferentiation to alternate cell fates or dedifferentiation to primitive cell states in the absence of transcription factors (TFs) or small molecule modulators specific to the target transdifferentiated or dedifferentiated cell (referred to herein as “exogenous TFs and/or modulators”). None of the studies reported thus far are known to have explored the potential of this strategy. And, no process to date has been reported wherein perturbation of a single gene in a differentiated cell, such as a fibroblast, can produce any cell of choice simply by altering culture conditions following the single gene perturbation.

The methods described herein are based on lifting restrictions imposed by lineage instructive TFs. Lineage-specific TFs of differentiated cells strictly enforce cell identity, resist plasticity acquisition and contribute to low reprogramming efficiency associated with exogenous TF-induced and/or small molecule-induced cellular reprogramming. Because the presence of residual epigenetic memory enforced by lineage-specific TFs of the starting cell is an impediment to nuclear reprogramming, lifting these restrictions imposed by such TFs permits reprogramming to proceed unhindered, even in the absence of Yamanaka factors or pathway modulatory cocktails.

In the studies described below, it was also shown for the first time that transient repression of a single lineage defining TF of a somatic cell can effectively overcome molecular safeguards preserving cell identity and is sufficient to induce its dedifferentiation to pluripotent cell states. Using this distinctive approach, pluripotent stem cells and cells of other lineages from lineage committed cells were generated. This contribution is significant because it establishes a new and effective in vitro reprogramming technique to generate dedifferentiated stem cells (including iPSCs) in good yield and purity, by overcoming molecular barriers that limit reprogramming efficiency and contribute to the emergence of partially reprogrammed cell populations without the need to ectopically overexpress pluripotency factors. Since this strategy does not require forced overexpression of ectopic genes like Oct4, Sox2, Klf4, c-Myc, dedifferentiated stem cells generated by the methods described herein will not harbor harmful oncogenic mutations. Moreover, because the methods are non-viral, they will not entail the risk associated with genomic integration of lentiviral or retroviral vectors. Additionally, the methods described herein will help improve the efficiency of existing iPSCs generation protocols. Improving efficiency of existing cell reprogramming techniques remains a critical unaddressed problem. The methods of generating dedifferentiated stem cells with improved efficiency described herein will eventually help accelerate use of iPSCs for therapeutic purposes.

Generation of Somatic Cell Hybrids and Identification of Lineage Defining Transcription Factors

The embodiments described herein relate to a method to reprogram lineage committed cells to a dedifferentiated or transdifferentiated state without introducing any exogenous TFs and/or modulators. This method is based on first identifying pivotal germ layer-specific lineage-instructive intrinsic factors (also referred to herein as “gatekeeper transcription factor” “parental TFs” or “lineage defining transcription factors” or “LDTF”)—which are responsible for maintaining the lineage commitment of a particular cell. To be an LDTF or gatekeeper transcription factor (GTF), the singular targeted inhibition of a factor should be capable of reversing lineage preservation and maintenance, and restoring multipotent and/or pluripotent differentiation capability. Furthermore, the targeted siRNA-mediated transient inhibition of the identified LDTF in differentiated cells should be a viable alternative to the forced overexpression of pluripotency factors in such cells as a way to generate multipotent and/or pluripotent stem cells from them without the need for permanent genetic modification. As disclosed below, LDTFs whose physiologic expression is important for inhibiting cell plasticity and pluripotency and safeguard the processes of lineage specification, commitment and preservation in differentiated cells were identified.

LDTFs may be identified using an unbiased, comprehensive experimental discovery approach involving generation of stable intra-species somatic cell synkaryon hybrids (HFs). The advantage of this technique is that in the generated hybrid cells, lineage instructive factors of the parental cells are repressed thus giving us a functional read out of the importance of these factors in determining the fate of the parental cells. Generation of HFs for use in identifying LDTFs for use in the embodiments described herein is detailed below.

In certain embodiments, LDTFs that may be used or modulated to reprogram lineage committed fibroblast cells in accordance with the embodiments described herein include, but are not limited to, SNAI2, PRRX1, NAND1, CDX2, or a combination thereof. Other LDTFs may be identified for other types of lineage committed somatic cells using a desired starting cell and employing the methodology described herein.

As such, a method to identify pivotal germ layer-specific LDTFs responsible for maintaining lineage commitment of a particular cell and whose transient repression can successfully affect nuclear reprogramming of such cells to their pre-lineage commitment state of cellular plasticity is provided. That method includes a step of generating a somatic cell hybrid by fusing two intra-species parental cells: a primary parental cell and a secondary parental cell. For purposes of this method, the primary parental cell refers to the cell for which identification of an LDTF is desired and for which nuclear reprogramming is desired. In certain embodiments, the primary parental cell is a lineage committed fibroblast cell, and the secondary cell is a lineage committed hepatoma cell, but any suitable somatic cells may be used as the primary and secondary parental cells.

To ease the transition between a lineage committed somatic cell and a transdifferentiated cell, it may be desirable to identify LDTFs in primary parental cell types derived from each of the primary germ layers. It is also desirable to use cells that can be obtained in a noninvasive matter as the primary cell type. For example, in certain embodiments, a fibroblast—which is derived from the mesoderm—is used as a primary parental cell to generate a somatic cell hybrid for identifying LDTFs in the fibroblast. LDTFs that are identified in the fibroblast may then be targeted to reprogram the fibroblast and generate a transdifferentiated somatic cell type derived from the mesoderm. In other embodiments, a keratinocyte—which is derived from the ectoderm—is used as a primary parental cell to generate a somatic cell hybrid for identifying LDTFs in the keratinocyte. LDTFs that are identified in the keratinocyte may then be targeted to reprogram the keratinocyte and generate a transdifferentiated somatic cell type derived from the ectoderm. And, in other embodiments, an endothelial cell from the gut lining or a hepatocyte—which are derived from the endoderm—is used as a primary parental cell to generate a somatic cell hybrid for identifying LDTFs in the gut lining endothelial cell or hepatocyte. LDTFs that are identified in the gut lining endothelial cell or hepatocyte may then be targeted to reprogram the gut lining endothelial cell or hepatocyte and generate a transdifferentiated somatic cell type derived from the endoderm.

The somatic cell hybrid and each of the parental cells are each subjected to a genome-wide transcriptomic profiling technique to identify LDTFs that enforce cell fate in both parental cells and in the somatic cell hybrid. The genome-wide transcriptomic profiling technique may be a suitable technique in the art including, but not limited to, whole genome microarrays, DNA sequencing, RNA sequencing, chromatin profiling, and chromatin immunoprecipitation (ChIP) assays, and any other Next generation sequencing (NGS) technique known in the art.

Identification of candidate LDTFs involves selecting a set of primary TFs that demonstrate greater than 5-fold overexpression in the primary parental cell as compared to the secondary parental cell. The primary TFs are then screened to identify a set of hybrid TFs that demonstrate greater than 2.5-fold repression in the somatic cell hybrid as compared to the primary parental cell. The hybrid TFs are then used as candidate LDTFs to target in the primary parental cell type. In certain embodiments, the hybrid TFs that demonstrate the greatest repression are selected as target LDTFs. In the case where the primary parental cell is a lineage committed fibroblast cell, the target LDTFs selected were SNAI2, PRRX1, NAND1, CDX2, or a combination thereof according to certain embodiments described herein. However, this method may be applied to any primary parental cell to be used as a starting cell for the reprogramming methods described herein.

Generation of Transdifferentiated Cells

According to the embodiments described herein, identification of LDTFs as described above and in the working examples and treatment with LDTF specific siRNA allows for the generation of transdifferentiated or dedifferentiated cells without genetic modification of the starting somatic cells by forced expression of reprogramming factors, without non-viral delivery of exogenous reprogramming factors and without treatment with small molecule pathway modulators. In other words, the methods provided herein provide for the generation of transdifferentiated or dedifferentiated cells in the absence of viral- or non-viral delivery of exogenous reprogramming factors and small molecule pathway modulators.

In one embodiment, a method for producing a transdifferentiated somatic cell is provided. In another embodiment, a method for producing a dedifferentiated stem cell is provided. Both of these methods may include a step of transient inhibition (or repression) of an LDTF in a lineage committed somatic cell. In some embodiments, the transient inhibition step is performed in a lineage committed fibroblast cell. In such embodiments, the LDTF or LDTFs that are transiently inhibited may be (i) SNAI2, (ii) PRRX1, (iii) HAND1, (iv) CDX2, (v) SNAI2 and PRRX1, (vi) SNAI2 and HAND1, (vii) SNAI2 and CDX2, or (viii) SNAI2, HAND1, and CDX2.

Transient inhibition of an LDTF is accomplished by introducing any molecule able to temporarily suppress its expression. In one embodiment, transient inhibition of the LDTF is accomplished by the use of an siRNA or shRNA molecule. While other types of molecules and systems may be used to transiently inhibit the LDTF (e.g., antisense oligonucleotides, miRNA), siRNA and shRNA molecules are advantageous because they are designed to target and inhibit a specific gene, can reduce gene expression at the mRNA level, and can be delivered to cells non-virally. In other words, siRNA and shRNA molecules can selectively and temporarily inhibit a single target gene with minimal off-target effects using the RNA interference mechanism in the cell. Other small molecules such as miRNA have multiple targets, thus limiting their selectivity. Permanent solutions for silencing a single gene (e.g., CRISPR Cas9) are also not preferred because permanent gene knockout could be detrimental to the dynamic process of cell reprogramming, and likely does not work. In accordance with such an embodiment, the method of producing a transdifferentiated somatic cell includes a step of introducing an siRNA or shRNA molecule that transiently inhibits expression of an LDTF—and in certain aspects, inhibits expression of a single LDTF—of the lineage committed somatic cell. In other words, lineage committed somatic cells are treated with the siRNA or shRNA molecule targeting an LDTF and incubated in the media of the desired target cell in order to transdifferentiate them to alternate cell fates or dedifferentiate them to multipotent or pluripotent primitive cell states.

In an embodiment where the lineage committed somatic cell is a fibroblast cell, the LDTF that is transiently inhibited by the siRNA or shRNA molecule is (i) SNAI2, (ii) PRRX1, (iii) HAND1, (iv) CDX2, (v) SNAI2 and PRRX1, (vi) SNAI2 and HAND1, (vii) SNAI2 and CDX2, or (viii) SNAI2, HAND1, and CDX2. In certain aspects, the LDTF that is transiently inhibited by the siRNA or shRNA molecule is SNAI2 alone, PRRX1 alone, HAND1 alone, or CDX2 alone (i.e., transient inhibition of a single LDTF).

In one embodiment, an siRNA molecule used to transiently inhibit the LDTF may be derived from an shRNA delivered to the cell and subsequently cleaved by Dicer within the cell. In other words, an shRNA molecule may be delivered to the cell and cleaved within the cell to form the siRNA that then transiently inhibits the LDTF, or an siRNA molecule itself may be delivered to the target cell. In both scenarios, the inhibition of the LDTF is ultimately accomplished by an siRNA.

An siRNA or shRNA molecule that may be used in accordance with the embodiments described herein may be a single stranded siRNA molecule that includes an siRNA sequence (antisense strand), or a double stranded siRNA molecule that includes an siRNA sequence (antisense strand) and a complementary passenger strand (sense strand). An shRNA molecule that may be used in accordance with the embodiments described herein may include an siRNA sequence (antisense strand), a complementary passenger strand (sense strand), and an intervening loop sequence that joins the siRNA and passenger strands. Exemplary siRNA sequences, complementary passenger strands, and loop sequences that may be used to form the siRNA or shRNA molecules used with the embodiments described herein include, but are not limited to, the sequences described below in Tables 1 and 2.

TABLE 1
Sequences that form siRNA and shRNA molecules for transiently
inhibiting SNAI2. Each row corresponds to sequences that form
an siRNA (Column 1 and Column 2) or shRNA (Column 1, Column 2,
and Column 3):

| | Column 1: | Column 2: | Column 3: |
|---|---|---|---|
| Species | siRNA Sequence | Passenger strand sequence | Loop Seq. |
| Rat | AACGAUUUCCUAGCCUGGGCAU | GCCCAGGCUAGGAAAUCGUUNN | ACGAUUU |
| Rat | uUAAUGGGACUUUCUGAACCAC | GGUUCAGAAAGUCCCAUUAGNN | UAAUGGG |
| Rat | UGCUUAUGUUUGGCCAGACCAG | GGUCUGGCCAAACAUAAGuANN | GCUUAUG |
| Rat | AGAUCUUGCAGACACAAGGCAA | GCCUUGUGUCUGCAAGAUuUNN | GAUCUUG |
| Rat | AGCUCUAUUGCAGUGAGGGCAA | GCCCUCACUGCAAUAGAGuUNN | GCUCUAU |
| Rat | GAGUGGGUCUGCAGAUGUGCCC | GCACAUCUGCAGACCCAuUuNN | AGUGGGU |
| Rat | GGUCUUAUUGCAUAAAUUGCAC | GCAAUUUAUGCAAUAAGAuuNN | GUCUUAU |
| Rat | UGUCCAUACAGUUAUAGGGCUG | GCCCUAUAACUGUAUGGAuANN | GUCCAUA |
| Rat | uAGACUCCUCAUGUUUAUGCAG | GCAUAAACAUGAGGAGUuUGNN | AGACUCC |
| Rat | uAGUGUGCCACACAGCAGCCAG | GGCUGCUGUGUGGCACAuUGNN | AGUGUGC |
| Rat | GUUAUAGGGCUGUACGCUCCUG | GGAGCGUACAGCCCUAUAAuNN | UUAUAGG |
| Rat | GUGGGUCUGCAGAUGUGCCCUC | GGGCACAUCUGCAGACCuAuNN | UGGGUCU |
| Rat | AGUAGAAUAGGUCUUAUUGCAU | GCAAUAAGACCUAUUCUAuUNN | GUAGAAU |
| Rat | UUCUAAUGUGUCCUUGAAGCAG | GCUUCAAGGACACAUUAGAANN | UCUAAUG |
| Rat | AGCCUUGCCACAGAUCUUGCAG | GCAAGAUCUGUGGCAAGGuUNN | GCCUUGC |
| Rat | GGGGGUCUGAAAGCUUGGGCUG | GCCCAAGCUUUCAGACCuuuNN | GGGGUCU |
| Rat | UUACAUCAGAGUGGGUCUGCAG | GCAGACCCACUCUGAUGUAANN | UACAUCA |
| Rat | AGUAAGAGGAGAAAGGCCGCUG | GCGGCCUUUCUCCUCUUAuUNN | GUAAGAG |
| Rat | GGGCAUCACAGUGCAGCUGCUU | GCAGCUGCACUGUGAUGuuuNN | GGCAUCA |
| Rat | AGCUGAACGAUUUCCUAGCCUG | GGCUAGGAAAUCGUUCAGuUNN | GCUGAAC |
| Rat | UGUGCAUCUUCAGGGCACCCAG | GGGUGCCCUGAAGAUGCAuANN | GUGCAUC |
| Rat | AGUGGGUCUGCAGAUGUGCCCU | GGCACAUCUGCAGACCCAuUNN | GUGGGUC |
| Rat | UUUGCAGCUGAACGAUUUCCUA | GGAAAUCGUUCAGCUGCAAANN | UUGCAGC |
| Rat | UUGCACUGAAACUUCUCAGCUU | GCUGAGAAGUUUCAGUGuAANN | UGCACUG |
| Rat | GGGUCUGGAGAAAGCCUUGCCA | GCAAGGCUUUCUCCAGAuuuNN | GGUCUGG |
| Human | GAUUGCGUCACUCAGUGUGCUA | GCACACUGAGUGACGCAAUuNN | AUUGCGU |
| Human | UUCGAGUAAACAUUGAUUGCGU | GCAAUCAAUGUUUACUCGAANN | UCGAGUA |
| Human | GACACGGCGGUCCCUACAGCAU | GCUGUAGGGACCGCCGUGUuNN | ACACGGC |
| Human | UGGGCGUGGAAUGGAGCAGCGG | GCUGCUCCAUUCCACGCuuANN | GGGCGUG |
| Human | UUUGACCUGUCUGCAAAUGCUC | GCAUUUGCAGACAGGUCAAANN | UUGACCU |
| Human | GGGCAUCGCAGUGCAGCUGCUU | GCAGCUGCACUGCGAUGuuuNN | GGCAUCG |
| Human | AGGUUGUUUAUAGACAUAGCUA | GCUAUGUCUAUAAACAAuuUNN | GGUUGUU |
| Human | AAUGGGUCUGCAGAUGAGCCCU | GGCUCAUCUGCAGACCCAUUNN | AUGGGUC |
| Human | AGCUAAUCUUUCCCUCCUCCCC | GGAGGAGGGAAAGAUUAGuUNN | GCUAAUC |
| Human | GGUCUUAUUGCAUAAAUUGCAC | GCAAUUUAUGCAAUAAGAuuNN | GUCUUAU |
| Human | UGCUUAUGUUUGGCCAGCCCAG | GGGCUGGCCAAACAUAAGuANN | GCUUAUG |
| Human | UUAGUUCAACAAUGGCAACCAG | GGUUGCCAUUGUUGAACUAANN | UAGUUCA |
| Human | uUGGUUGUGGUAUGACAGGCAU | GCCUGUCAUACCACAACuAGNN | UGGUUGU |
| Human | uUAAUGGGGCUUUCUGAGCCAC | GGCUCAGAAAGCCCCAUUAGNN | UAAUGGG |
| Human | AGUAAGACAAUGGAGCAUGCGC | GCAUGCUCCAUUGUCUUAuUNN | GUAAGAC |
| Human | AAGGGGUAUUGUAUUAUUGCAU | GCAAUAAUACAAUACCCuUUNN | AGGGGUA |
| Human | AAGGAGCGCGGCAUCUUGCCAG | GGCAAGAUGCCGCGCUCuUUNN | AGGAGCG |
| Human | UUUGGUACUAAUCAUGAAGCAA | GCUUCAUGAUUAGUACCAAANN | UUGGUAC |
| Human | AGGCCAUUGGGUAGCUGGGCGU | GCCCAGCUACCCAAUGGuuUNN | GGCCAUU |
| Human | AUGGGUCUGCAGAUGAGCCCUC | GGGCUCAUCUGCAGACCuAUNN | UGGGUCU |
| Human | AGUUGAAUAGGUCUUAUUGCAU | GCAAUAAGACCUAUUCAAuUNN | GUUGAAU |
| Human | AGUCCACACAGUGAUGGGGCUG | GCCCCAUCACUGUGUGGAuUNN | GUCCACA |
| Human | uAGAUUCCUCAUGUUUGUGCAG | GCACAAACAUGAGGAAUuUGNN | AGAUUCC |
| Human | uAGUGUGCUACACAGCAGCCAG | GGCUGCUGUGUAGCACAuUGNN | AGUGUGC |
| Human | GUGAUGGGGCUGUAUGCUCCUG | GGAGCAUACAGCCCCAUuAuNN | UGAUGGG |
| Mouse | GGUUGCGCCACUCAGUGUGCCA | GCACACUGAGUGGCGCAAuuNN | GUUGCGC |
| Mouse | GUUGCGCCACUCAGUGUGCCAC | GGCACACUGAGUGGCGCAAuNN | UUGCGCC |
| Mouse | AACGGUCCAGGCGGCGGUCCUA | GGACCGCCGCCUGGACCGUUNN | ACGGUCC |
| Mouse | AACGAUUUCCUAGACUGGGCAU | GCCCAGUCUAGGAAAUCGUUNN | ACGAUUU |
| Mouse | UAUAGGCGUGGCUAUUAACCGU | GGUUAAUAGCCACGCCUAUANN | AUAGGCG |
| Mouse | GGAGUACUUUAUAAGGCACCUG | GGUGCCUUAUAAAGUACUuuNN | GAGUACU |
| Mouse | AGCCCUAUUGCAGUGAGGGCAA | GCCCUCACUGCAAUAGGGuUNN | GCCCUAU |
| Mouse | AAGGCGGGGGACUUACACGCCC | GCGUGUAAGUCCCCCGCuUUNN | AGGCGGG |
| Mouse | UGGUUGAAUAUAGGCGUGGCUA | GCCACGCCUAUAUUCAAuuANN | GGUUGAA |
| Mouse | GGCAUUGUAUUACUGCAUCCAU | GGAUGCAGUAAUACAAUGuuNN | GCAUUGU |
| Mouse | GGUCUUAUUGCAUAAAUUGCAC | GCAAUUUAUGCAAUAAGAuuNN | GUCUUAU |
| Mouse | UAAUAGGGCUGUAUGCUCCCGA | GGGAGCAUACAGCCCUAUUANN | AAUAGGG |
| Mouse | UGUCCAUACAGUAAUAGGGCUG | GCCCUAUUACUGUAUGGAuANN | GUCCAUA |
| Mouse | UGCUUGUGUUUGGCCAGCCCAG | GGGCUGGCCAAACACAAGuANN | GCUUGUG |
| Mouse | uUAAUGGGACUUUCUGAACCAC | GGUUCAGAAAGUCCCAUUAGNN | UAAUGGG |
| Mouse | UGUGCAUCUUCAGGGCACCCAG | GGGUGCCCUGAAGAUGCAuANN | GUGCAUC |
| Mouse | UAGUUGGGCUUCUUGGAGGCGU | GCCUCCAAGAAGCCCAAuUANN | AGUUGGG |
| Mouse | AAGGAGCGCGGCAUGGUGGCUG | GCCACCAUGCCGCGCUCuUUNN | AGGAGCG |
| Mouse | GGAGAGUGGAGUGGAGCUGCCG | GCAGCUCCACUCCACUCUuuNN | GAGAGUG |
| Mouse | GUAAUAGGGCUGUAUGCUCCCG | GGAGCAUACAGCCCUAUUAuNN | UAAUAGG |
| Mouse | AGAUCUUGCAGACACAAGGCAA | GCCUUGUGUCUGCAAGAUuUNN | GAUCUUG |
| Mouse | uAGACUCCUCAUGUUUAUGCAG | GCAUAAACAUGAGGAGUuUGNN | AGACUCC |
| Mouse | GGGCAUCACAGUGCAGCUGCUU | GCAGCUGCACUGUGAUGuuuNN | GGCAUCA |
| Mouse | GUGGGUCUGCAGAUGUGCCCUC | GGGCACAUCUGCAGACCuAuNN | UGGGUCU |
| Mouse | UUUGAGUAAACACUGGUUGCGC | GCAACCAGUGUUUACUCAAANN | UUGAGUA |

TABLE 2
Sequences that form siRNA and shRNA molecules for transiently
inhibiting PRRX1. Each row corresponds to sequences that form
an siRNA (Column 1 and Column 2) or shRNA (Column 1, Column 2,
and Column 3):

| | Column 1: | Column 2: | Column 3: |
|---|---|---|---|
| Species | siRNA Sequence | Passenger strand sequence | Loop Seq. |
| Rat | UAAUCGGUGGGUCUGGGAGCAG | GCUCCCAGACCCACCGAUUANN | AAUCGGU |
| Rat | uGUUCGCUGAAGCCAAGUCCGG | GGACUUGGCUUCAGCGAAuGNN | GUUCGCU |
| Rat | uUGUACGGAGAGGCUGUCCCCC | GGGACAGCCUCUCCGUAuAGNN | UGUACGG |
| Rat | GUUGCCGCUCCAGAACGUGCCC | GCACGUUCUGGAGCGGCAAuNN | UUGCCGC |
| Rat | UGUACGGAGAGGCUGUCCCCCA | GGGGACAGCCUCUCCGUAuANN | GUACGGA |
| Rat | uUCGAAUCAACACCAAACGCUG | GCGUUUGGUGUUGAUUCGAGNN | UCGAAUC |
| Rat | UUUCGUCCGCCUGUGCCGCCAC | GGCGGCACAGGCGGACGAAANN | UUCGUCC |
| Rat | UUAACGCUCGAAUCAACACCAA | GGUGUUGAUUCGAGCGUUAANN | UAACGCU |
| Rat | GGUGUCGAGGUUGCCAGGGCUA | GCCCUGGCAACCUCGACAuuNN | GUGUCGA |
| Rat | UUGCCGCUCCAGAACGUGCCCG | GGCACGUUCUGGAGCGGuAANN | UGCCGCU |
| Rat | uACGCUCCAAGGCCUGCAGCUG | GCUGCAGGCCUUGGAGCGUGNN | ACGCUCC |
| Rat | GUACGGAGAGGCUGUCCCCCAG | GGGGGACAGCCUCUCCGUAuNN | UACGGAG |
| Rat | uUUUCGUCCGCCUGUGCCGCCA | GCGGCACAGGCGGACGAAAGNN | UUUCGUC |
| Rat | UUCGGUUCGUUCGCUGAAGCCA | GCUUCAGCGAACGAACCGAANN | UCGGUUC |
| Rat | UUCGGUUCUGAAACCACACCUG | GGUGUGGUUUCAGAACCGAANN | UCGGUUC |
| Rat | AUGGCGCUGUACGGAGAGGCUG | GCCUCUCCGUACAGCGCuAUNN | UGGCGCU |
| Rat | UGCCGCUCCAGAACGUGCCCGU | GGGCACGUUCUGGAGCGGuANN | GCCGCUC |
| Rat | AGACACGCUCCAAGGCCUGCAG | GCAGGCCUUGGAGCGUGUuUNN | GACACGC |
| Rat | UGCGCAGGGCUAUUGUUGGCAC | GCCAACAAUAGCCCUGCGuANN | GCGCAGG |
| Rat | UAUUGGCCAGCAUGGCUCGCUC | GCGAGCCAUGCUGGCCAAUANN | AUUGGCC |
| Rat | AGCAUCCGGGUAAUGGGUCCUC | GGACCCAUUACCCGGAUGuUNN | GCAUCCG |
| Rat | UGUUGGCCAUGUUGAUACCCUG | GGGUAUCAACAUGGCCAAuANN | GUUGGCC |
| Rat | uAUGGUCUUCCCUCCAAUCCCA | GGAUUGGAGGGAAGACCAUGNN | AUGGUCU |
| Rat | AGGUUGGCAAUGCUGUUGGCCA | GCCAACAGCAUUGCCAAuuUNN | GGUUGGC |
| Rat | uUGUUGGCCAUGUUGAUACCCU | GGUAUCAACAUGGCCAAuAGNN | UGUUGGC |
| Human | UGUACGGAGACGCUGUCCCCCA | GGGGACAGCGUCUCCGUAuANN | GUACGGA |
| Human | uUGUACGGAGACGCUGUCCCCC | GGGACAGCGUCUCCGUAuAGNN | UGUACGG |
| Human | UAAUCGGUGGGUCUCGGAGCAG | GCUCCGAGACCCACCGAUUANN | AAUCGGU |
| Human | GUACGGAGACGCUGUCCCCCAG | GGGGGACAGCGUCUCCGUAuNN | UACGGAG |
| Human | UUCGGUUCUGAAACCACACCUG | GGUGUGGUUUCAGAACCGAANN | UCGGUUC |
| Human | GUGUCCGCUCAAAGACACGCUC | GCGUGUCUUUGAGCGGAuAuNN | UGUCCGC |
| Human | uACGCUCCAAAGCCUGCAGCUG | GCUGCAGGCUUUGGAGCGUGNN | ACGCUCC |
| Human | uAUCCGCCUGUGCCGCCACCAU | GGUGGCGGCACAGGCGGAUGNN | AUCCGCC |
| Human | GGCACGAAAGAGAUGUGGGCAU | GCCCACAUCUCUUUCGUGuuNN | GCACGAA |
| Human | UGCGUGCUGUUCAGCAAGCCUA | GGCUUGCUGAACAGCACGuANN | GCGUGCU |
| Human | uAGGACGAGGUACGAUGGGCUG | GCCCAUCGUACCUCGUCuUGNN | AGGACGA |
| Human | AUGCGUGCUGUUCAGCAAGCCU | GCUUGCUGAACAGCACGuAUNN | UGCGUGC |
| Human | AGACACGCUCCAAAGCCUGCAG | GCAGGCUUUGGAGCGUGUuUNN | GACACGC |
| Human | GACGAGGUACGAUGGGCUGCUC | GCAGCCCAUCGUACCUCGUuNN | ACGAGGU |
| Human | GGCGGGCAAGGUCUUCUCGCAC | GCGAGAAGACCUUGCCCGuuNN | GCGGGCA |
| Human | GAGGUACGAUGGGCUGCUCCAC | GGAGCAGCCCAUCGUACuUuNN | AGGUACG |
| Human | UAUACCCUAAAUGUCAUUCCCU | GGAAUGACAUUUAGGGUAUANN | AUACCCU |
| Human | AGAAUCCGUUAUGAAGCCCCUC | GGGGCUUCAUAACGGAUUuUNN | GAAUCCG |
| Human | UGGAGUACAUUAAGCAAAGCAU | GCUUUGCUUAAUGUACUuuANN | GGAGUAC |
| Human | UGUAUUGUUUGGGAAAUUGCAA | GCAAUUUCCCAAACAAUAuANN | GUAUUGU |
| Human | AGGUUGUCCUAUUCCUUCGCUG | GCGAAGGAAUAGGACAAuuUNN | GGUUGUC |
| Human | AUAGAGCCUCACAACACCCCUG | GGGGUGUUGUGAGGCUCUAUNN | UAGAGCC |
| Human | AUACCCUAAAUGUCAUUCCCUU | GGGAAUGACAUUUAGGGUAUNN | UACCCUA |
| Human | UGUAGUUGGUCAAGUCCACCUG | GGUGGACUUGACCAACUAuANN | GUAGUUG |
| Human | GGUUAUGUCUUGACAUUUGCAG | GCAAAUGUCAAGACAUAAuuNN | GUUAUGU |
| Mouse | AGCGCGUUCCCCCUCUCUGCAC | GCAGAGAGGGGGAACGCGuUNN | GCGCGUU |
| Mouse | UAAUCGGUUGGUCUGGGAGCAG | GCUCCCAGACCAACCGAUUANN | AAUCGGU |
| Mouse | uUGUACGGAGAGGCUGUCCCCC | GGGACAGCCUCUCCGUAuAGNN | UGUACGG |
| Mouse | GUUGCCGCUCCAGAACGUGCCC | GCACGUUCUGGAGCGGCAAuNN | UUGCCGC |
| Mouse | UGUACGGAGAGGCUGUCCCCCA | GGGGACAGCCUCUCCGUAuANN | GUACGGA |
| Mouse | uAUCCGGGUAAUGUGUCCGCUC | GCGGACACAUUACCCGGAUGNN | AUCCGGG |
| Mouse | GUGUCCGCUCAAAGACACGCUC | GCGUGUCUUUGAGCGGAuAuNN | UGUCCGC |
| Mouse | uUCGAAUCAACACCAAACGCUG | GCGUUUGGUGUUGAUUCGAGNN | UCGAAUC |
| Mouse | UUAACGGUGACAUCAUAACCUA | GGUUAUGAUGUCACCGUUAANN | UAACGGU |
| Mouse | UUGCCGCUCCAGAACGUGCCCG | GGCACGUUCUGGAGCGGuAANN | UGCCGCU |
| Mouse | uUCGGUUACUUCGCUAAAGCCA | GCUUUAGCGAAGUAACCGAGNN | UCGGUUA |
| Mouse | GUACGGAGAGGCUGUCCCCCAG | GGGGGACAGCCUCUCCGUAuNN | UACGGAG |
| Mouse | uUUUCGUCUGCUUGUGCCGCCA | GCGGCACAAGCAGACGAAAGNN | UUUCGUC |
| Mouse | UUUCGUCUGCUUGUGCCGCCAC | GGCGGCACAAGCAGACGAAANN | UUCGUCU |
| Mouse | GACGAGGUACGAUGGGUUGCUC | GCAACCCAUCGUACCUCGUuNN | ACGAGGU |
| Mouse | UUCGGUUCUGAAACCACACCUG | GGUGUGGUUUCAGAACCGAANN | UCGGUUC |
| Mouse | AUGGCGCUGUACGGAGAGGCUG | GCCUCUCCGUACAGCGCuAUNN | UGGCGCU |
| Mouse | UUCGGGACAAGUAACUUUCCCU | GGAAAGUUACUUGUCCCGAANN | UCGGGAC |
| Mouse | AAGCGCCAACAGAAGAAGGCUU | GCCUUCUUCUGUUGGCGuUUNN | AGCGCCA |
| Mouse | UGCCGCUCCAGAACGUGCCCGU | GGGCACGUUCUGGAGCGGuANN | GCCGCUC |
| Mouse | GGUACGUGCUCAGAUAAAGCUG | GCUUUAUCUGAGCACGUAuuNN | GUACGUG |
| Mouse | GGAGCGCAGUAGCUAUAUGCCU | GCAUAUAGCUACUGCGCUuuNN | GAGCGCA |
| Mouse | GAGCGCAGUAGCUAUAUGCCUU | GGCAUAUAGCUACUGCGuUuNN | AGCGCAG |
| Mouse | UUCCCGCUCGAAUCAACACCAA | GGUGUUGAUUCGAGCGGGAANN | UCCCGCU |
| Mouse | GAGCGAGGAAAGGGGAGGCCAG | GGCCUCCCCUUUCCUCGuUuNN | AGCGAGG |

The siRNA and shRNA molecules may be delivered to the target somatic cell via non-viral delivery methods including, but not limited to, liposomal delivery methods, targeted liposomal delivery, nanoparticle delivery methods, or other methods known in the art. Transient inhibition of a single LDTF as described above can successfully affect nuclear reprogramming of lineage committed somatic cells to their pre-lineage commitment state of cellular plasticity. Once a state of cellular plasticity is achieved, the siRNA/shRNA treated cells, when provided appropriate culture conditions in vitro or specific microenvironments in vivo, can be directed to differentiate in the direction of a desired and specific lineage trajectory and form either differentiated cells of alternative lineage to that of the starting cell (i.e., transdifferentiated somatic cells), or dedifferentiated stem cells of multipotent or pluripotent differentiation capacity.

Thus, the method of producing a transdifferentiated somatic cell or a dedifferentiated stem cell according to the embodiments described herein also includes a step of exposing the reprogrammed lineage committed somatic cell to an environment specific to a desired transdifferentiated somatic cell (i.e., the desired cell lineage or cell type) or a desired stem cell (e.g., pluripotent or multipotent stem cell). For example, reprogrammed somatic cells (i.e., somatic cells treated with an siRNA or shRNA may be incubated in in vitro culture conditions specific to the transdifferentiated somatic cell.

In accordance with the embodiments described herein, a lineage committed somatic cell may be reprogrammed to become a cell of any desired lineage or any type of terminally differentiated cell. In one embodiment, the starting cell is a lineage committed fibroblast cell. Examples of transdifferentiated somatic cells that can be produced by the methods provided herein include, but are not limited to, cells of adipocytic lineage including, but not limited to, adipocytes; cells of osteogenic lineage including, but not limited to, osteocytes; cells of chondrogenic lineage including, but not limited to, chondrocytes; cells of cardiovascular lineage including, but not limited to, cardiac myocytes, cells of pancreatic lineage including, but not limited to, pancreatic beta cells; cells of ophthalmic lineage including, but not limited to, retinal pigment epithelial cells; cells of hepatic lineage including, but not limited to, hepatocytes; cells of pulmonary lineage including, but not limited to, pneumocytes; cells of renal lineage including, but not limited to, nephron cells. In other embodiments, the lineage committed somatic cell may be reprogrammed to become a desired type of dedifferentiated stem cell. Examples of dedifferentiated stem cells that can be produced by the methods provided herein include but are not limited to, dedifferentiated pluripotent stem cells, and dedifferentiated multipotent stem cells including, but not limited to, mesenchymal stem cells.

Table 3 below shows examples of reprogrammed somatic cells and a corresponding in vitro environment to which it may be exposed to produce each desired transdifferentiated or dedifferentiated cell type or lineage.

TABLE 3

| | | Transdifferentiated or |
|---|---|---|
| Starting Somatic Cell | Environment(**) | Dedifferentiated Cell |
| lineage committed | cell culture media specific | cell of adipocytic |
| fibroblast treated with | to a cell of adipocytic | lineage |
| siRNA or shRNA | lineage | |
| lineage committed | cell culture media specific | adipocyte |
| fibroblast treated with | to an adipocyte | |
| siRNA or shRNA | | |
| lineage committed | cell culture media specific | cell of osteogenic |
| fibroblast treated with | to a cell of osteogenic | lineage |
| siRNA or shRNA | lineage | |
| lineage committed | cell culture media specific | osteocyte |
| fibroblast treated with | to an osteocyte | |
| siRNA or shRNA | | |
| lineage committed | cell culture media specific | cell of chondrogenic |
| fibroblast treated with | to a cell of chondrogenic | lineage |
| siRNA or shRNA | lineage | |
| lineage committed | cell culture media specific | chondrocyte |
| fibroblast treated with | to a chondrocyte | |
| siRNA or shRNA | | |
| lineage committed | cell culture media specific | cell of cardiovascular |
| fibroblast treated with | to a cell of cardiovascular | lineage |
| siRNA or shRNA | lineage | |
| lineage committed | cell culture media specific | cardiac myocyte |
| fibroblast treated with | to a cardiac myocyte | |
| siRNA or shRNA | | |
| lineage committed | cell culture media specific | cell of pancreatic |
| fibroblast treated with | to a cell of pancreatic | lineage |
| siRNA or shRNA | lineage | |
| lineage committed | cell culture media specific | pancreatic beta cell |
| fibroblast treated with | to a pancreatic beta cell | |
| siRNA or shRNA | | |
| lineage committed | cell culture media specific | cell of ophthalmic |
| fibroblast treated with | to a cell of ophthalmic | lineage |
| siRNA or shRNA | lineage | |
| lineage committed | cell culture media specific | retinal pigment |
| fibroblast treated with | to a retinal pigment | epithelial |
| siRNA or shRNA | epithelial cell | cell |
| lineage committed | cell culture media specific | cell of hepatic |
| fibroblast treated with | to a cell of hepatic lineage | lineage |
| siRNA or shRNA | | |
| lineage committed | cell culture media specific | hepatocyte |
| fibroblast treated with | to a liver cell | |
| siRNA or shRNA | | |
| lineage committed | cell culture media specific | cell of pulmonary |
| fibroblast treated with | to a cell of pulmonary | lineage |
| siRNA or shRNA | lineage | |
| lineage committed | cell culture media specific | pneumocyte |
| fibroblast treated with | to a pulmonary cell | |
| siRNA or shRNA | | |
| Lineage committed | cell culture media specific | Cell of renal lineage |
| fibroblast treated with | to a cell of renal lineage | |
| siRNA or shRNA | | |
| Lineage committed | Cell culture media specific | Nephron cell |
| fibroblast treated with | to a nephron cell | |
| siRNA or shRNA | | |
| lineage committed | mesenchymal stem cell | dedifferentiated |
| fibroblast treated with | media, | multipotent |
| siRNA or shRNA | | stem cells (e.g., |
| | | mesenchymal |
| | | stem cell) |
| lineage committed | embryonic stem cell media | dedifferentiated |
| fibroblast treated with | | pluripotent |
| siRNA or shRNA | | stem cell |

(**) Any appropriate culture media for a desired transdifferentiated or dedifferentiated cell may be used as an environment for inducing transdifferentiation or dedifferentiation of an appropriate siRNA/shRNA treated cell. Media specific to the target transdifferentiated or dedifferentiated cell may be obtained from commercially available sources (e.g., Gibco, PromoCell, HyClone™ and other sources known in the art), or may be generated using suitable ingredients known in the art to create conditions for growing the desired transdifferentiated or dedifferentiated cell.

The methods described herein are advantageous over other current techniques for several reasons. First, the methods of producing dedifferentiated or transdifferentiated cells described herein are performed in the absence of viral or non-viral delivery of exogenous TFs and/or small molecule modulators. Instead, the methods rely only on transient repression of the targeted LDTF using siRNA or shRNA molecules described herein, according to some embodiments. By lifting molecular roadblocks to reprogramming, the described method resets a cell to an earlier point in its developmental timeline, likely derepressing plasticity maintenance factors, enabling the cell's innate differentiation mechanism to then proceed as guided by components provided in its growth medium.

In addition, the methods of producing dedifferentiated or transdifferentiated cells described herein also significantly improve reprogramming efficiency over that of current methods that use miRNA or small molecule modulators to reprogram cells. As previously discussed, current methods result in a reprogramming efficiency that is less than 1%, even though 100% of the starting cell population is exposed to the treatments. Such a low efficiency implies that despite overexpression of transcription factors or exposure to a plethora of pathway modulating small molecules, a significant fraction of the cells resists reprogramming. This is likely due to an unsurmountable barrier to reprogramming often due to retention of the starting cell's Transcriptional Regulatory Network (TRN) and epigenetic state. Thus, non-specific pathway inhibition is not in itself adequate to overcome the epigenetic memory of the starting cell. To make the somatic reprogramming cascade more efficient, the impact of TRN and residual epigenetic state of the starting cell needs to be nullified first through use of the transient suppression described herein. By repressing pivotal lineage instructive TFs of the parental cell, the lineage committed cell gradually erases the transcriptional and epigenetic landscapes tied to lineage commitment, hence the cell reprogramming cascade is met with less hindrance from the residual transcriptional and epigenetic memory of the parent cell thus making the reprogramming process more efficient. Consequently, through the use of siRNA or shRNA molecules to transiently repress one or more key LDTFs, the reprogramming efficiency of lineage committed somatic cells converted to a desired transdifferentiated or dedifferentiated cell type may be more than 1%. The efficiency may vary based on the type of target transdifferentiated or dedifferentiated cell desired. Thus, in certain embodiments, the reprogramming efficiency may be 2% or higher, 3% or higher, 4% or higher, 5% or higher, 6% or higher, 7% or higher, 8% or higher, 9% or higher, 10% or higher, 15% or higher, 20% or higher, 30% or higher, 40% or higher, 50% or higher, 60% or higher, 70% or higher, 80% or higher, 90% or higher, 95% or higher, 96% or higher, 97% or higher, 98% or higher, 99% or higher, or approximately 100%. In other embodiments, the reprogramming efficiency is between 10% and 20%, between 20% and 30%, between 30% and 40%, between 40% and 50%, between 50% and 60%, between 60% and 70%, between 70% and 80%, between 80% and 90%, or between 90% and 100%.

Cells and Populations of Cells

Cells and populations of cells produced using the methods for generating or producing transdifferentiated or dedifferentiated cells discussed above are provided herein.

In certain embodiments, a reprogrammed somatic cell or population of cells is provided. In one embodiment, the reprogrammed somatic cell is a reprogrammed somatic fibroblast that has been treated with an siRNA or shRNA molecule that transiently inhibits expression of an LDTF.

In certain aspects of such embodiment, the LDTF that is transiently inhibited by the siRNA or shRNA molecule is (i) SNAI2, (ii) PRRX1, (iii) HAND1, (iv) CDX2, (v) SNAI2 and PRRX1, (vi) SNAI2 and HAND1, (vii) SNAI2 and CDX2, or (viii) SNAI2, HAND1, and CDX2. In certain aspects, the LDTF that is transiently inhibited by the siRNA or shRNA molecule is SNAI2 alone, PRRX1 alone, HAND1 alone, or CDX2 alone (i.e., transient inhibition of a single LDTF). Through transient suppression of an LDTF and incubation with target media of the desired target cell, reprogrammed somatic cells are, in some embodiments, capable of transdifferentiating to a somatic cell of alternate lineage when placed in an appropriate environment in the absence of viral or non-viral delivery of exogenous TFs and/or small molecule modulators (chemical reprogramming). In other embodiments, the siRNA/shRNA treated somatic cells are capable of dedifferentiating to a multipotent or pluripotent stem cell in the absence of viral or non-viral delivery of exogenous TFs and/or modulators.

In other embodiments, a transdifferentiated cell or population of transdifferentiated somatic cells is provided. Types of transdifferentiated somatic cells that may be provided are discussed above. In one embodiment, a population of transdifferentiated somatic cells include a population of reprogrammed somatic cells (e.g., reprogrammed fibroblasts), such as those described above, that lack permanent genetic modification due to the use of an siRNA or shRNA to transiently—not permanently—suppress an LDTF. Further, transient suppression of the LDTF without the need to introduce any exogenous TFs and/or small molecule modulators (chemical reprogramming) significantly increases the efficiency of reprogramming and the conversion from lineage committed somatic cells (e.g., fibroblasts) to the desired transdifferentiated cells, overcoming an important barrier that currently thwarts clinical application of iPSCs. In certain embodiments, the population of reprogrammed somatic cells (e.g., reprogrammed fibroblasts) were converted to the population of transdifferentiated somatic cells from a population of lineage committed somatic cells (e.g., fibroblast cells) at an efficiency of more than 1%. In other embodiments, the reprogramming efficiency may be 2% or higher, 3% or higher, 4% or higher, 5% or higher, 6% or higher, 7% or higher, 8% or higher, 9% or higher, 10% or higher, 15% or higher, 20% or higher, 30% or higher, 40% or higher, 50% or higher, 60% or higher, 70% or higher, 80% or higher, 90% or higher, 95% or higher, 96% or higher, 97% or higher, 98% or higher, 99% or higher, or approximately 100%.

In other embodiments, a dedifferentiated stem cell or population of dedifferentiated stem cells is provided. Types of dedifferentiated stem cells that may be provided include pluripotent stem cells and multipotent stem cells as discussed above. In one embodiment, a population of dedifferentiated stem cells include a population of reprogrammed somatic cells (e.g., reprogrammed fibroblasts), such as those described above, (without the need for permanent genetic manipulation) due to the use of an siRNA or shRNA to transiently—not permanently—suppress an LDTF. Further, transient suppression of the LDTF without the need to introduce any exogenous TFs and/or small molecule modulators (chemical reprogramming) significantly increases the efficiency of reprogramming and the conversion from lineage committed somatic cells (e.g., lineage committed fibroblasts) to the desired dedifferentiated cells, overcoming an important barrier that currently thwarts clinical application of iPSCs. In certain embodiments, the population of reprogrammed somatic cells (e.g., reprogrammed fibroblasts) were converted to the population of dedifferentiated stem cells from a population of lineage committed somatic cells (e.g., lineage committed fibroblast cells) at an efficiency of more than 1%. The efficiency may vary based on the type of target transdifferentiated or dedifferentiated cell desired. Thus, in other embodiments, the reprogramming efficiency may be 2% or higher, 3% or higher, 4% or higher, 5% or higher, 6% or higher, 7% or higher, 8% or higher, 9% or higher, 10% or higher, 15% or higher, 20% or higher, 30% or higher, 40% or higher, 50% or higher, 60% or higher, 70% or higher, 80% or higher, 90% or higher, 95% or higher, 96% or higher, 97% or higher, 98% or higher, 99% or higher, or approximately 100%.

Cell Culture Systems

To produce the cells or population of cells using the methods described herein, a cell culture system is used in accordance with certain embodiments. A cell culture system includes the materials needed to produce the desired transdifferentiated or dedifferentiated cells.

In one embodiment, a cell culture system to produce a population of transdifferentiated somatic cells is provided. Types of transdifferentiated somatic cells that may be produced are discussed above. The cell culture system for producing transdifferentiated somatic cells includes, but is not limited to, a population of lineage committed somatic cells (e.g., fibroblast cells), an siRNA or shRNA molecule that inhibits expression of an LDTF (as discussed above), and a culture medium that includes a cell culture media specific to the population of transdifferentiated somatic cells, examples of which are shown in Table 3 above.

In another embodiment, a cell culture system to produce a population of dedifferentiated stem cells is provided. Types of dedifferentiated stem cells that may be provided include pluripotent stem cells and multipotent stem cells as discussed above. The cell culture system for producing dedifferentiated stem cells includes, but is not limited to, a population of lineage committed somatic cells (e.g., fibroblast cells), an siRNA or shRNA molecule that inhibits expression of an LDTF (as discussed above), and a culture medium that includes a cell culture media specific to the population of dedifferentiated stem cells, examples of which are shown in Table 3 above.

Methods of Use

The transdifferentiated or dedifferentiated cells produced by the methods above may be used in many medical applications and for treating different conditions. In certain embodiments, a population of reprogrammed somatic cells (i.e., somatic cells treated with an siRNA or shRNA and incubated with the media of desired target cells, as described above) may be used to treat a condition. In other embodiments, a population of transdifferentiated somatic cells may be used to treat a condition. In other embodiments, a population of dedifferentiated stem cells may be used to treat a condition. Non-limiting examples of conditions that may be treated using the reprogrammed somatic cells, transdifferentiated somatic cells, or dedifferentiated stem cells include, but are not limited to, acute or chronic wounds sustained by trauma or as a result of a chronic state (e.g., bed sores) or condition (e.g., wounds resulting from late stage diabetes); osteoarthritis; osteoporosis; loss of bone structure due to trauma or surgical removal, soft tissue damage or loss due to trauma or surgical removal; macular degeneration; diabetes; fibrosis (e.g., lung, kidney, or liver fibrosis); myocardial infarction; chronic or acute heart failure; hepatic failure, pulmonary failure and renal failure.

According to some embodiments, methods for treating the condition may include transplanting or grafting a population of autologous transdifferentiated somatic cells (such as those described above) onto or in a tissue or organ of a subject. “Treating” or “treatment” of a condition may refer to preventing the condition, lessening the severity of the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. According to the embodiments described herein, treatment of the condition may involve engraftment or transplantation of a population of cells. An appropriate grafting or transplantation method may be selected depending on where (i.e., what type of tissue or organ) the population of cells are to be transplanted or grafted. For example, an implantable or injectable graft may be used to treat the condition.

An implantable graft may include a solid matrix that allows cells to be seeded with necessary growth factors (i.e., the environment specific to the population of cells to be implanted), cultured, and then implanted into the subject in the tissue or organ subject to the condition. An injectable graft, which can fill any deficit shape or space in a damaged organ or tissue. The injectable graft involved injection of the dedifferentiated stem cells or reprogrammed somatic cells in a cell suspension containing biomaterials that solidifies in situ by virtue of various crosslinking methods known in the art. The mixture may be injected directly into a tissue or organ or may be exposed or glued to the surface of the tissue or organ.

Non-limiting examples of biomaterials that can be used in injectable grafts include, but are not limited to, inorganic, natural materials like chitosan, alginate, hylauronic acid, fibrin, gelatin, as well as many synthetic polymers. Such materials are often solidified through methods including thermal gelation, photo cross-linking, or chemical cross-linking. The cell suspension may also be supplemented with soluble signals or specific matrix components. Since these grafts can be relatively easily injected into a target area, there is no (or minimal) need for invasive surgery, which reduces cost, patient discomfort, risk of infection, and scar formation. Chemically modified HA may also be used for injectable material for tissue engineering due to its long-lasting effect while maintaining biocompatibility. Cross-linking methods also maintain the material biocompatibility, and its presence in extensive areas of regenerative or stem/progenitor niches make it an attractive injectable material.

In certain embodiments, a population of dedifferentiated stem cells or reprogrammed somatic cells are delivered to a tissue or an organ for the purpose of inducing differentiation of the stem cells or reprogrammed somatic cells into a native cell that is normally present in the tissue or organ based on the environment present in the target tissue or organ in vivo, the population of dedifferentiated stem cells or reprogrammed somatic cells may be delivered via an injectable graft. An injectable graft can be delivered by injection of a cell suspension containing the dedifferentiated stem cells or reprogrammed somatic cells to the target tissue or organ.

In certain embodiments, the transdifferentiated or dedifferentiated cells may be used in a medical procedure to regenerate tissue in wounds or in degenerative diseases. For example, in one embodiment, dedifferentiated cells may be used in wound healing. In that embodiment, native wound bed fibroblasts obtained from a subject having a wound may be subjected to the method of producing dedifferentiated stem cells described above to generate a population of autologous dedifferentiated mesenchymal stem cells that may be applied topically to the subject's wound. When exposed to the in vivo microenvironment of the wound, the autologous mesenchymal stem cells would affect accelerated tissue regeneration, tissue remodeling and wound healing without the need for cell transplantation. This technology provides advantages of easier implementation, greater cost effectiveness, and lower barrier to entry, even in resource challenged settings, e.g., battle zone and frontline hospitals.

In other embodiments, a population of transdifferentiated somatic cells can be used in a graft or transplantation procedure for the treatment of degenerative diseases such as osteoarthritis or osteoporosis. For example, in one embodiment, fibroblasts obtained from a subject suffering from osteoarthritis may be subjected to the method of transdifferentiation described above to generate a population of autologous chondrocytes (i.e., cartilage cells) that may be grafted or transplanted into or onto the subject's joints affected by osteoarthritis. In another embodiment, fibroblasts obtained from a subject suffering from osteoporosis may be subjected to the method of transdifferentiation described above to generate a population of autologous osteocytes (i.e., bone cells) that may be grafted or transplanted into or onto the subject's bones affected by osteoporosis.

In other embodiments, a population of transdifferentiated somatic cells can be used in a graft or transplantation procedure for a reconstructive procedure. For example, in one embodiment, fibroblasts obtained from a subject in need of an orthopedic or oral maxillofacial reconstructive procedure due to trauma or other loss of bone structure may be subjected to the method of transdifferentiation described above to generate a population of autologous osteocytes (i.e., bone cells) that may be used to reconstruct the subject's bone structure. In another embodiment, fibroblasts obtained from a subject in need of or that has elected a reconstructive plastic surgery procedure may be subjected to the method of transdifferentiation described above to generate a population of autologous adipocytes (i.e., fat cells) that may be used in reconstructive plastic surgery procedures.

In other embodiments, the methods described herein may be employed in vivo to effect transdifferentiation of scar tissue in diseased tissues. For example, in one embodiment, a therapeutic siRNA or shRNA may be administered to a subject with scar tissue in the lung resulting from pulmonary fibrosis, which is caused by a process involving fibroblast proliferation. Any suitable delivery method may be used including, but not limited to, injection, i.v. infusion, and inhalation. The siRNA or shRNA may be designed to suppress an LDTF such as SNAI2 PRRX1, HAND1, or CDX2 (or a combination thereof), and when delivery of the siRNA is targeted to the fibrotic cells in the lung, the siRNA or shRNA transiently inhibits the LDTF and reprograms the fibrotic cells to lung cells due to exposure to in vivo microenvironmental conditions specific to the lung, thereby reversing the pathologic effects of the fibrosis. In another embodiment, a therapeutic siRNA or shRNA or shRNA may be administered to a subject with scar tissue in the liver due to liver cirrhosis. The siRNA or shRNA may be designed to suppress an LDTF such as SNAI2 PRRX1, HAND1, or CDX2 (or a combination thereof), and when delivery of the siRNA or shRNA is targeted to the fibrotic cells in the liver, the siRNA or shRNA transiently inhibits the LDTF and reprograms the fibrotic cells to liver cells due to exposure to in vivo microenvironmental conditions specific to the liver, thereby reversing the pathologic effects of the fibrosis.

In other embodiments, the methods described herein may be employed in vivo to effect transdifferentiation of tissues in subjects suffering from a chronic disease. For example, in one embodiment, a therapeutic siRNA or shRNA may be administered to a subject with diabetes mellitus. The siRNA or shRNA may be designed to suppress an LDTF, and when delivery of the siRNA or shRNA is targeted to the islet of the pancreas, the siRNA or shRNA transiently inhibits the LDTF and triggers the pancreatic cells to regenerate native normal pancreatic beta cells. In another embodiment, a therapeutic siRNA or shRNA may be administered to a subject with heart failure. The siRNA or shRNA may be designed to suppress an LDTF, and when delivery of the siRNA or shRNA is targeted to the myocardium, the siRNA or shRNA transiently inhibits the LDTF and triggers the myocardial cells to regenerate native normal myocardial cells. therapeutic siRNA or shRNA may be administered to a subject with diabetes mellitus. In another embodiment, a therapeutic siRNA or shRNA may be administered to a subject with macular degeneration. The siRNA or shRNA may be designed to suppress an LDTF, and when delivery of the siRNA or shRNA is targeted to the retina, the siRNA or shRNA transiently inhibits the LDTF and triggers the retinal cells to regenerate native normal retinal pigment epithelial cells.

In other embodiments, dedifferentiated cells may be used in regeneration of a tissue or organ to be transplanted into a patient in need of a transplant. In such an embodiment, native fibroblasts obtained from a subject in need of a tissue or organ transplant may be subjected to the method of dedifferentiation described above to generate a population of autologous dedifferentiated pluripotent stem cells. Such Pluripotent stem cells may be utilized to generate cells or tissues of any germ layer origin for utilization through cell and/or tissue transplantation, to replace diseased cells or organs. Risk of tissue or organ rejection will be nullified as the cells/tissues/organs will be autologous in origin.

### WORKING EXAMPLES

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1: Generation of Transdifferentiated and Dedifferentiated Somatic Cells from Lineage Committed Fibroblast Cells

Using the intra-species somatic cell hybrid model described above, SNAI2 and PRRX1 were identified as pivotal TFs that preserve mesenchymal fate in rat embryonic fibroblasts (REFs) and demonstrate that siRNA-mediated transient knockdown of individual factors results in direct conversion of REFs into functional adipocytes, chondrocytes or osteocytes. Additionally, the studies described below show that siRNA-mediated transient knockdown of SNAI2 alone is sufficient to transform REFs to a dedifferentiated pluripotent stem cell (dPSC) state that forms embryoid bodies and is capable of triple germ layer differentiation in the absence of exogenous TFs and/or small molecule modulators (chemical reprogramming). These results establish for the first time that transient repression of a single lineage defining TF of a somatic cell can effectively overcome molecular safeguards preserving cell identity and is sufficient to induce its transdifferentiation to alternative cell fates or its dedifferentiation to primitive cell states.

Materials and Methods

Cell lines and Culture Conditions. The rat hepatoma cell line FTO2B is an ouabain-resistant, thymidine kinase TK-deficient H4IIEC3 derived clone (Killary & Fournier 1984). RAT-1 is a SV40-transformed rat embryo fibroblast (REF) cell line that expresses functional thymidine kinase (Botchan et al. 1976). HF is a hybrid cell line generated by the fusion of FTO2B and RAT-2 cells (Bulla et al. 2012). All cell lines were maintained in 1:1 Ham's F12/Dulbecco's modified eagle's medium (DMEM) containing 10% fetal bovine serum (FBS) (GIBCO BRL) and 5 μg/100 ml penicillin-streptomycin (GIBCO) at 37° C. in a humid, water-jacketed 5% CO₂ incubator.

Generation of Hepatoma X Fibroblast Hybrids. FTO2B cells were plated with RAT-1 cells (10⁶ of each cell type). After 24 hours, the mixed cell monolayers were fused to create the FR(2) hybrid cell line using 50% polyethylene glycol (50% w/v) for 1 minute. The following day, cells were split 1:20 into medium containing 3 mM Ouabain and Hypoxanthine-Aminopterin-Thymidine (HAT) medium to select against parental fibroblast and hepatoma cells, respectively. After 2-3 weeks no parental cells survived the selection strategy, but numerous surviving cells were observed, pooled (>100 clones/pool) and expanded in Hypoxanthine-Aminopterin-Thymidine (HAT) medium, that were ostensibly hybrid cells and had survived because of their being both ouabain-resistant as well as having functionally active thymidine kinase enzyme. All cell lines were maintained in 1:1 Ham's F12/Dulbecco's modified eagle's medium (DMEM) containing 5% fetal bovine serum (FBS) (GIBCO BRL) and 5 μg/100 ml penicillin-streptomycin (GIBCO) at 37° C. in a humid water-jacketed 5% CO₂ incubator. 70% confluence in the monolayer of cells was achieved. Chromosome spreads and RNA isolation were carried out for each cell line.

Chromosome Spreads. To analyze cell hybrids for complete karyotypes, sub-confluent cell monolayers were exposed to 0.05 μM colcemid (a mitosis inhibitor) for 40 min and cells harvested. Cells were incubated in 0.075 M KCl for 10 min and then treated with methanol/acetic acid (3:1). Cells were dropped onto cold, wet slides then the slides stained with crystal violet and scored for chromosome numbers per spread for 10-15 spreads. To confirm that no parental cells had survived the selection strategy, surviving cells in HAT medium were pooled and tested to find out whether these presumed FR(2) hybrid cells were indeed true hybrids by determining the number of chromosomes present in the cells. Metaphase chromosome spreads of each parental cell line and the somatic cell hybrids were prepared and the chromosomes were quantitated.

Microarray Analysis. RNA was extracted using RNA Easy Mini Kit (Qiagen) according to manufacturer's protocol. RNA purity and concentration were determined using an Epoch Nano-drop spectrophotometer at 260 and 280 nm. Following RNA extraction, the RNA samples from all cell lines were sent for Microarray Analysis. RNA was hybridized to Affymetrix GeneChip® Rat Genome 230 2.0 Expression Arrays (Affymetrix, Inc., Santa Clara, Calif.) utilizing the GeneChip (Expression 3′ Amplification One-Cycle Target Labeling and Control Reagents kit according to the manufacturer's instructions). The chips were scanned with a GeneChip Scanner model 3000 7G Plus. Gene expression was detected and normalized.

cDNA synthesis and qRT-PCR Analysis. RNA was extracted from 70-80%-confluent monolayers using RNA Easy Mini Kit (Qiagen) according to manufacturer's protocol. RNA purity and concentration were determined using an Epoch Nano-drop spectrophotometer at 260 and 280 nm. Reverse transcription was performed with the Superscript III First Strand cDNA Synthesis kit (Invitrogen) following manufacturers protocol using the Thermal Cycler (Life Technologies, USA). The resulting cDNA was used for qRT-PCR. Quantitative real-time RT-PCR (qRT-PCR) was performed in 20 μl reaction mixture [2 μl of cDNA (5 ng/μl), 6.75 μl of sterile nuclease free water, 10 μl of Fast SYBR® Green Master Mix (Life Technologies, USA) and 1.25 μl of gene specific primer (0.5 μm from IDTDNA)] on a Step One Plus Real-Time PCR System (Life Technologies, USA). PCR parameters consisted of heating at 95° C. for 10s, followed by 40 cycles at 95° C. for 5s and at 60° C. for 30s. The amplification signal of the target genes' mRNA was normalized against endogenous house-keeping genes' mRNA in the same reaction. The list of primers used for q-RT-PCR are as follows:

TABLE 4

| PRIMER | PRIMER SEQUENCE |
|---|---|
| PRRX1 | F-5′gaaccgaagctgggagaaa-3′ |
| | R-5′aggagagagagagagagaga-3′ |
| SNAI2 | F-5′gcagacccactctgatgtaaag-3′ |
| | R-5′cagccagactcctcatctttat-3′ |
| Col1a1 | F-5′aggtgctatttaacaagggagaa-3′ |
| | R-5′agataggcccaggaactagaa-3′ |
| Cepba | F-5′-gagggactggagttatgacaag-3′ |
| | R-5′tgggacacagagaccagata-3′ |
| Ppia | F-5′gtggtctttgggaaggtgaa-3′ |
| | R-5′gtcggagatggtgatcttcttg-3′ |
| Runx2 | F-5′gaagaaccaagtggcgagat-3′ |
| | R-5′gtaaagacggtgatggtcagag-3′ |
| Spp1 | F-5′cagccaaggaccaactacaa-3′ |
| | R-5′tgccaaactcagccactt-3′ |
| Sox9 | F-5′catcaagacggagcaactga-3′ |
| | R-5′tgtagtgcggaaggttgaag-3′ |
| Acan | F-5′ctggaagagaggacacagaaac-3′ |
| | R-5′ctcctgaagctgaggtctcta-3′ |
| Myc | F-5′gagctggaatctcgagtgtaag-3′ |
| | R-5′agccaaggttgtgaggttag-3′ |
| B2m | F-5′cagttccacccacctcaaatag-3′ |
| | R-5′gtgtgagccaggatgtagaaag-3′ |
| Sox2 | F-5′taagactagggctgggagaa-3′ |
| | R-5′gccgcgattgttgtgattag-3′ |
| Nanog | F-5′gaagatgcggactgtgttct-3′ |
| | R-5′gctcaggttcagaatggtagag-3′ |
| Klf4 | F-5′gccaggagagagttcagtattt-3′ |
| | R-5′ctgacttgctgggaacttga-3′ |

Overexpression of Candidate Genes (PRRX1 and SNAI2) in the HF Hybrids. Expression vectors containing full length rat genes SNAI2 and PRRX1 (Origene, Inc.) were introduced into the hybrid cell line HF by lipofection using Lipofectamine Plus reagent (Invitrogen, Inc.) according to manufacturer's protocol. After 2-3 weeks, stable G418 resistant clones (HF-SNAI2 and HF-PRRX1, respectively) were selected, expanded, and used for subsequent experiments.

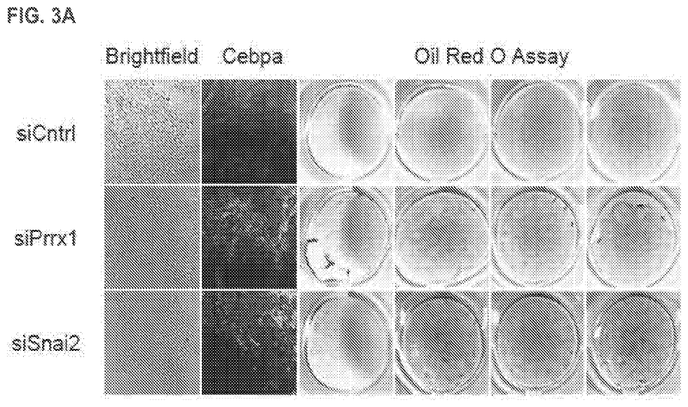

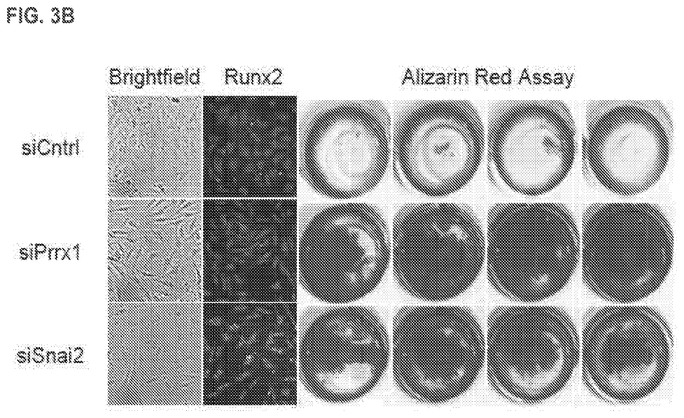

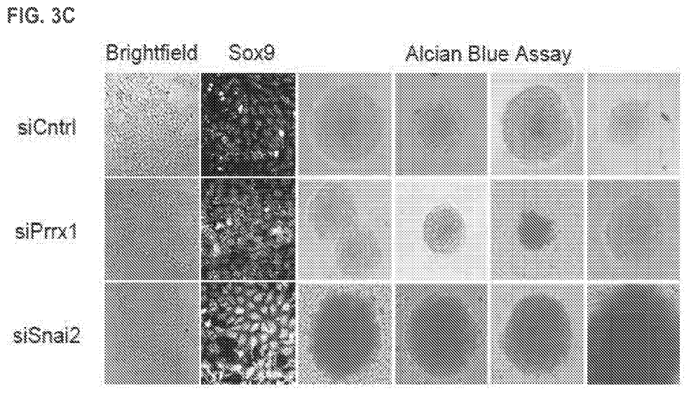

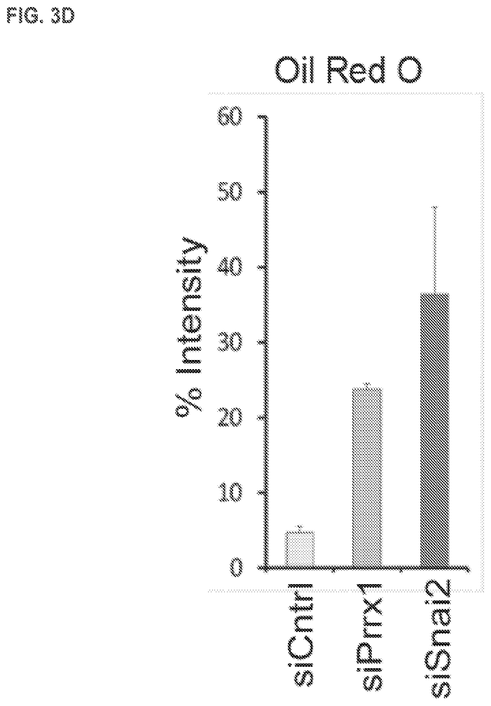

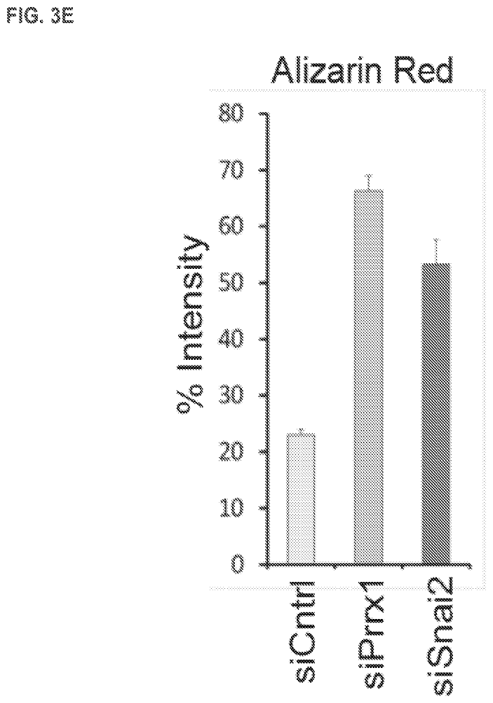

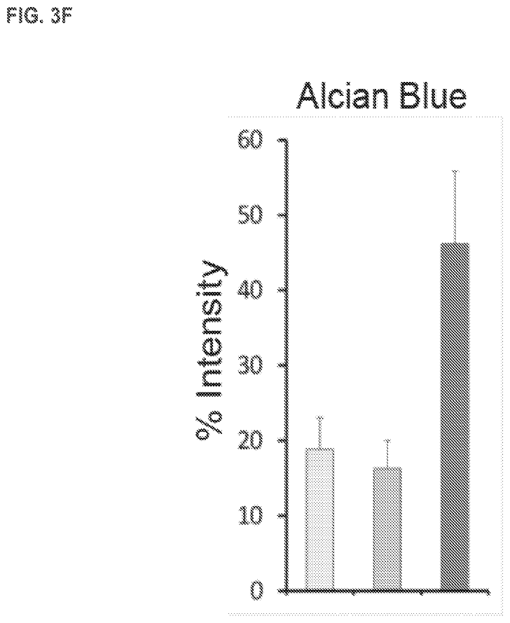

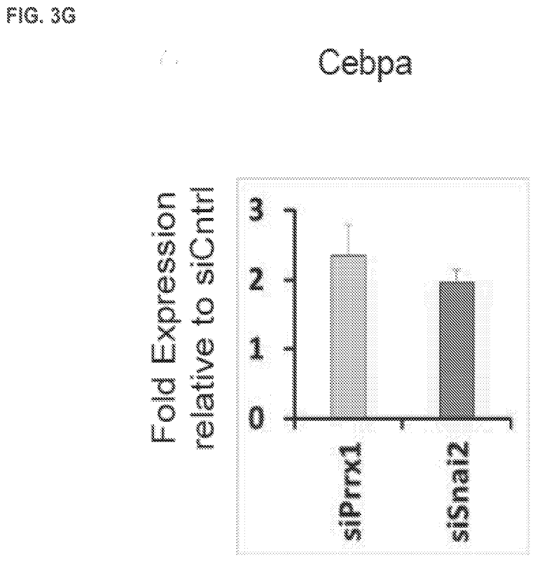

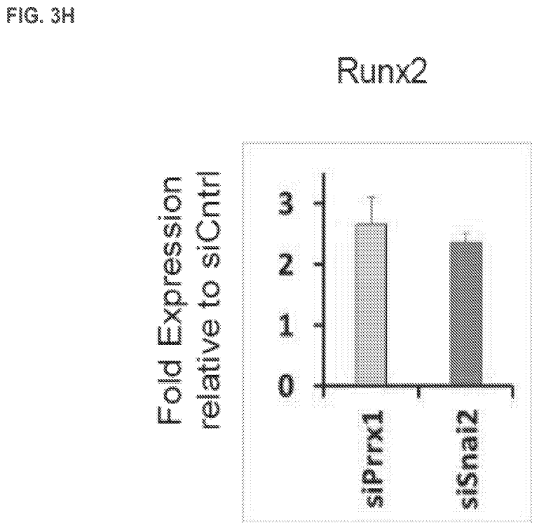

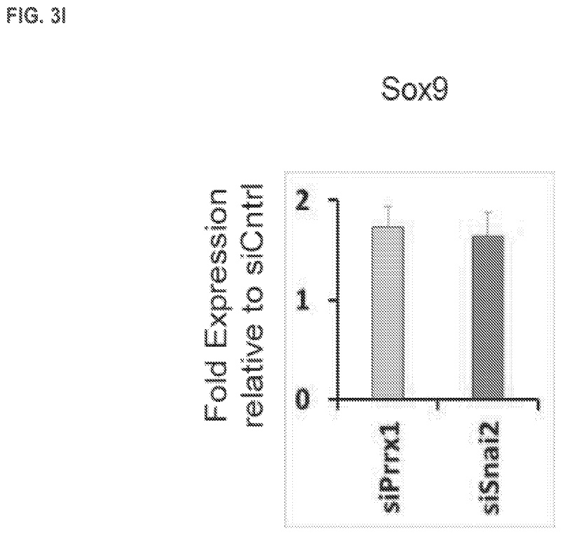

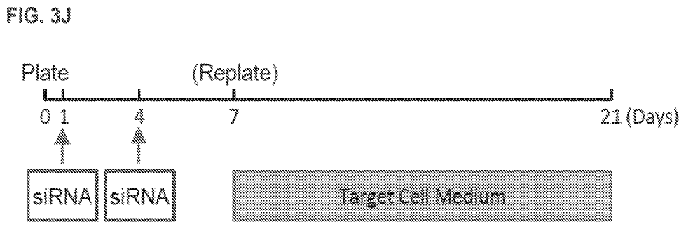

Knockdown of Candidate Genes (PRRX1 and SNAI2) in the REF Cells. RAT-2 REFs (acquired from ATCC) were seeded (four replicates) in 6-well cell culture plate (Nunc) at a density of 2×10⁶ cells/well and incubated at 37° C. in a CO2 incubator one day before transfection was performed with 50 nM (final concentration) of pooled small interfering RNA (siRNA, Santa Cruz Biotech), 2.5 μl of Lipofectamine RNAiMAX (Invitrogen, USA) and Opti-MEM (Invitrogen, USA) according to the manufacturer's instructions. RNAi negative universal control MED (Invitrogen) was used to calibrate siRNA transfection. Repeat transfection was performed on Day 4 in order to achieve sustained inhibition, prior to using cells for subsequent experiments on Day 7 (FIG. 3J).

Cell Migration Assay. Trans-well cell migration assay for REFs, HFs, HF-SNAI2 and HF-PRRX1 cells was carried out using 24-well cell migration assay from Trevigen (Catalog #3465-024-K) following manufacturers protocol (n=3 biologic replicates for each experimental group). FTO2-B Rat hepatoma (RH) cells, RAT-2 fibroblast (REF) cells, hybrid (HF) cells, and SNAI2 and PRRX1 overexpressed HF clones (HF-SNAI2 and HF-PRRX1, respectively) were re-suspended at the concentration of 1×10⁶ cells/ml in the migration buffer consisting of FDV medium and 0.1% BSA without serum. Conditioned FDV media containing 10% fetal bovine serum was used as the chemo-attractant. 100 μl of cells was added on top of the trans-well membrane in the upper chamber and 600 μl of FBS containing (chemo-attractant) FDV media was added to the lower chamber. The same volume of FDV media without serum was added to the control wells as a negative control. The cells were incubated at 37° C. in CO₂ incubator for 30 h. After incubation, the media in the top chamber was carefully aspirated without puncturing the membrane, and each well was washed with 100 μl of warm (37° C.) 1× Wash Buffer provided in the kit. The bottom chamber was aspirated and washed twice with 500 μl warm (37° C.) 1× Wash Buffer. Next, 12 μl of Calcein AM solution (supplied in the kit) was added to 12 mL of 1× Cell Dissociation Solution (provided in the kit). 500 μl of Cell Dissociation Solution/Calcein AM was added to the bottom chamber of each well. The chambers were reassembled and incubated at 37° C. in a CO₂ incubator for 1 h. The chambers were next disassembled by removing the inserts, and the assay chamber solutions at the bottom of the assay plate were read using a Biotek Synergy HT plate reader at 485 nm excitation, 520 nm emission. The experimental data, in terms of relative fluorescence units (RFU) was converted into cell numbers to determine the number of cells that have migrated.

Adipogenic induction. REF cells were cultured in a DMEM media with 10% FBS and 1% Pen-Strep. When the cells reached 70% confluency, they were trypsinized, pelleted and counted. 30000 cells were plated in 4 wells for each experimental group (siCntrIREF, siPrrx1REF and siSnai2REF) in a 96 well plate (n=4 biologic replicates for each experimental group). The wells were transfected with control, PRRX1 and SNAI2 siRNA. Seven days post initial siRNA transfection, transfected cells were treated with adipogenic induction medium (A) for three days (Cyagen Biosciences, Cat #GUXMX-90031) followed by adipogenic maintenance medium (B) for 11 days. On the 14th day, oil red staining was performed to assess adipogenic activity and cell pellets were preserved for further downstream analyses.

Oil Red O staining. Two weeks post adipogenic induction, cells were rinsed with phosphate buffered saline (PBS) and fixed with 4% paraformaldehyde (Sigma Aldrich) in PBS. The formaldehyde solution was removed by tilting the plate and rinsed with sterile water. Each well was then covered with 60% of isopropanol and incubated for 5 minutes. Isopropanol solution was pipetted out and 2 ml of working solution of Oil Red O (Sigma Cat #01391) was added in each well and incubated for 20 minutes at room temperature. Wells were then washed with water until the water ran clear. All wells were kept wet with water and viewed under the microscope. Lipid droplets were stained red with Oil Red 0. To record the OD readings Isopropanol solution was pipetted into a fresh 96 wells microtiter plate (Corning NBS Microplate) and OD reading was recorded at 490/500/510 nm, respectively.

Osteogenic induction. REF cells were cultured in a DMEM media with 10% FBS and 1% Pen-Strep. When the cells reached 70% confluency, they were trypsinized, pelleted and counted. 30000 cells were plated in 4 wells for each experimental group (siCntrIREF, siPrrx1REF and siSnai2REF) in a 96 well plate (n=4 biologic replicates for each experimental group). The wells were transfected with control, PRRX1 and SNAI2 siRNA. Seven days post initial siRNA transfection, transfected cells were placed in osteogenic medium (DMEM, FBS 10%, 50 μg/ml ascorbic acid, 10 mM β-glycerophosphate, 10 nM dexamethasone). Every 3 days old media was withdrawn and new Osteogenic media was added. After 14 days, cells were washed in cold PBS, fixed with 4% PFA in PBS and stained with 40 mM Alizarin Red that stained for Calcium to ascertain osteogenic activity and cell pellets were preserved for further downstream analyses.

Alizarin Red staining. Two weeks post osteogenic induction, cells were rinsed with phosphate buffered saline (PBS) thrice and fixed with 4% paraformaldehyde (Sigma Aldrich) in PBS for 15 min at room temperature. The formaldehyde solution was removed, and the cells rinsed with sterile diH2O. Water was removed completely and 1 mL of 40 mM Alizarin Red S (ARS) was added and alizarin red staining was carried out as per manufacturer's protocol (ScienCell, ARS Staining Quantification Assay ARed-Q Catalog #8678).

Chondrogenic induction. REF cells were cultured in a DMEM media with 10% FBS and 1 Pen-Strep. When the cells reached 70% confluency, they were trypsinized, pelleted and counted. 30000 cells were plated in 4 wells for each experimental group (siCntrIREF, siPrrx1REF and siSnai2REF) in a 96 well plate (n=4 biologic replicates for each experimental group). The wells were transfected with control, PRRX1 and SNAI2 siRNA. Seven days post initial siRNA transfection, transfected cells were placed in chondrogenic medium (MSC go Chondrogenic XF™, Biological Industries) and incubated for 14 days in incubator (37° C., 5% CO₂). The differentiation medium was changed every 3 days for 14 days following which Alcian Blue staining was used to evaluate chondrogenic activity and cell pellets were preserved for further downstream analyses.

Alcian Blue staining. Two weeks post osteogenic induction, cells were rinsed with phosphate buffered saline (PBS) thrice and fixed with 4% paraformaldehyde (Sigma Aldrich) in PBS for 15 min at room temperature. The formaldehyde solution was removed, and the cells rinsed with sterile diH2O. Water was removed completely and 0.2 ml of 1% Alcian Blue solution (Sigma; A-3157) was added to each well and Alcian blue staining carried out as per kit manufacturers protocol (MSC go Chondrogenic XF™, Biological Industries).

Generation of dMSCs. REFs were cultured in a DMEM media with 10% FBS and 1% Pen-Strep. When the cells reached 70% confluency, they were trypsinized, pelleted and counted. 30000 cells were plated in 4 wells for each experimental group (siCntrIREF, siPrrx1REF and siSnai2REF) in a 96 well plate (n=4 biologic replicates for each experimental group). The wells were transfected with control, PRRX1 and SNAI2 siRNA. Seven days post initial siRNA transfection, transfected cells were placed in Rat MSC medium (Rat MSC growth medium kit, Cell Applications, Inc.) and incubated for 14 days in incubator (37° C., 5% CO2). The MSC medium was changed every 3 days for 14 days following which Alkaline Phosphatase Assay (Anaspec EGT, Catalog #AS-72146) was used to assess mesenchymal stem cell activity according to manufacturer's instructions and cell pellets were preserved for further downstream analyses.

Generation of dPSCs and Embryoid bodies. All three groups (siCntrIREF, siPrrx1REF siSnai2REF) of rat embryonic fibroblasts (REF) cells were transferred (seven days post initial siRNA transfection) onto REF feeder layer inactivated with mitomycin C (Sigma) (n=4 biologic replicates for each experimental group) in replicates of four in a 12 well plate and maintained in Rat ESC media (DMEM/F12 supplemented with 20% knockout serum replacement (KSR) and 8 ng/mL of fibroblast growth factor (FGF-2) for 14 days in incubator (37° C., 5% CO₂) (Gomes et al. 2010). After 14 days, the generated dPSCs were transferred to non-adherent dishes and cultured in suspension culture for 8 days in differentiation media to induce Embryoid Body (EB) formation. Cell pellets from matching experiments were preserved for further downstream analyses. dPSCs were differentiated into each of the three germ layers according to the Human Pluripotent Stem Cell Functional Identification Kit (R&D Systems®, Catalog #SC027B).

Fixation and Cryopreservation of Embryoid Bodies. The EBs were collected from the culture dish with a pipette and transferred to a 15 mL conical tube until all material sank to the bottom of the tube. After gently removing the media, the EBs were fixed with a 4% paraformaldehyde (PFA) solution in PBS for 30 minutes in room temperature. Next the PFA solution was removed and the cells washed with PBS for 5 min.

Immunofluorescence Staining. Immunofluorescence staining was performed on Hepatoma (FT02B), Rat embryonic Fibroblast (REF), Hepatoma-Fibroblast Hybrid (HF)) cells, and PRRX1 and SNAI2 overexpressed HF clones (HF-PRRX1 and HF-SNAI2) grown on 12-mm glass coverslips. At the appropriate time points, coverslips were removed, washed in ice-cold BSA/PBS before fixation in 4% paraformaldehyde (PFA). Cells were then permeabilized with 0.1% Triton X-100 and incubated with PBSX containing 1% (w/v) donkey serum for 2 hours to block all existing rat binding sites and incubated overnight at 4° C. in a humid chamber with appropriate monoclonal primary antibody [PRRX1 (Novus Biologicals), SNAI2 (SantaCruz Biotechnologies), Col1a1 (Origene), Sox2 (Invitrogen USA), Nanog (Invitrogen USA), Myc (SantaCruz Biotechnologies)] used at concentrations of 5 μg/ml] followed by labeling with Alexafluor-conjugated secondary antibody (1:100 dilution). Coverslips containing the cells were then incubated for 10 minutes in dark at room temperature with working solution of 4′,6-diamidino-2-phenylindole (DAPI) (1 μg/mL), washed twice with DI water to remove residual salts. Samples were subsequently mounted on top of the drop of Prolong Antefade gold mounting media (Life Technologies) and analyzed with a fluorescent microscope (Leica DMIL LED) using 20× magnification and appropriate wavelength settings.

Immunofluorescence detection of three germ layers. The fixed samples were embedded in agarose to trap and immobilize the embryoids. Once the agarose solidified it was kept in 4° C. for hardening. The samples were then sent for further processing. The samples were embedded in paraffin wax and sectioned at 5-6μ thickness using a microtome. The sections were then adhered to positively charged slides and then dewaxed in Histoclear and rehydrated as per protocol (Sivaguru et al., 2013). The rehydrated slides were permeabilized with 0.5% TritonX-100 in phosphate buffered saline and labeled with human three germ layer 3-color immunocytochemistry kit from R and D biosystems following the manufacturer protocol (Catalog #SC022, R and D systems, Inc., MN, USA). Briefly, the sections were blocked by 10% normal donkey serum (Jackson Laboratories, USA) containing 1% BSA (Thermofisher, USA). After blocking the sections were incubated with a combination of two conjugated primary antibodies for each lineage of interest such as, ectoderm (Anti-Sox1 conjugated with Northern Lights 493 and anti-Otx-2 conjugated with Northern Lights 557), mesoderm (Anti-Brachyury Northern Lights 557 and anti-Hand1 conjugated with Northern Lights 637), and endoderm (Anti-Gata-4 conjugated with Northern Lights 493 and Anti-Sox17 conjugated with Northern Lights 637). Each combination was incubated for 3 h at room temperature in the dark, washed with PBS containing BSA, counterstained with Hoechst nuclear dye (Thermofisher, USA), mounted in Prolong Gold antifade mounting media (Thermofisher, USA), left for 24 h at room temp and stored in 4° C. after sealing with nail polish.

Airyscan Super-Resolution Imaging. The labeled sections were imaged under a Zeiss LSM 880 Laser Scanning Microscope with Airyscan Super-Resolution system. Excitation and emission wavelengths that were collected include: 405 nm excitation (emission collected between 410-460 nm), 488 nm excitation (emission collected between 500-550 nm), 561 nm excitation (emission collected between 570-615 nm) and 633 nm excitation (emission collected between 650-700 nm). An internal GaAsP photo-multiplier tube detector was used to scan entire stone thin sections using a 63× Plan Apochromat (NA 1.4 oil immersion objective. Several images were taken at different sections and locations for each sample. The images were recorded at a super-resolution sampling frequency of 40 nm per pixel. The raw data were processed for super-resolution in the same program Zeiss Zen software and pseudo colored for visualizing four channels.

Statistics and Reproducibility. All in vitro transdifferentiation and reprogramming related experiments were repeated independently at least three times with similar results. Details on sample sizes and reproducibility are provided in the respective Methods section headers. Wherever appropriate, data are presented as the mean±s.e.m. Wherever appropriate, comparisons between groups used the Student's t-test assuming two-tailed distributions.

Generation of Somatic Cell Hybrids and Identification of Lineage Defining Transcription Factors

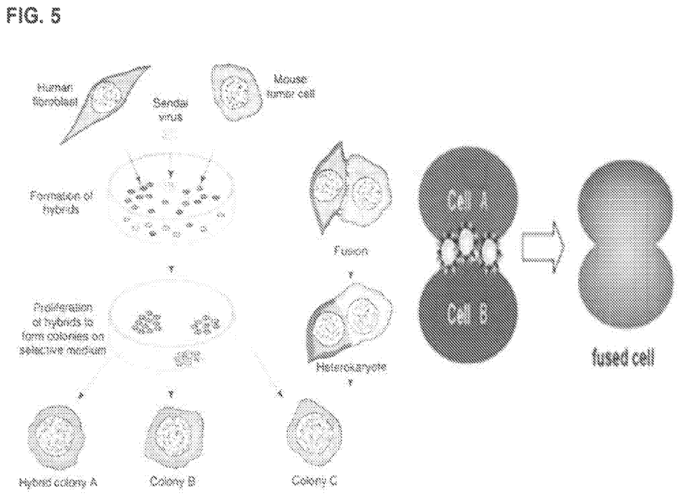

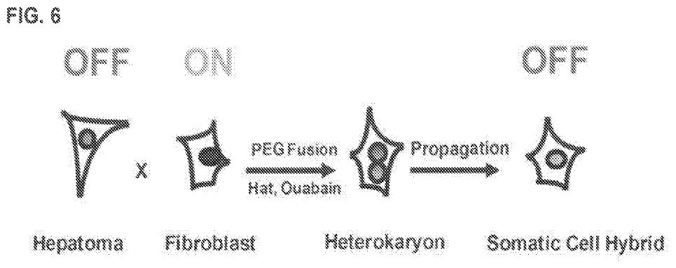

In order to map transcriptional regulatory circuits that mediate cell differentiation and gene extinction, Next generation sequencing (NGS) that includes powerful techniques like whole genome microarrays (Shendure & Aiden 2012), DNA sequencing (Ozsolak & Milos 2011), RNA sequencing (Zhao et al. 2008), chromatin profiling (Furey 2012), and chromatin immunoprecipitation (ChIP) assays (Carey et al. 2009) are increasingly being used. In addition, somatic cell hybridization techniques discovered by Barski et al. (Barski et al. 1960; Sorieul & Ephrussi 1961) were among the first techniques employed to investigate the molecular mechanisms underlying tissue specific gene expression (see FIG. 5). Since spontaneous fusion of cells occurs at a very low frequency, sendai virus or polyethylene glycol (PEG) is used as a fusogen (Kao et al. 1974). The initial product of fusion contains within a common cytoplasm, two or more distinct nuclei from both parent (heterokaryon). Only a small portion of these heterokaryons progress to nuclear fusion and mitosis. The best-known media for selecting the hybrids include Hypoxanthine+Aminopterin+Thymidine (HAT) medium. Aminopterin stops the biosynthesis of purines and pyrimidines using simple sugars and amino acids. Hypoxanthine present in the medium is converted into guanine by the help of an enzyme known as hypoxanthineguaninephosphoribosyltransferase (HGPRT) while phosphorylation of thymidine is catalyzed by thymidine kinase (TK). Only cells with active HGPRT and TK enzymes can survive and proliferate in HAT medium. To use HAT as a selective medium, the parental cells must be deficient in any one of these two enzymes while the somatic hybrid cells should have both these enzymes in active state that hence will help the hybrids survive in HAT medium.

Somatic cell hybridization may be a useful approach to understanding the molecular mechanisms of repression of lineage appropriate genes because the hybrid system takes into account the net effect of gene regulatory mechanisms of each parental cell line and displays the activated or repressed states of lineage specific genes from a particular parental cell. No other approach at present can be used to simultaneously study the net result of multiple gene regulatory mechanisms on a global scale without introducing genetically engineered modifications.

Using the somatic cell hybridization technique, extinction of lineage-specific genes has been studied in liver, muscle, myeloid, and pituitary cells (Ringertz & Savage 1976). In particular, extinction of liver-specific gene expression in hepatoma X fibroblast hybrids has been extensively characterized (Killary & Fournier 1984; Killary et al. 1984) (see FIG. 6). Several important regulatory transcription factors have been identified in hepatoma cells that have contributory roles in maintaining the liver specific cell lineage (Bulla & Fournier 1994; Bulla et al. 2004; Bulla et al. 2010).

By comparison, much less work has been done on assessing gene regulatory networks involved in lineage specific gene extinction in fibroblasts. Fibroblasts represent heterogeneous mesenchymal progenitor cells which are extensively found in conjunction with almost all cell types as the primary interstitial cell support system (Strutz et al. 1995). Besides playing a supporting role, these cells play a role in the structural organization, locomotion, epithelial mesenchymal transition (Acloque et al. 2009). Fibroblastic connective tissue, collagen, constitutes 25-35% of total protein in a normal mammalian body. It is known that the origin of fibroblasts typically takes place during the embryological transition from epithelial to mesenchymal phenotype induced by transforming growth factors (TGFs) (Willis et al. 2005).

In order to study gene silencing, the somatic cell hybridization technique was employed to generate (hepatoma X fibroblast) FR(2) hybrids. Instead of utilizing interspecies hybrids, normally used for generating cell hybrids (due to ease of distinguishing chromosomes derived from each parental cell), rat hepatoma and rat fibroblast cells were used to generate FR(2) hybrids. This type of intraspecies hybrid has been highly characterized and proven to have a stable genotype that rarely demonstrates chromosome loss and makes a useful model in which to apply whole-genome microarray analysis to study the phenomenon of tissue specific gene extinction.

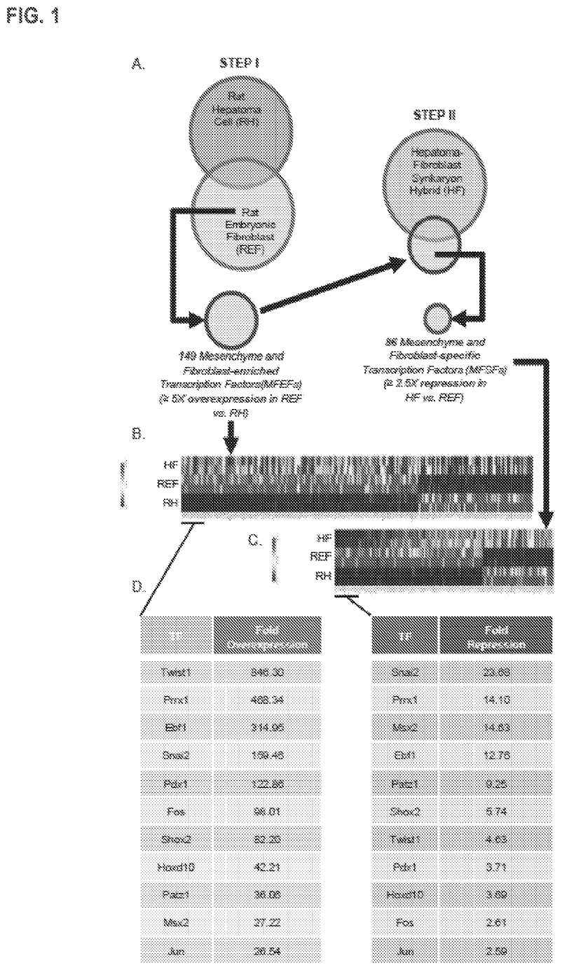

To identify the most critical lineage-defining TFs of REFs, an unbiased and comprehensive experimental discovery approach was pursued involving generation of such stable intra-species somatic cell synkaryon hybrids (HFs) created by fusing REFs of mesenchymal origin with rat hepatoma (RH) cells of endodermal origin after genetically engineering each cell line so that each possessed a unique yet separate xenobiotic resistance mechanism (Bulla et al. 2012). Only true synkaryon hybrid cells that survived exposure to both xenobiotics were selected while the residual parental cells that were incapable of surviving exposure to either one or the other xenobiotic perished. By subjecting the starting parental as well as resultant hybrid cells to genome-wide transcriptomic profiling, pivotal master regulatory TFs that enforce mesenchymal and fibroblast cell fate were identified. These TFs are not only the most important for lineage preservation and maintenance, but whose downregulation constitutes an imperative requirement for reacquisition of cellular plasticity by these cells. First, 149 TFs that demonstrated greater than 5-fold overexpression in REFs compared to RH cells were selected, which are referred to as mesenchymal fibroblast-enriched TFs (MFEFs) (FIG. 1A). The MFEFs were then screened to identify 86 TFs that demonstrated greater than 2.5-fold repression in hybrids compared to REFs and referred to these as mesenchymal fibroblast-specific TFs (MFSFs) (FIG. 1A). SNAI2 and PRRX1 were the top MFSFs that were repressed in hybrids compared to REFs (FIG. 1D).

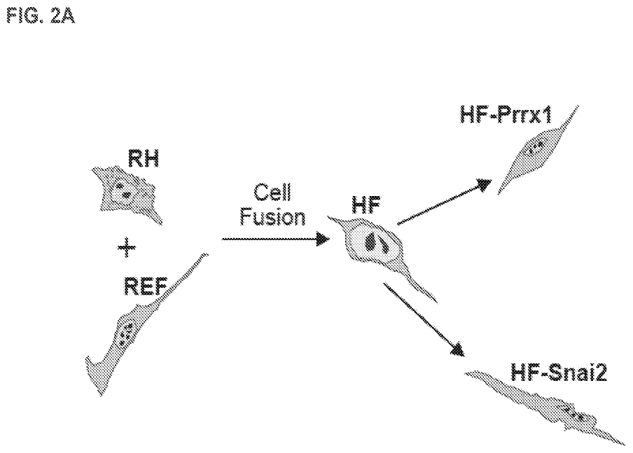

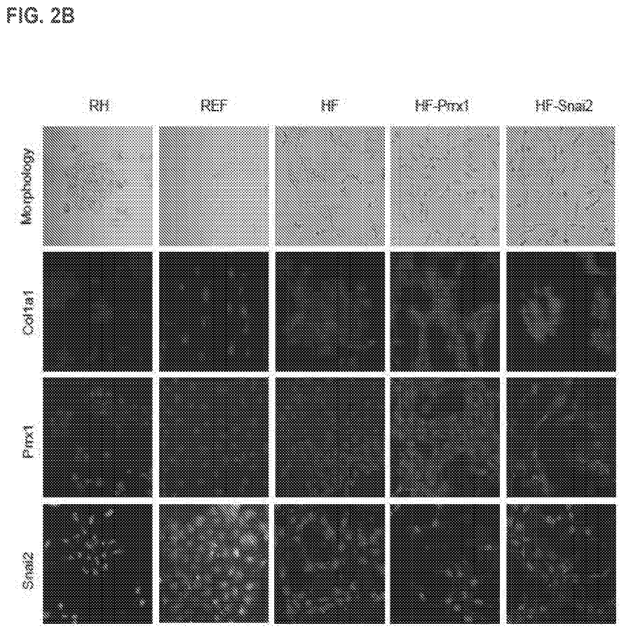

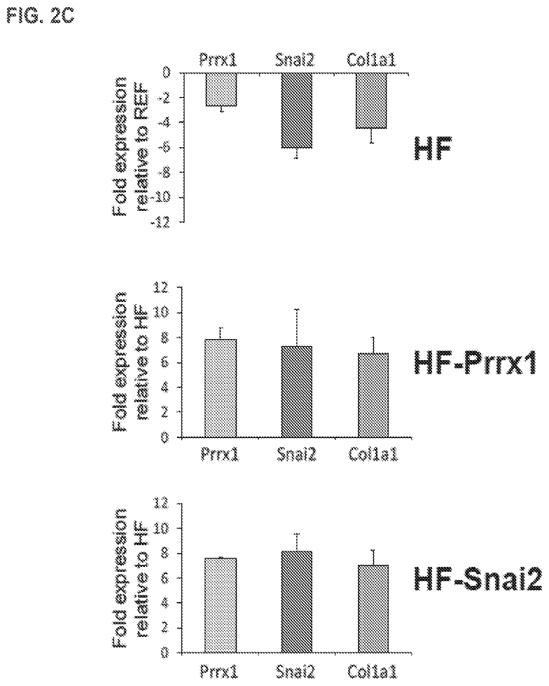

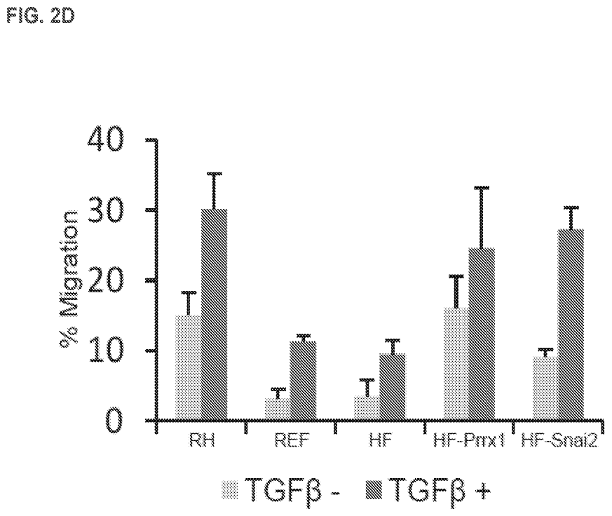

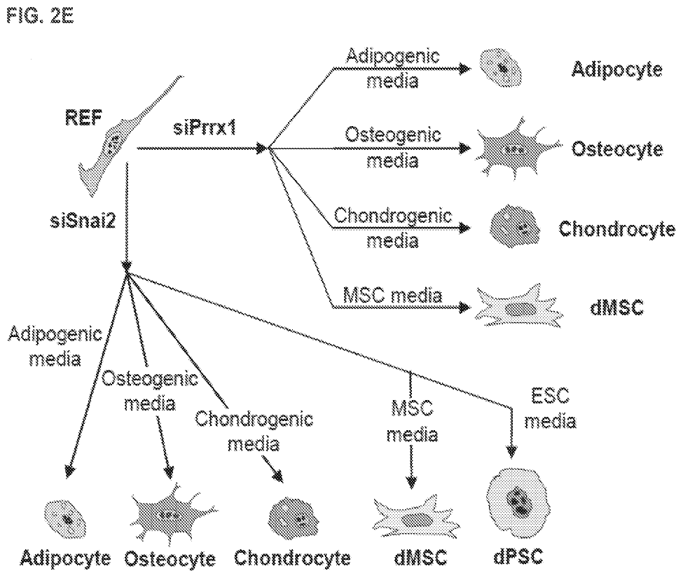

Next, expression of the candidate MFSFs identified from the microarray analysis was validated by quantitative reverse transcriptase polymerase chain reaction (qRT-PCR). Repression of fibroblast specific genes (PRRX1, SNAI2 and Col1a1) was also confirmed in somatic cell hybrids (FIG. 2C). To ensure the robustness of these identified MFSFs the top two MFSFs (SNAI2 and PRRX1) were ectopically overexpressed individually in the somatic cell hybrids. Generation of G418 resistant SNAI2 and PRRX1 overexpressed hybrid clones, respectively, led to re-acquisition of fibroblast traits (morphology, structure and functionality) in the hybrid cells. SNAI2 and PRRX1 clones attained fibroblast-like spindle-shaped morphology (FIG. 2A,2B), re-expressed fibroblast prototypical gene Col1a1 as confirmed by IF and qRT-PCR (FIG. 2B, 2C) and displayed significantly increased migration ability compared to the hybrids especially in response to TGF-Beta stimulation, a characteristic fibroblast-specific functional trait (Acharya et al. 2008) (FIG. 2D), while individual siRNA-mediated knockdown of SNAI2 and PRRX1 in REFs yielded siSnai2REF and siPrrx1 REF cells, respectively, with loss of spindle-shaped morphology, loss of Col1a1 expression and diminished migratory capacity compared to parental REFs.

Repressing an Intrinsic Lineage-Defining Transcription Factor Alone can Reprogram Somatic Cells

After confirming the pivotal importance of SNAI2 and PRRX1 in preserving the structural and functional identity of mesenchyme derived REFs, it was next determined whether the individual forced transient repression of either of these two critical TFs (utilizing a serial transfection protocol with siRNAs specific for either SNAI2 or PRRX1) could lift the cell fate restrictions tied to mesenchymal and fibroblast identity and render these cells more plastic and prone to cellular reprogramming. To test this, the siSnai2REF and siPrrx1REF cells were incubated in respective growth media of closely allied cells of mesenchymal lineage. When cultured in adipogenic media for 3 days followed by adipogenic maintenance media for 11 days, siSnai2REF and siPRRX1 REF cells attained lipid droplet-filled spherical morphology akin to adipocyte cells. Lipid vacuoles were visualized by light microscopy, stained red with Oil Red 0 (FIG. 3A, 3D) and these cells expressed adipocyte lineage-appropriate TF Cebpa as evaluated by qRT-PCR (FIG. 3G) and immunofluorescence (IF) (FIG. 3A). Similarly, individual knockdown of SNAI2 or PRRX1 in the parental REF by siRNA transfection and maintenance of the siSnai2REF and siPRRX1REF cells in either osteogenic or chondrogenic media for 14 days resulted in functional osteocytes and chondrocytes, respectively. Lineage-appropriate function of forming bone was evidenced by positive staining with Alizarin Red (FIG. 3B, 3E) while cartilage formation was confirmed by positive staining with Alcian blue, (FIG. 3C, 3F). Expression of osteocyte and chondrocyte lineage-instructive TFs Runx2 and Sox9, respectively, was also confirmed using qRT-PCR (FIG. 3H, 3I) and immunofluorescence (IF) (FIG. 3B, 3C).

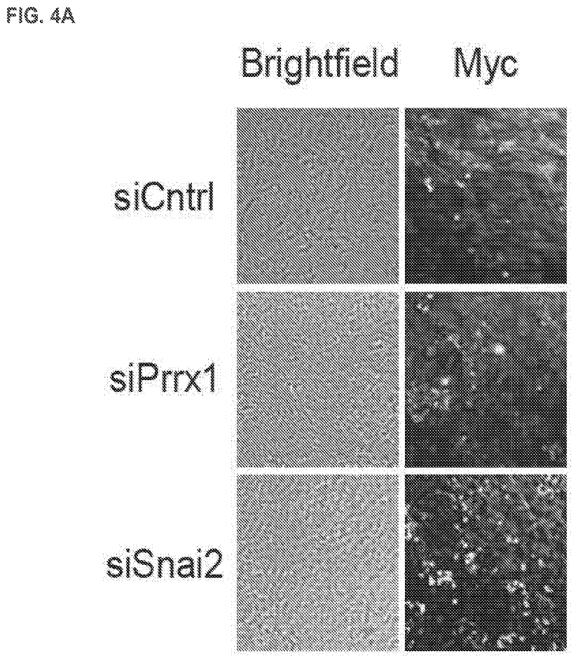

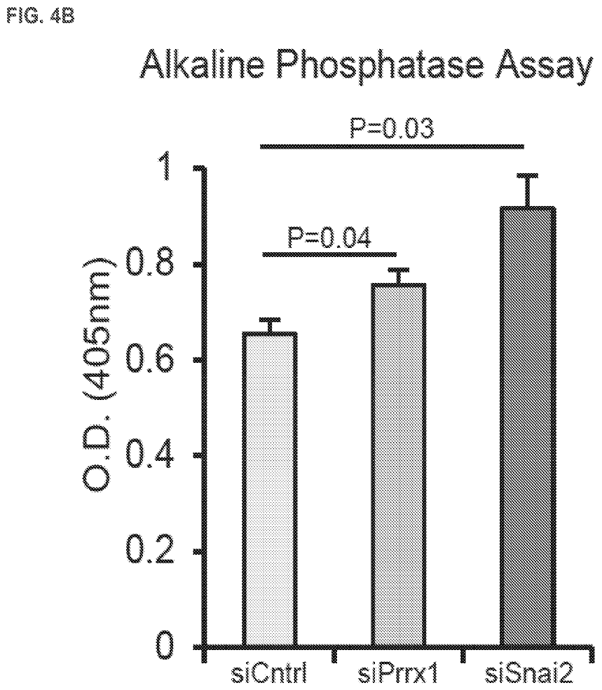

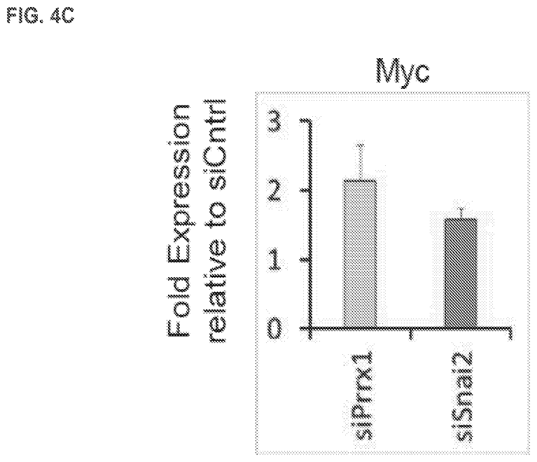

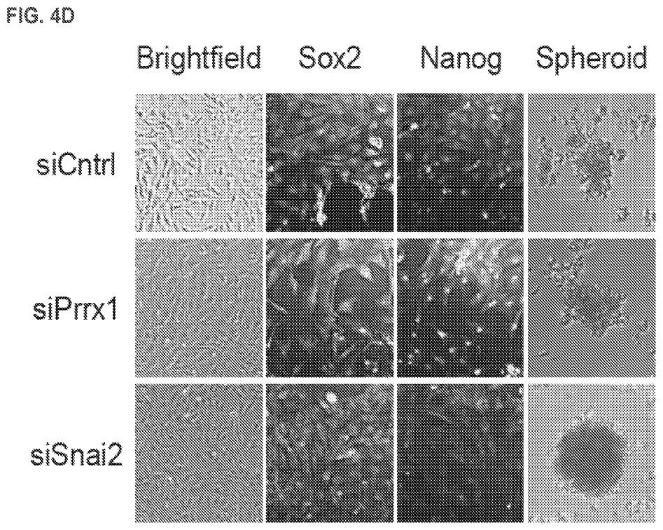

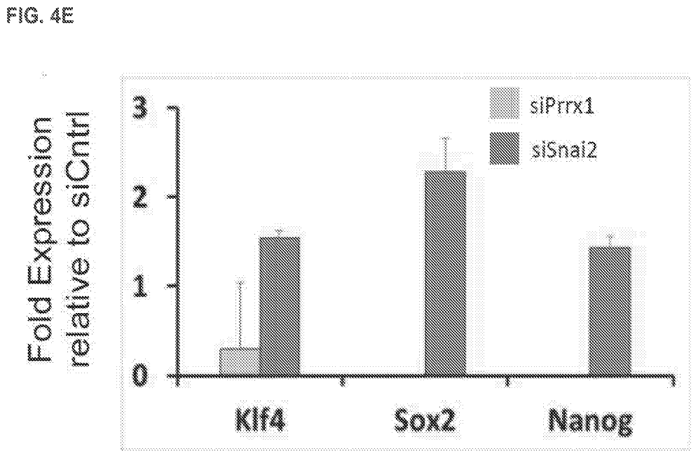

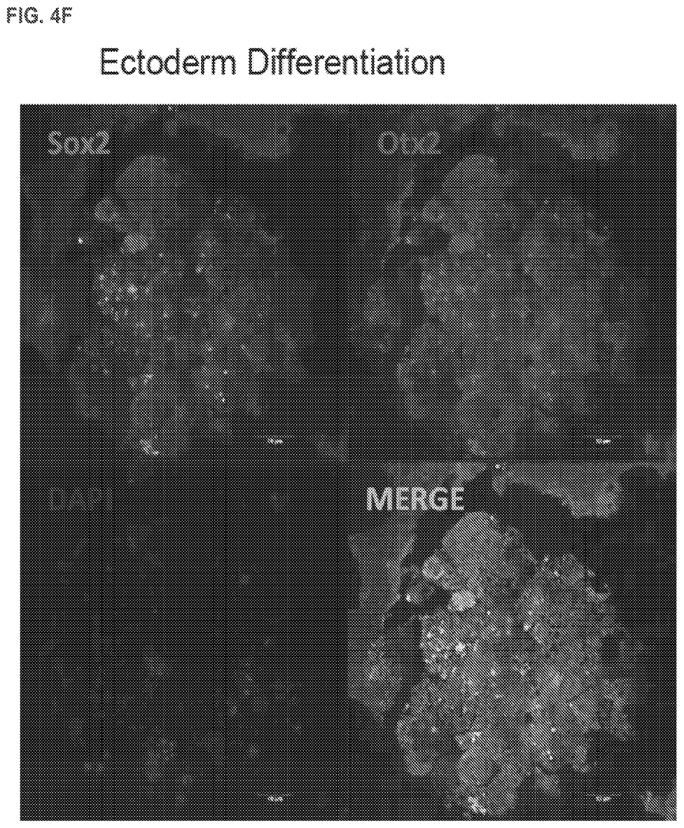

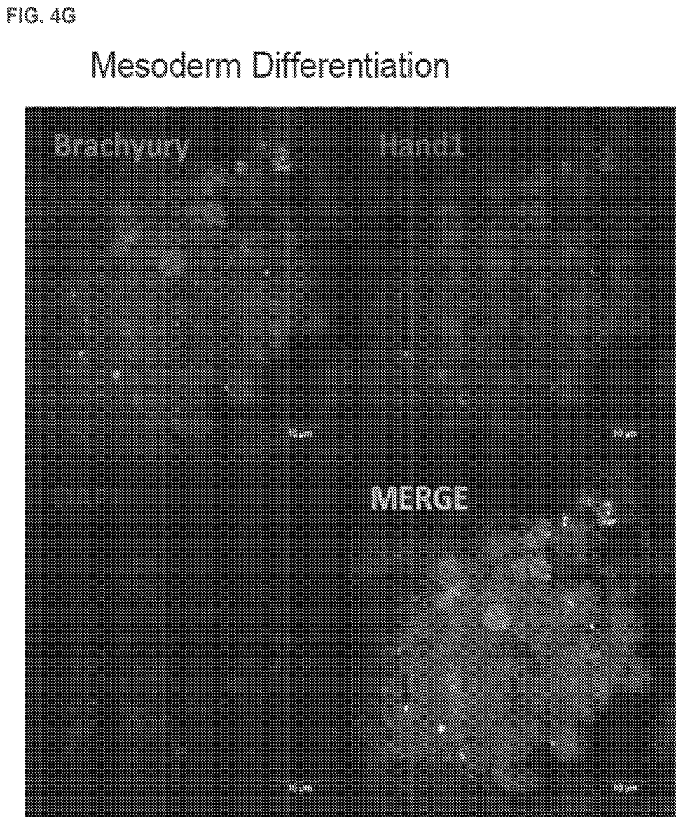

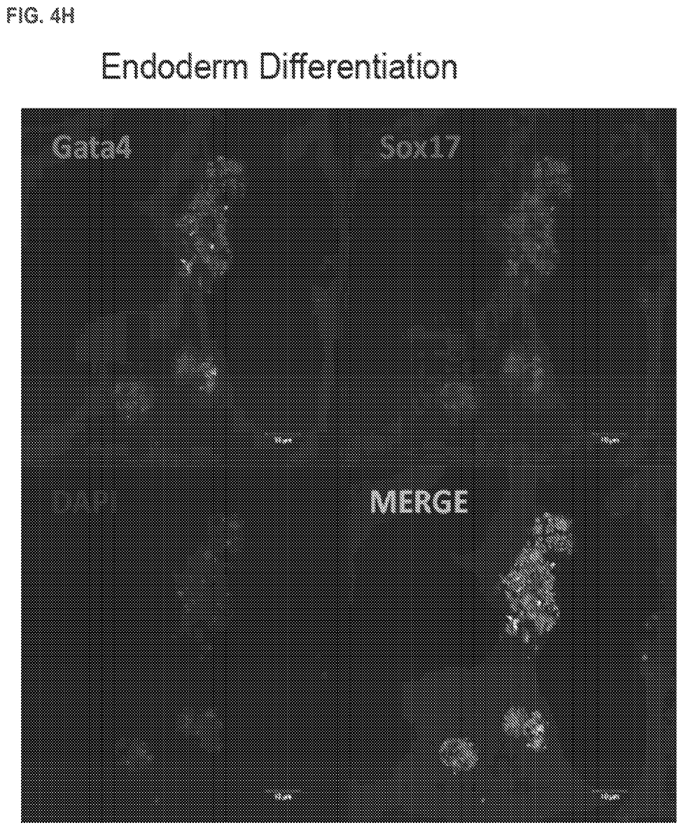

Next it was determined whether the transient repression of either SNAI2 or PRRX1 and incubation in suitable cell culture conditions could effectively transform parental fibroblasts into a more primitive precursor cell state, developmentally precedent to the mesenchymal differentiated state. siSnai2REF and siPRRX1REF cells when cultured in rat mesenchymal stem cell (rMSC) media for 14 days resulted in transformation to dedifferentiated multipotent stem cells (dMSCs), with altered morphology and enhanced expression of the prototypical MSC TF Myc (validated by both IF (FIG. 4A) and qRT-PCR (FIG. 4C). dMSCs showed significantly enhanced expression of alkaline phosphatase, an indicator of undifferentiated stem cell activity (FIG. 4B). When siSnai2REF and siPRRX1REF cells were cultured over mitomycin-C inactivated REFs in rat embryonic stem cell (rESC) media (with 2i/LIF) for 14 days (Jackson et al. 2010), attainment of dedifferentiated pluripotent stem cell traits occurred only in the siSnai2REF group (FIG. 4D) with expression of characteristic pluripotency factors like Sox2, Klf4 and Nanog, (FIG. 4E, 4F). Since these cells were obtained by lifting reprogramming barriers (lineage preserving TFs) that impede dedifferentiation, we refer to these cells as dedifferentiated pluripotent stem cells (dPSCs). Suspension culture of dPSCs, when placed in rESC differentiation media for 8 days, formed embryoid bodies that comprised of all three germ layers and stained positive for triple germ layer-specific TFs namely Sox2 and Otx2 (ectoderm) (FIG. 4F), Brachyury and Hand1 (mesoderm) (FIG. 4G) and Gata4 and Sox17(endoderm) (FIG. 4H) as validated by IF staining and confocal microscopy.

Cell specific identity is decided by the expression of cell type specific transcription factors and the underlying epigenetic state. TF mediated somatic cell reprogramming (whereby target cell-specific TFs are ectopically overexpressed) and small molecule driven reprogramming (whereby pathway modulators induce changes in the epigenetic landscapes) are both powerful tools employed to generate iPSCs or differentiated cells of desired lineages. However, a fundamental drawback of such protocols remains the low reprogramming efficiency and generation of partially transformed cells predominantly due to retention of the starting cells' transcriptional network and residual epigenetic memory (Nashun et al. 2015). Studies have shown that in such intermediately transformed cells, some specific genes of the starting cells are persistently expressed, whose siRNA mediated knockdown considerably improved reprogramming efficiency (Mikkelsen et al. 2008, Ebrahimi et al. 2015). Though multiple gene knockdown of the starting fibroblast cell's Transcriptional Regulatory Network (TRN) yielded differentiated adipocytes (Tomaru et al. 2014), transdifferentiation was never achieved through repression of a single critical lineage instructive TF of the starting cell. Similarly, while TFs that interfere with dedifferentiation have been demonstrated to induce cell-type specific transcriptional profiles, repression of the identified TFs did not permit dedifferentiation (Hikichi et al. 2013). While it was noted that SNAI2 was among the earliest genes to be repressed during traditional OSKM mediated somatic reprogramming efforts (Mikkelsen et al. 2008, Polo et al. 2012, Cachiarelli et al. 2015), and that induced repression of PRRX1 or SNAI2 enhanced both OSKM (Yang et al. 2011) and Nanog (Gingold et al. 2014) mediated somatic reprogramming, respectively, there are no known prior studies that report successful transdifferentation to alternate cell fates, or dedifferentiation to multipotent/pluripotent primitive cell states by repressing a single lineage-preserving instructive TF, in the absence of any exogenous transdifferentiation or pluripotency TFs (e.g., OSKM) or small molecule cocktails. This is because lineage commitment, preservation and maintenance are known to be regulated by the starting cell's TRN comprising of multiple lineage-specific TFs working in conjunction to exert transcriptomic and epigenetic control, to enforce and maintain the cell lineage. Consequently, the existence of a of single pivotal “gatekeeper” TFs (GTFs) within the TRN of a lineage-committed cell which could single-handedly regulate the switch between lineage preservation and cell plasticity has never been demonstrated, and the concept that an important biologic function like enforcement and preservation of lineage preservation (after lineage commitment has already occurred) can be disrupted by manipulating a single key TF member of the starting cell's TRN was not thought to be possible.

Using REFs as a model system, these results show that transient repression of a single pivotal lineage-preserving TF of the starting cell (SNAI2 or PRRX1 in this case) can overcome the lineage preservation effect or reprogramming barrier imposed by the starting cells' TRN, rendering the cells more plastic, and if incubated in appropriate media, can lead to direct lineage conversion of the starting cell to alternate cell types (such as adipocytes, chondrocytes or osteocytes). While repression of either PRRX1 or SNAI2 was capable of dedifferentiating REFs to multipotent dMSCs, SNAI2 in all probability exerts its control at an even more primitive stage than PRRX1 in the hierarchy of mesenchymal cell development from pluripotent precursors as evidenced by the ability of siSnai2 to dedifferentiate REFs to dPSCs. This may be due to Snai2 first exerting transcriptional regulatory control in Day 3.5 CD326-CD56+embryonic mesodermal precursor cells (160× higher expression than ESCs), which precedes the first expression of Prrx1 (more a mesenchymal sublineage-specific TF), in the hierarchy of mesodermal lineage development (Evseenko et al., 2010). The described method resets a cell to an earlier point in its developmental timeline, and its transient nature likely allows for the derepression of plasticity maintenance factors (e.g., pluripotency TFs), enabling its innate differentiation mechanism to then proceed as guided by components provided in its growth medium. The detailed molecular mechanisms underlying the cellular transformations brought about by transient repression of SNAI2 and PRRX1 remain unknown as do any potential chromatin landscape alterations that may have resulted from such of RNAi manipulation. In conclusion, siRNA mediated transient repression of a somatic cell's lineage-defining TF is a tenable strategy to help achieve cell identity switch to alternate transdifferentiated cell fates or to dedifferentiated multipotent or pluripotent cell states. This may potentially enhance reprogramming efficiencies of TF/small molecule driven protocols both in terms of cell yield and process time and eventually help realize the goal of reliably obtaining clinically relevant somatic cells or multipotent/pluripotent stem cells for therapeutic use.

### Example 2: Human Mesoderm Lineage Development Confirms Rat Model and Establishes SNAI2 as a Gatekeeper LDTF

Among a cell's lineage-preserving TRN of TFs, there may exist specific “Gatekeeper” TFs which play such a critical role that they act as a molecular lynchpin, and their sole inhibition may singlehandedly permit cell reprogramming. It is important to note that lineage-specific TFs that are responsible for initial cell fate determination and lineage commitment, such as Eomes and Brachyury for mesoderm commitment, are known to repress pluripotency and neuroectoderm gene programs (Tosic et al., 2019). However, they are active at the mesendoderm stage (Tsankov et al., 2015) and have low to undetectable expression thereafter in downstream mesoderm-derived cells such as fibroblasts. They therefore are not accessible for siRNA-mediated inhibition as a method to derepress pluripotency factors and permit plasticity acquisition. The lineage-specific “Gatekeeper” TF that was sought must display expression in the differentiated cell and be accessible to siRNA-mediated inhibition and serve the purpose of lineage preservation and maintenance after lineage commitment has already occurred.

Confirmation of Rat Model

To assess whether the above findings in a rat in vitro model are relevant to and corroborated by human cell development and differentiation, the expression trends of mesenchymal fibroblast-specific genes (MFSGs) (SNAI2, PRRX1) and repression trends of embryonic stem cell genes (ESCGs, e.g. POU5F1, SOX2, NANOG, LIN28A) (Ben-Porath et al. 2008) across the spectrum of mesoderm lineage progression in 21 different human cells and tissues obtained from the publicly available ENCODE (Consortium, 2012; Davis et al., 2018) and ROADMAP (Roadmap Epigenomics et al., 2015) databases development were confirmed using RNA-Seq analysis.

Most members of the ESCG signature displayed strong expression in ESCs, weak to undetectable expression in fibroblasts, and an abrupt transition in expression profiles at the mesoderm stage of development. Most members of the MFSG signature displayed strong expression in fibroblasts, weak to undetectable expression in ESCs with a similarly abrupt transition in expression profiles at the mesoderm stage of development. As expected, SNAI2 and PRRX1 first displayed marked increase in their expression at the mesoderm stage. Based on the abrupt transition in gene expression profiles, efforts were focused on the mesoderm stage to try and uncover a mechanistic basis for these observations.

SNAI2 is a Gatekeeper

Enhancers are known to play an important regulatory role both in pluripotency gene regulation as well as in lineage-specific gene regulation (Gokbuget and Blelloch, 2019). Thus, studies were undertaken to identify enhancer chromatin modifications that occur at the mesoderm stage and their possible intersection with SNAI2 genomic targets in regulating the repression of ESCGs and the expression of MFSGs.

First, histone modification ChIP-Seq (H3K4me1, H3K4me3, H3K27ac, H3K27me3, H3K9me3) data from the publicly available ENCODE (Consortium, 2012; Davis et al., 2018) and ROADMAP (Roadmap Epigenomics et al., 2015) databases and defined active enhancers on the basis of overlapping H3K4me1 and H3K27ac peaks, and repressed enhancers on the basis of overlapping H3K4me1 and H3K27me3 peaks on ChIP-Seq analysis, using established methods (Creyghton et al., 2010; Zentner et al., 2011).

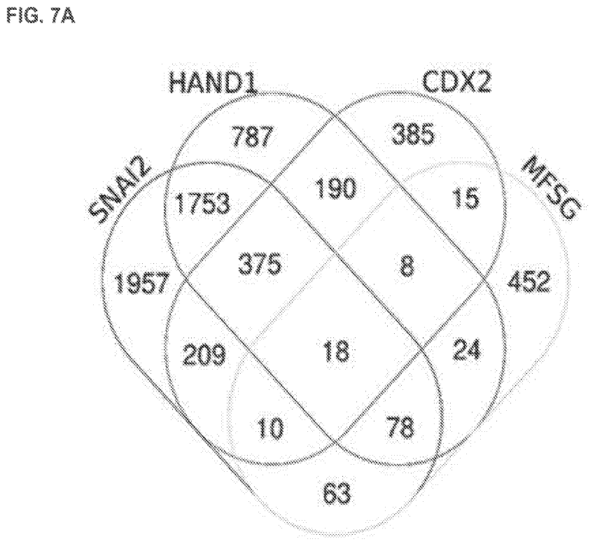

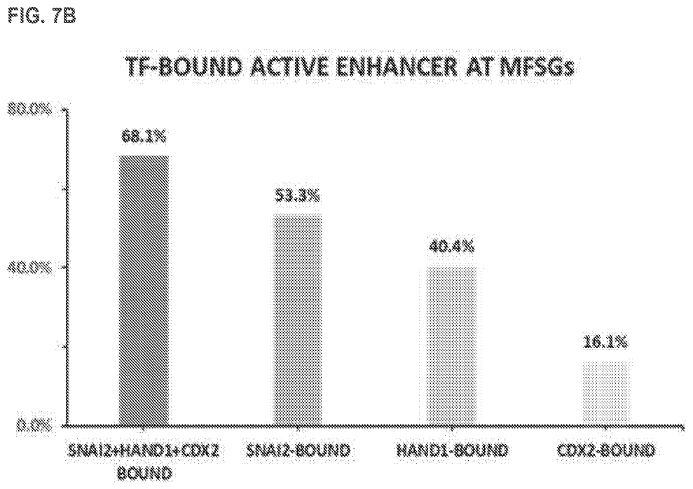

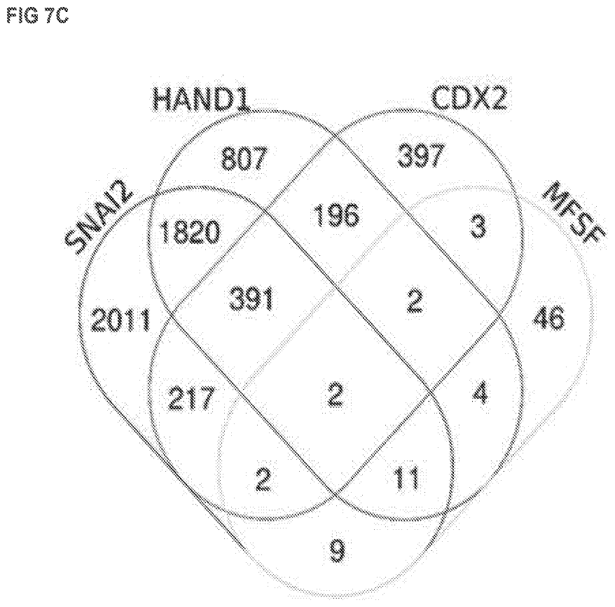

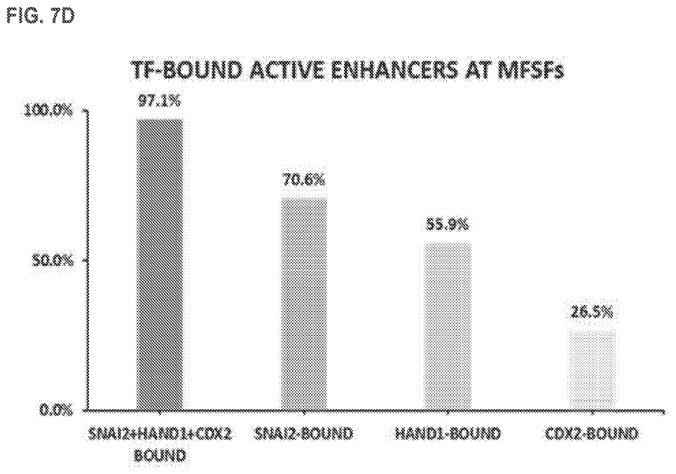

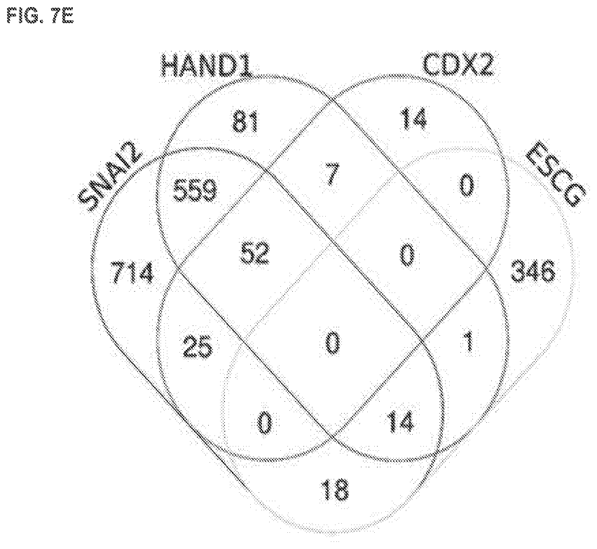

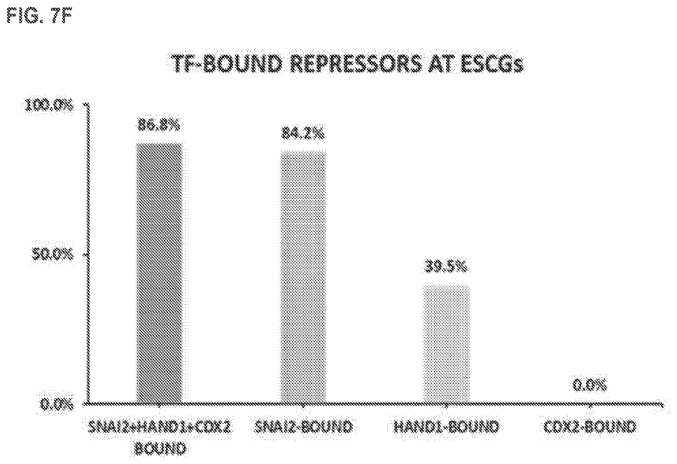

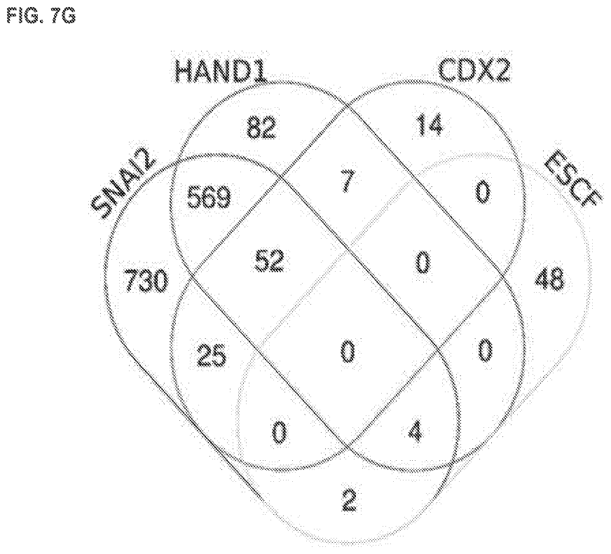

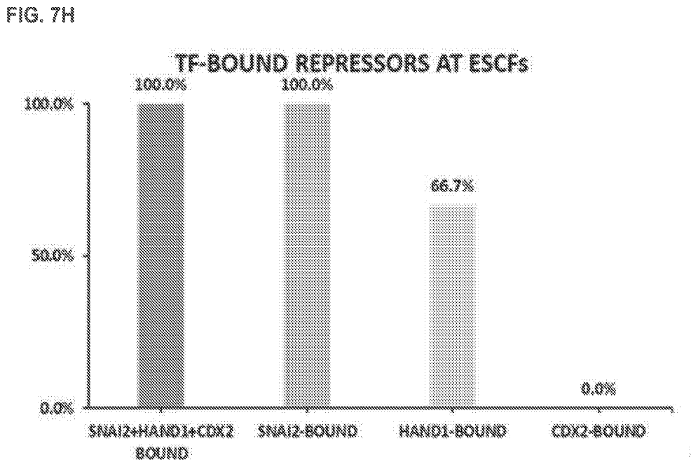

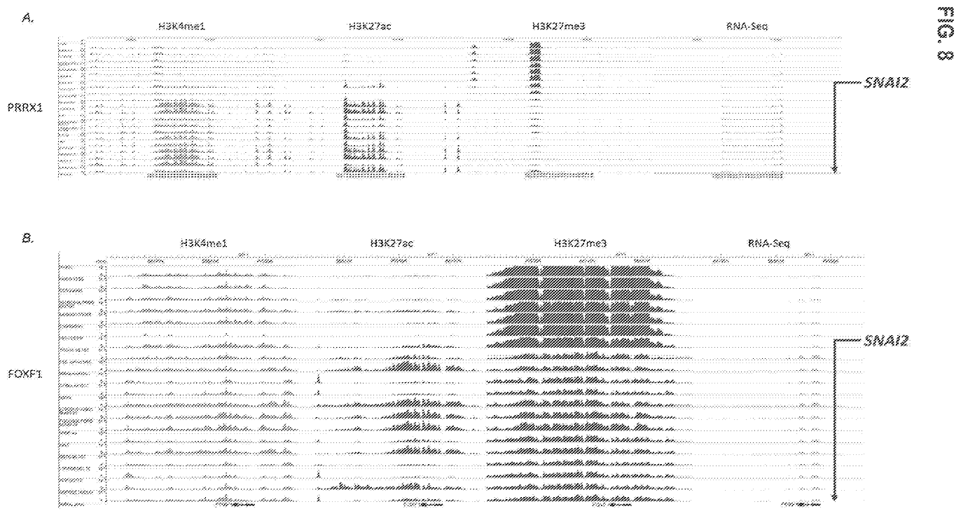

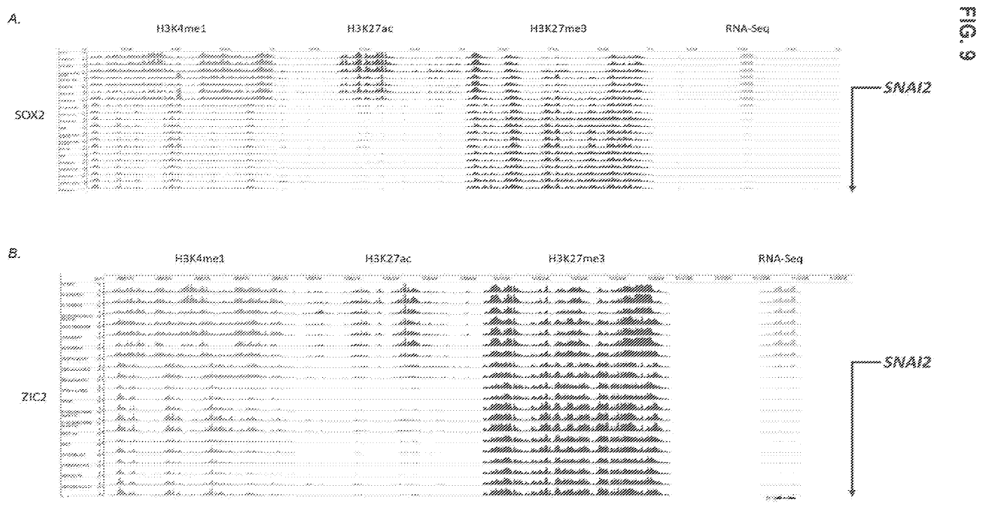

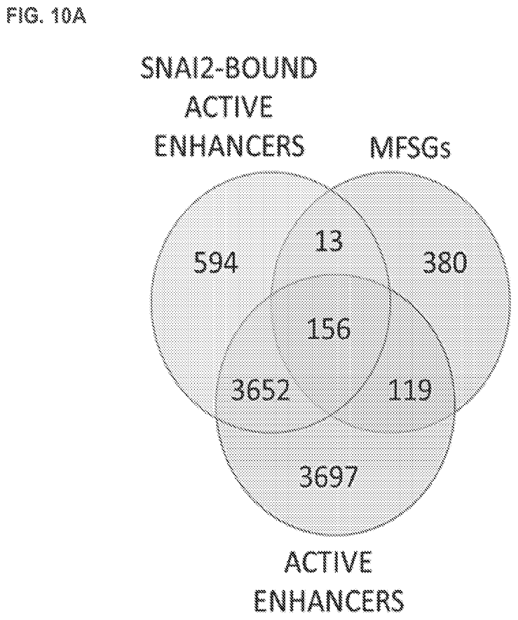

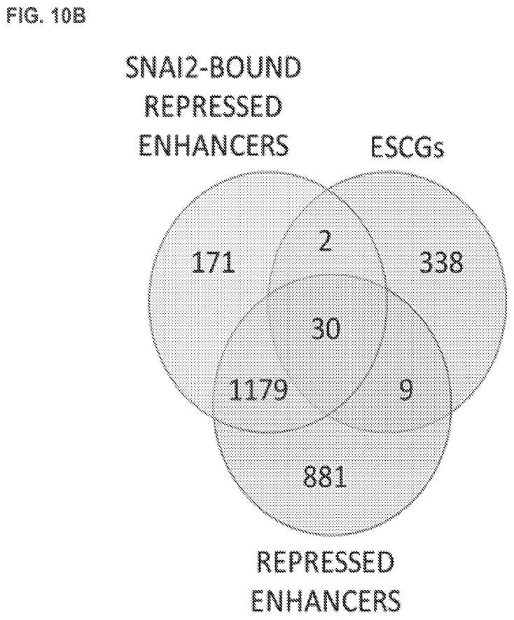

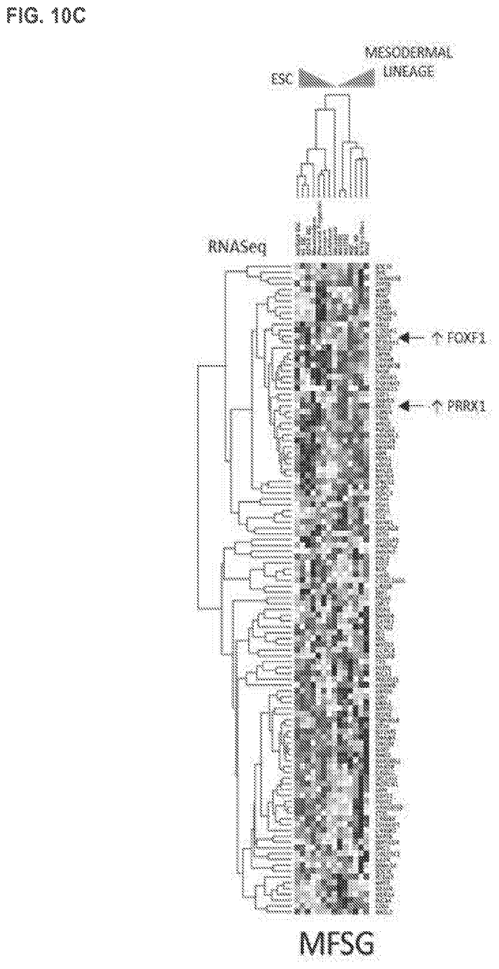

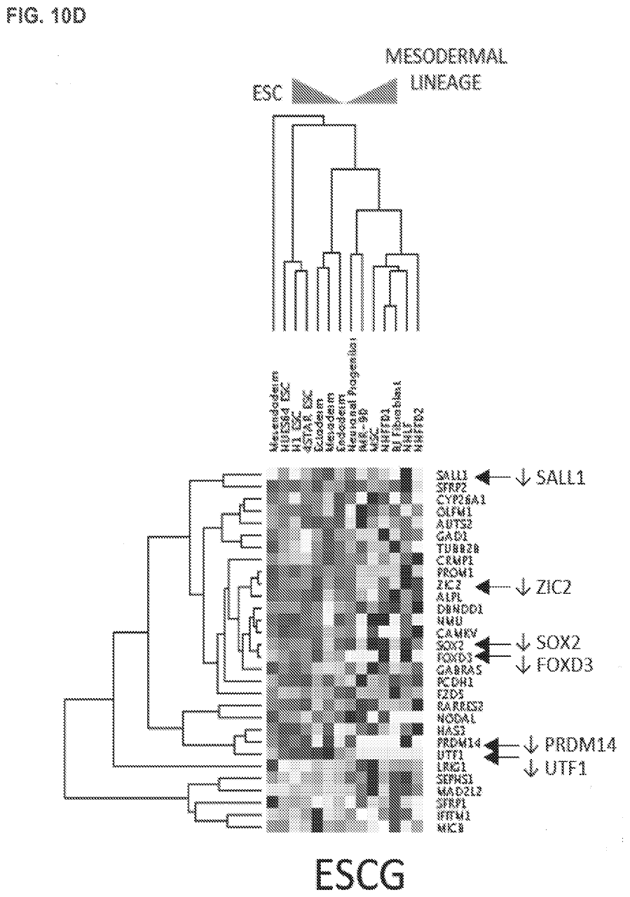

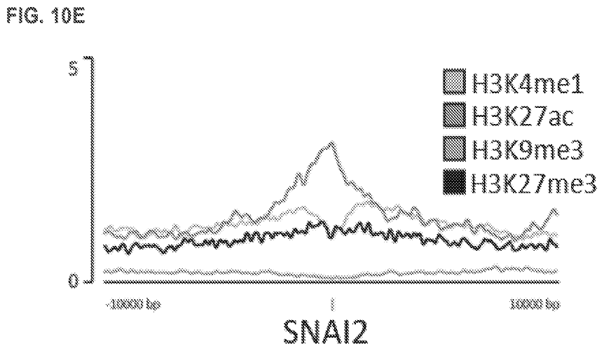

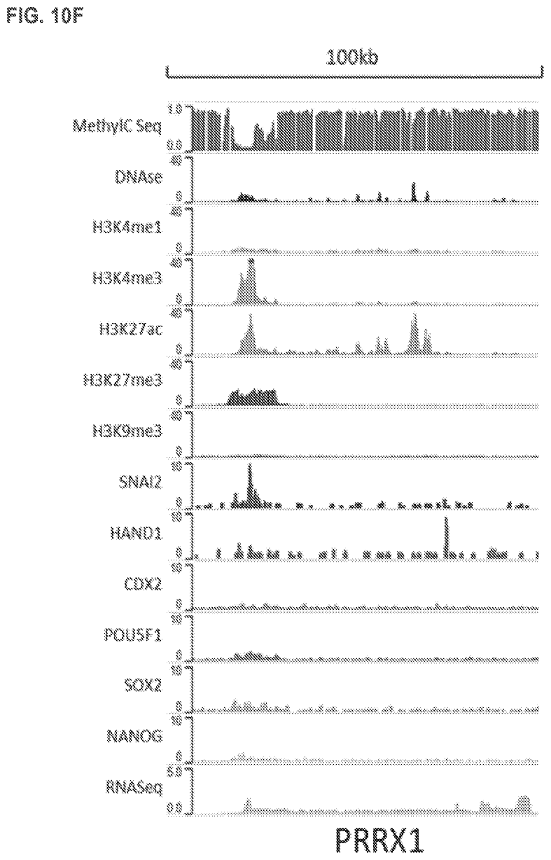

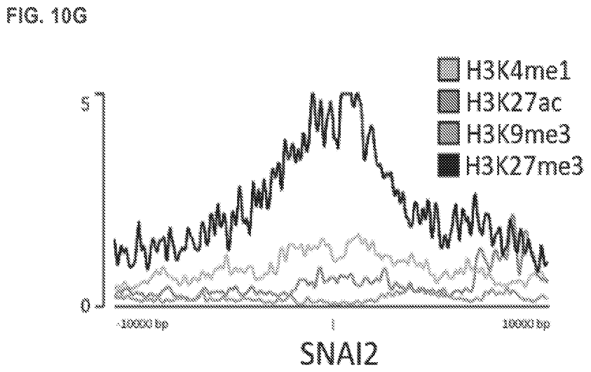

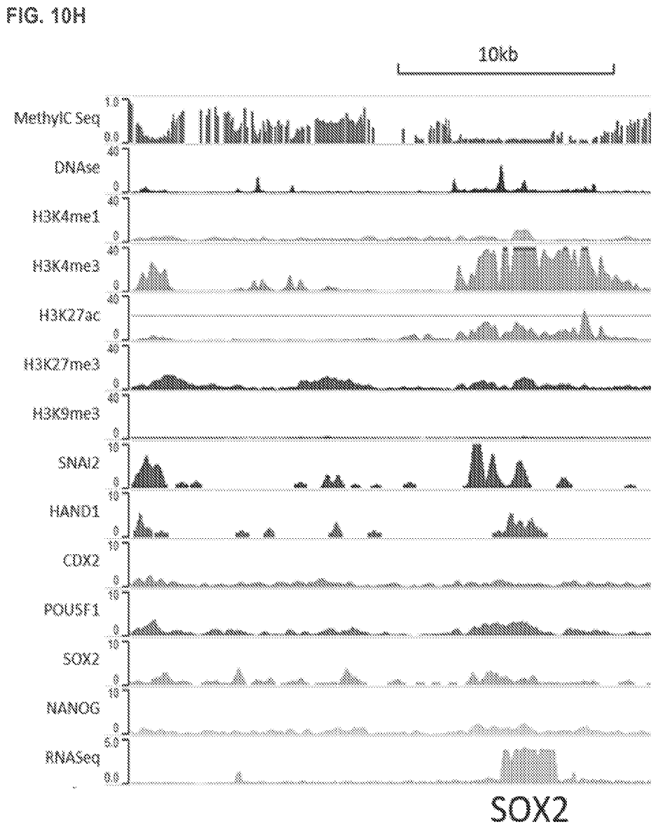

Next, mesodermal TFs (SNAI2, HAND1, CDX2) and ESC TFs (POU5F1, SOX2, NANOG) whose ChIP-Seq data from human HUES64 ESC-derived CD56+mesoderm were available (Tsankov et al., 2015) were analyzed. SNAI2 peaks were found to colocalize with both active enhancers and repressed enhancers more extensively than HAND1 or CDX2 peaks as revealed by TF ChIP-Seq analysis (FIGS. 7A-7H). This suggests that SNAI2 plays a dominant role in the mesoderm in the observed enhanced transcription of key lineage specific genes (see FIGS. 8, 10A-10H; PRRX1, FOXF1) and observed repressed transcription of key ESC genes (see FIGS. 9, 10A-10H; SOX2, ZIC2) as confirmed by matching RNA-Seq analysis. At the SNAI2-bound active MFSG enhancers a marked increase in H3K27 acetylation was observed (FIG. 8) while at the SNAI2-bound repressed ESCG enhancers a marked decrease in H3K27 acetylation accompanied by a synchronous marked increase in H3K27 trimethylation was observed (FIG. 9). RNA-Seq profiling confirmed that the transcriptional effects on SNAI2bound MFSG active enhancers and ESCG repressed enhancers that were observed in human mesoderm were found to both persist and become more prominent and established as mesoderm lineage development proceeds to first fetal and then adult differentiated fibroblasts (FIGS. 8, 9). Because it binds both active enhancers and repressors at the mesoderm stage, SNAI2 acts as a gatekeeper LDTF by regulating both lineage specific gene expression as well as plasticity gene repression.

Further, of the 38 ESC gene targets which have repressors at their gene loci in human mesoderm, the combination of SNAI2, HAND1 and CDX2 bind 33 of them. SNAI2 alone binds 32. Thus, when siSNAI2 is employed to trigger reprogramming of somatic cells, the single biggest impediment (in cells of mesodermal lineage) to ESC gene expression and ability to acquire pluripotency characteristics is effectively removed.

These findings suggest that transient inhibition of a single pivotal gatekeeper TF in a lineage committed cell is adequate for acquisition of cell plasticity and reprogramming.

The dynamic changes in both activating and repressive histone modifications that occur at mesodermal enhancers were observed to be both coincident temporally and overlapping spatially with SNAI2 genomic binding at MFSG and ESCG targets, respectively. SNAI2 itself continues to display strong gene expression along with pervasive persistence of the switch in observed histone modification profiles (at MFSG and ESCG mesodermal enhancers) throughout mesodermal lineage progression across 21 different human cells and tissues (Supplementary FIG. 2,3). SNAI2 thus ostensibly plays a significant if not leading role in both regulating sustained expression of mesodermal lineage genes as well as sustained repression of pluripotency genes, respectively. However, the precise order and sequence of SNAI2 binding events that contribute to regulating the repression of the plasticity program and maintenance of the mesodermal lineage are yet to be established. Similarly, the number and identity of potential co-activator and co-repressor factors that may work in concert with SNAI2 remain unknown. SNAI2 has been reported to recruit Histone Deacetylase 1/2 (HDAC 1/2), decrease H3K27 acetylation activation mark and thereby act as a transcriptional repressor(Soleimani et al., 2012). SNAI2 is also known to work in cooperation with Polycomb Repressive Complex 2 (PRC2), increase H3K27 trimethylation repressive mark and act as a transcriptional repressor(Tien et al., 2015). While the above epigenetic regulatory mechanisms employed by SNAI2 have been demonstrated in other cellular contexts, it remains to be discovered whether similar mechanisms are also exploited by SNAI2 in exerting mesoderm lineage Gatekeeper functions as reported herein.

In conclusion, these results expand our molecular understanding of lineage commitment, specification and fate preservation and reveal that certain pivotal gatekeeper TFs that are master regulators of cell fate can also be considered to constitute a molecular Achilles' heel in a specific cell's identity. They act as the molecular lynchpin of cell fate preservation and may possibly be the single most important reason why cell identity is not immutable. If gatekeeper TFs of a specific cell type can be correctly identified, the siRNA mediated cell-specific transient repression of such gatekeeper TFs can be a tenable strategy to help achieve cell identity switch of such cells to alternate transdifferentiated cell states or to dedifferentiated multipotent/pluripotent cell states, without the need for permanent genetic modification of the targeted cells. Additionally, this may potentially enhance reprogramming efficiencies of exogenous TF/small molecule driven protocols both in terms of cell yield and process time and eventually help realize the goal of reliably obtaining clinically relevant somatic cells or multipotent/pluripotent stem cells for therapeutic use.

### REFERENCES

The references, patents and published patent applications listed below, and all references cited in the specification above are hereby incorporated by reference in their entirety, as if fully set forth herein.
Acharya P S, Majumdar S, Jacob M, Hayden J, Mrass P, Weninger W, Assoian R K, Puré E. Fibroblast migration is mediated by CD44-dependent TGF beta activation. J Cell Sci. 2008 May 1; 121(Pt 9):1393-402.
Acloque H, Adams M S, Fishwick K, Bronner-Fraser M, and Nieto M A, Epithelialmesenchymal transitions: the importance of changing cell state in development and disease. J Clin Invest. 2009 June; 119(6):1438-1449.
Ben-Porath, I., Thomson, M. W., Carey, V. J., Ge, R., Bell, G. W., Regev, A., and Weinberg, R. A. (2008). An embryonic stem cell-like gene expression signature in poorly differentiated aggressive human tumors. Nat Genet 40, 499-507.
Botchan M, Topp W, and Sambrook J. The arrangement of simian virus 40 sequences in the DNA of transformed cells. Cell. 1976 October; 9(2):269-287.
Bulla G A, Aylmer C M, Dust A L, Kurkewich J L, Mire L K, Estanda A B. Genome-wide analysis of hepatic gene silencing in hepatoma cell variants. Genomics. 2012 September; 100(3):176-83.
Bulla G A. and Fournier R E. Genetic analysis of a transcriptional activation pathway by using hepatoma cell variants. Mol Cell Biol. 1994 November; 14(11):7086-7094.
Bulla G A, and Kraus D M. Dissociation of the Hepatic Phenotype from HNF4 and HNF1α Expression. Biosci Rep. 2004 December; 24(6):595-608.
Bulla G A, Luong Q, Shrestha S, Reeb S, and Hickman S. Genome-wide analysis of hepatic gene silencing in mammalian cell hybrids. Genomics. 2010 December; 96(6):323-332.
Barski G., Sorieul S, Cornefert F. 1960. Production dans des cultures in vitro dedeux souches cellulaires en association, decellules de caractere “hybride.” C.R. Acad. Sci. 251:1825-1827.
Cacchiarelli D, Trapnell C, Ziller M J, Soumillon M, Cesana M, Karnik R, Donaghey J, Smith Z D, Ratanasirintrawoot S, Zhang X, Ho Sui S J, Wu Z, Akopian V, Gifford C A, Doench J, Rinn J L, Daley G Q, Meissner A, Lander E S, Mikkelsen T S. Integrative Analyses of Human Reprogramming Reveal Dynamic Nature of Induced Pluripotency. Cell. 2015 Jul. 16; 162(2):412-424.
Cao, N., Huang, Y., Zheng, J., Spencer, C. I., Zhang, Y., Fu, J. D., Nie, B., Xie, M., Zhang, M., Wang, H., et al. (2016). Conversion of human fibroblasts into functional cardiomyocytes by small molecules. Science 352, 1216-1220.
Carey M F, Peterson C L, and Smale S T, Chromatin Immunoprecipitation (ChIP), Cold Spring Harb Protoc. 2009 September; 4(9):1-9.
Consortium, E. P. (2012). An integrated encyclopedia of DNA elements in the human genome. Nature 489, 57-74.
Creyghton, M. P., Cheng, A. W., Welstead, G. G., Kooistra, T., Carey, B. W., Steine, E. J., Hanna, J., Lodato, M. A., Frampton, G. M., Sharp, P. A., et al. (2010). Histone H3K27ac separates active from poised enhancers and predicts developmental state. Proc Natl Acad Sci USA 107, 21931-21936.
Davis, C. A., Hitz, B. C., Sloan, C. A., Chan, E. T., Davidson, J. M., Gabdank, I., Hilton, J. A., Jain, K., Baymuradov, U.K., Narayanan, A. K., et al. (2018). The Encyclopedia of DNA elements (ENCODE): data portal update. Nucleic Acids Res 46, D794-D801.
Davis R L, Weintraub H, Lassar A B. Expression of a single transfected cDNA converts fibroblasts to myoblasts. Cell. 1987 Dec. 24; 51(6):987-1000.
Ebrahimi B. Reprogramming barriers and enhancers: strategies to enhance the efficiency and kinetics of induced pluripotency. Cell Regen (Lond). 2015 Nov. 11; 4:10.
Evseenko, D., Zhu, Y., Schenke-Layland, K., Kuo, J., Latour, B., Ge, S., Scholes, J., Dravid, G., Li, X., MacLellan, W. R., et al. (2010). Mapping the first stages of mesoderm commitment during differentiation of human embryonic stem cells. Proc Natl Acad Sci USA 107, 13742-13747.
Furey T S. ChIP-seq and beyond: new and improved methodologies to detect and characterize protein-DNA interactions, Nat Rev Genet. 2012 December; 13(12):840-852.
Gingold J A, Fidalgo M, Guallar D, Lau Z, Sun Z, Zhou H, Faiola F, Huang X, Lee D F, Waghray A, Schaniel C, Felsenfeld D P, Lemischka I R, Wang J. A genome-wide RNAi screen identifies opposing functions of Snai1 and SNAI2 on the Nanog dependency in reprogramming. Mol Cell. 2014 Oct. 2; 56(1):140-52.
Gokbuget, D., and Blelloch, R. (2019). Epigenetic control of transcriptional regulation in pluripotency and early differentiation. Development 146.
Gomes I C, Acquarone M, Maciel Rde M, Erlich R B, Rehen S K. Analysis of pluripotent stem cells by using cryosections of embryoid bodies. J Vis Exp. 2010 Dec. 8; (46). pii: 2344.
Graf T. Historical origins of transdifferentiation and reprogramming. Cell Stem Cell. 2011 Dec. 2; 9(6):504-16.
Hikichi T, Matoba R, Ikeda T, Watanabe A, Yamamoto T, Yoshitake S, Tamura-Nakano M, Kimura T, Kamon M, Shimura M, Kawakami K, Okuda A, Okochi H, Inoue T, Suzuki A, Masui S. Transcription factors interfering with dedifferentiation induce cell type-specific transcriptional profiles. Proc Natl Acad Sci USA. 2013 Apr. 16; 110(16):6412-7.
Hou, P., Li, Y., Zhang, X., Liu, C., Guan, J., Li, H., Zhao, T., Ye, J., Yang, W., Liu, K., et al. (2013). Pluripotent stem cells induced from mouse somatic cells by small-molecule compounds. Science 341, 651-654.
Jackson M, Taylor A H, Jones E A, Forrester L M. The culture of mouse embryonic stem cells and formation of embryoid bodies. Methods Mol Biol. 2010; 633:1-18.
Kao K N, Constabel F, M. R. Michayluk, and O. L. Gamborg. Plant Protoplast Fusion and Growth of Intergeneric Hybrid Cells. Planta. 1974 January; 120(3):215-227.
Killary A M, and Fournier REK. A genetic analysis of extinction: trans-dominant loci regulate expression of liver-specific traits in hepatoma hybrid cells. Cell. 1984 September; 38(2):523-534.
Killary A M, Lugo T G, and Fournier REK. Isolation of Thymidine Kinase-Deficient Rat Hepatoma Cells by Selection with Bromodeoxyuridine, Hoechst 33258, and Visible Light. Biochem Genet. 1984 April; 22(3-4):201-213.
Killary A M, and Fournier REK. A genetic analysis of extinction: trans-dominant loci regulate expression of liver-specific traits in hepatoma hybrid cells. Cell. 1984 September; 38(2):523-534.
Kogut I, McCarthy S M, Pavlova M, Astling D P, Chen X, Jakimenko A, Jones K L, Getahun A, Cambier J C, Pasmooij A M G, Jonkman M F, Roop D R, Bilousova G. High-efficiency RNA-based reprogramming of human primary fibroblasts. Nat Commun. 2018 Feb. 21; 9(1):745.
Liu K, Yu C, Xie M, Li K, Ding S. Chemical Modulation of Cell Fate in Stem Cell Therapeutics and Regenerative Medicine. Cell Chem Biol. 2016 Aug. 18; 23(8):893-916.
Mikkelsen T S, Hanna J, Zhang X, Ku M, Wernig M, Schorderet P, Bernstein B E, Jaenisch R, Lander E S, Meissner A. Dissecting direct reprogramming through integrative genomic analysis. Nature. 2008 Jul. 3; 454(7200):49-55.
Nashun B, Hill P W, Hajkova P. Reprogramming of cell fate: epigenetic memory and the erasure of memories past. EMBO J. 2015 May 12; 34(10):1296-308.
Ozsolak F and Milos M P. RNA sequencing: advances, challenges and opportunities. Nat Rev Genet. 2011 February; 12(2):87-98.
Polo J M, Anderssen E, Walsh R M, Schwarz B A, Nefzger C M, Lim S M, Borkent M, Apostolou E, Alaei S, Cloutier J, Bar-Nur O, Cheloufi S, Stadtfeld M, Figueroa M E, Robinton D, Natesan S, Melnick A, Zhu J, Ramaswamy S, Hochedlinger K. A molecular roadmap of reprogramming somatic cells into iPS cells. Cell. 2012 Dec. 21; 151(7):1617-32.
Ringertz, N R, and Savage R E. The Selection of Heterokaryons and Cell Hybrids Using the Biochemical Inhibitors Iodoacetamide and Diethylpyrocarbonate. 1976. Cell hybrids.
Roadmap Epigenomics, C., Kundaje, A., Meuleman, W., Ernst, J., Bilenky, M., Yen, A., Heravi-Moussavi, A., Kheradpour, P., Zhang, Z., Wang, J., et al. (2015). Integrative analysis of 111 reference human epigenomes. Nature 518, 317-330.
Shendure J and Aiden E L. The expanding scope of DNA sequencing, Nat Biotechnol. 2012 November; 30(11):1084-1094.
Sivaguru M, Liu J, Kochian L V. Targeted expression of SbMATE in the root distal transition zone is responsible for sorghum aluminum resistance. Plant J. 2013 October; 76(2):297-307
Smith Z D, Sindhu C, Meissner A. Molecular features of cellular reprogramming and development. Nat Rev Mol Cell Biol. 2016 March; 17(3):139-54.
Soleimani, V. D., Yin, H., Jahani-Asl, A., Ming, H., Kockx, C. E., van Ijcken, W. F., Grosveld, F., and Rudnicki, M. A. (2012). Snai1 regulates MyoD binding-site occupancy to direct enhancer switching and differentiation-specific transcription in myogenesis. Mol Cell 47, 457-468.
Sorieul S, Ephrussi B. Karyological demonstration of hybridization of mammalian cells in vitro. Nature. 1961 190:653-654.
Srivastava D, DeWitt N. In vivo Cellular Reprogramming: The Next Generation. Cell. 2016 Sep. 8; 166(6):1386-1396.
Strutz F, Okada H, Lo C W, Danoff T, Carone R L, Tomaszewski J E, and E. G. Neilson. Identification and Characterization of a Fibroblast Marker: FSP1. J Cell Biol. 1995 July; 130(2):393-405.
Takahashi K, Yamanaka S. A decade of transcription factor-mediated reprogramming to pluripotency. Nat Rev Mol Cell Biol. 2016 March; 17(3):183-93.
Takahashi K, Yamanaka S. Induction of pluripotent stem cells from mouse embryonic and adult fibroblast cultures by defined factors. Cell. 2006 Aug. 25; 126(4):663-76.
Thoma, E. C., Merkl, C., Heckel, T., Haab, R., Knoflach, F., Nowaczyk, C., Flint, N., Jagasia, R., Jensen Zoffmann, S., Truong, H. H., et al. (2014). Chemical conversion of human fibroblasts into functional Schwann cells. Stem Cell Reports 3, 539-547.
Tien, C. L., Jones, A., Wang, H., Gerigk, M., Nozell, S., and Chang, C. (2015). Snail2/Slug cooperates with Polycomb repressive complex 2 (PRC2) to regulate neural crest development. Development 142, 722-731.
Tomaru Y, Hasegawa R, Suzuki T, Sato T, Kubosaki A, Suzuki M, Kawaji H, Forrest A R, Hayashizaki Y; FANTOM Consortium, Shin J W, Suzuki H. A transient disruption of fibroblastic transcriptional regulatory network facilitates trans-differentiation. Nucleic Acids Res. 2014 August; 42(14):8905-13
Tosic, J., Kim, G. J., Pavlovic, M., Schroder, C. M., Mersiowsky, S. L., Barg, M., Hofherr, A., Probst, S., Kottgen, M., Hein, L., et al. (2019). Eomes and Brachyury control pluripotency exit and germ-layer segregation by changing the chromatin state. Nat Cell Biol 21, 1518-1531.
Tsankov, A. M., Gu, H., Akopian, V., Ziller, M. J., Donaghey, J., Amit, I., Gnirke, A., and Meissner, A. (2015). Transcription factor binding dynamics during human ES cell differentiation. Nature 518, 344-349.
Vierbuchen T, Ostermeier A, Pang Z P, Kokubu Y, Südhof T C, Wernig M. Direct conversion of fibroblasts to functional neurons by defined factors. Nature. 2010, Feb. 25; 463(7284):1035-41.
Weltner J, Balboa D, Katayama S, Bespalov M, Krjutškov K, Jouhilahti E M, Trokovic R, Kere J, Otonkoski T. Human pluripotent reprogramming with CRISPR activators. Nat Commun. 2018 Jul. 6; 9(1):2643.
Willis B C, Liebler J M, Luby-Phelps K, Nicholson A G, Crandall E D, Du Bois R M, Borok Z. Induction of epithelial-mesenchymal transition in alveolar epithelial cells by transforming growth factor-beta1: potential role in idiopathic pulmonary fibrosis, Am J Pathol. 2005 May; 166(5):1321-1332.
Xie X, Fu Y, Liu J. Chemical reprogramming and transdifferentiation. Curr Opin Genet Dev. 2017 October; 46:104-113.
Xu J, Du Y, Deng H. Direct lineage reprogramming: strategies, mechanisms, and applications. Cell Stem Cell. 2015 Feb. 5; 16(2):119-34.
Yang C S, Lopez C G, Rana T M. Discovery of nonsteroidal anti-inflammatory drug and anticancer drug enhancing reprogramming and induced pluripotent stem cell generation. Stem Cells. 2011 October; 29(10):1528-36.
Zentner, G. E., Tesar, P. J., and Scacheri, P. C. (2011). Epigenetic signatures distinguish multiple classes of enhancers with distinct cellular functions. Genome Res 21, 1273-1283.
Zhao X, Ruan Y, Wei C L. Tackling the epigenome in the pluripotent stem cells, J Genet Genomics. 2008 July; 3 5 (7):403-412.

I/We claim:

1. A somatic fibroblast cell comprising an siRNA or shRNA molecule that inhibits expression of a single lineage defining transcription factor, wherein the somatic fibroblast cell is capable of transdifferentiating to a somatic cell of alternate lineage in the absence of viral or non-viral delivery of exogenous transcription factors and/or modulators.

2. The somatic fibroblast cell of claim 1, wherein the single lineage defining transcription factor is SNAI2, PRRX1, HAND1, CDX2, or a combination thereof.

3. The somatic fibroblast cell of any one of claims 1-2, wherein the somatic cell of alternate lineage is a somatic cell of adipocytic lineage, a somatic cell of osteogenic lineage, or a somatic cell of chondrogenic lineage.

4. A somatic fibroblast cell comprising an siRNA or shRNA molecule that inhibits expression of a single lineage defining transcription factor, wherein the somatic fibroblast cell is capable of dedifferentiating to a multipotent or pluripotent stem cell in the absence of viral or non-viral delivery of exogenous transcription factors and/or modulators.

5. The somatic fibroblast cell of claim 4, wherein the single lineage defining transcription factor is SNAI2, PRRX1, HAND1, CDX2, or a combination thereof.

6. The somatic fibroblast cell of any one of claims 4-5, wherein the multipotent or pluripotent stem cell is a dedifferentiated multipotent mesenchymal stem cell or a dedifferentiated pluripotent stem cell.

7. A population of transdifferentiated somatic cells comprising a population of reprogrammed fibroblast cells lacking permanent genetic modification, wherein the population of reprogrammed fibroblast cells were converted to the population of transdifferentiated somatic cells from a population of lineage committed fibroblast cells at an efficiency of more than 1%.

8. The population of transdifferentiated somatic cells of claim 7, wherein the population of transdifferentiated somatic cells is a population of somatic cells of adipocytic lineage, a population of somatic cells of osteogenic lineage, or a population of somatic cells of chondrogenic lineage.

9. The population of transdifferentiated somatic cells of claim 7 or 8 for use in a tissue reconstruction procedure.

10. The population of transdifferentiated somatic cells of any one of claims 7-9 wherein the tissue reconstruction procedure is a reconstructive plastic surgery procedure or a reconstructive orthopedic surgery procedure.

11. A population of dedifferentiated stem cells comprising a population of reprogrammed fibroblast cells lacking permanent genetic modification, wherein the population of reprogrammed fibroblast cells were induced to become the population of dedifferentiated stem cells from a population of lineage committed fibroblast cells at an efficiency of more than 1%.

12. The population of dedifferentiated stem cells of claim 11, wherein the population of dedifferentiated stem cells is a population of dedifferentiated multipotent mesenchymal stem cells or a population of dedifferentiated pluripotent stem cells.

13. The population of dedifferentiated stem cells of claim 11 or 12 for use in a tissue reconstruction procedure.

14. The population of dedifferentiated stem cells of claim 11 or 12 for use in a wound healing application.

15. The population of dedifferentiated stem cells of claim 11 or 12 for use in a transplantation procedure.

16. A cell culture system for producing a population of transdifferentiated somatic cells comprising:
a population of lineage committed somatic cells;
an siRNA or shRNA molecule that inhibits expression of a single lineage defining transcription factor; and
a culture medium comprising a cell culture media specific to the population of transdifferentiated somatic cells;
wherein the cell culture system does not include an exogenous transcription factor or modulator specific to the transdifferentiated somatic cells.

17. The cell culture system of claim 16, wherein the population of lineage committed somatic cells are lineage committed fibroblast cells.

18. The cell culture system of claim 16 or 17, wherein the single lineage defining transcription factor is SNAI2, PRRX1, NAND1, CDX2, or a combination thereof.

19. The cell culture system of any one of claims 16-18, wherein the population of transdifferentiated somatic cells are of adipocytic lineage, osteogenic lineage, or chondrogenic lineage.

20. A cell culture system for producing a population of dedifferentiated stem cells comprising:
a population of lineage committed somatic cells;
an siRNA or shRNA molecule that inhibits expression of a single lineage defining transcription factor; and
a culture medium comprising a stem cell media specific to the population of dedifferentiated stem cells;
wherein the cell culture system does not include an exogenous transcription factor or modulator specific to the transdifferentiated somatic cells.

21. The cell culture system of claim 20, wherein the population of lineage committed somatic cells are lineage committed fibroblast cells.

22. The cell culture system of claim 20 or 21, wherein the single lineage defining transcription factor is SNAI2, PRRX1, HAND1, CDX2, or a combination thereof.

23. The cell culture system of any one of claims 20-22, wherein the population of dedifferentiated stem cells is a population of dedifferentiated multipotent mesenchymal stem cells or a population of dedifferentiated pluripotent stem cells.

24. A method for producing a transdifferentiated somatic cell comprising:
introducing an siRNA or shRNA molecule that transiently inhibits expression of a single lineage defining transcription factor to an lineage committed somatic cell; and
incubating the lineage committed somatic cell in a culture media specific to the transdifferentiated somatic cell;
wherein the method for producing the transdifferentiated somatic cell is performed in the absence of viral or non-viral delivery of exogenous transcription factors and/or modulators.

25. The method of claim 24, wherein the lineage committed somatic cell is an lineage committed fibroblast cell.

26. The method of claim 24 or 25, wherein the single lineage defining transcription factor is SNAI2, PRRX1, HAND1, CDX2, or a combination thereof.

27. The method of any one of claims 24-26, wherein the transdifferentiated somatic cell is a cell of adipocytic lineage, a cell of osteogenic lineage, or a cell of chondrogenic lineage.

28. A method for producing a dedifferentiated stem cell comprising:
introducing an siRNA or shRNA molecule that transiently inhibits expression of a single lineage defining transcription factor to an lineage committed somatic cell; and
incubating the lineage committed somatic cell in a culture media specific to the dedifferentiated stem cell;
wherein the method for producing the dedifferentiated stem cell is performed in the absence of viral or non-viral delivery of exogenous transcription factors and/or modulators.

29. The method of claim 28, wherein the lineage committed somatic cell is an lineage committed fibroblast cell.

30. The method of claim 28 or 29, wherein the single lineage defining transcription factor is SNAI2, PRRX1, NAND1, CDX2, or a combination thereof.

31. The method of any one of claims 28-30, wherein the dedifferentiated stem cell is an dedifferentiated multipotent mesenchymal stem cell or an dedifferentiated pluripotent stem cell.

32. An autologous tissue graft comprising a population of transdifferentiated somatic cells, wherein:
(i) the population of transdifferentiated somatic cells is derived from a population of lineage committed somatic cells obtained from a subject for use in a tissue reconstruction procedure, and
(ii) the population of transdifferentiated somatic cells comprises a population of reprogrammed fibroblast cells lacking permanent genetic modification, wherein the population of reprogrammed fibroblast cells were converted to the population of transdifferentiated somatic cells from a population of lineage committed fibroblast cells at an efficiency of more than 1%.

33. The autologous tissue graft of claim 32, wherein the population of transdifferentiated somatic cells is produced using the method of any one of claims 30-35.

34. An autologous tissue graft comprising a population of dedifferentiated stem cells, wherein
(i) the population of dedifferentiated stem cells is derived from a population of lineage committed somatic cells obtained from a subject for use in a tissue reconstruction procedure, a wound healing application, or a transplantation procedure, and
(ii) the population of dedifferentiated stem cells comprising a population of reprogrammed fibroblast cells lacking permanent genetic modification, wherein the population of reprogrammed fibroblast cells were induced to become the population of dedifferentiated stem cells from a population of lineage committed fibroblast cells at an efficiency of more than 1%.

35. The autologous tissue graft of claim 34, wherein the population of dedifferentiated stem cells is produced using the method of any one of claims 28-31.

36. A method for treating a condition in a subject comprising grafting or transplanting a population of reprogrammed somatic cells, dedifferentiated stem cells, or transdifferentiated somatic cells into or onto a tissue or organ of the subject.

37. The method of claim 36, wherein the a population of reprogrammed somatic cells, dedifferentiated stem cells, or transdifferentiated somatic cells are part of an autologous tissue graft.

38. The somatic fibroblast cell, cell culture system, or method of any one of claims 1-6, 16-31, wherein the siRNA or shRNA molecule is an siRNA molecule comprising an siRNA sequence and a passenger strand sequence of Table 1 or Table 2.

39. The somatic fibroblast cell, cell culture system, or method of any one of claims 1-6, 16-31, wherein the siRNA or shRNA molecule is an shRNA molecule comprising an siRNA sequence, a passenger strand sequence, and a loop sequence of Table 1 or Table 2.
